(19) United States
(12) Patent Application Publication
(10) Pub. No.: US 2004/0005294 A1
(43) Pub. Date: Jan. 8, 2004

(54) IGFBP-3 in the diagnosis and treatment of cancer

(76) Inventor: Ho-Young Lee, Houston, TX (US)

Correspondence Address: Steven L. Highlander, Fulbright & Jaworski L.L.P., Suite 2400, 600 Congress Avenue, Austin, TX, 78701, US

(21) Appl. No.: 10/377,142

(22) Filed: Feb. 25, 2003

Related U.S. Application Data
(60) Provisional application No. 60/359,536, filed on Feb. 25, 2002.

(51) Int. Cl. A61K 48/00; A61K 38/18

(52) U.S. Cl. 424/93.2; 514/2; 514/44

(57) ABSTRACT

The present invention provide for methods of inhibiting cancer cell growth using IGFBP-3 polypeptides and expression constructs coding therefor. In a particular aspect, the invention provides adenoviral constructs expressing IGFBP-3, and their use to inhibit non-small cell lung cancer. In addition, IGFBP-3 expression can be diagnostic of cancer development and progression. Methods for assessing IGFBP-3 expression, for example using promoter methylation assays, are described.

The present invention claims priority to U.S. Provisional Patent Application Serial No. 60/359,536 filed on Feb. 25, 2002. The entire text of the above-referenced disclosure is specifically incorporated herein by reference without disclaimer.

The government may owns rights in the present invention pursuant to grant number RP33763 from the National Institutes of Health.

### BACKGROUND OF THE INVENTION

1. Field of the Invention

The present invention relates generally to the fields of oncology and molecular biology. More particularly, it concerns insulin growth factor binding protein 3 (IGFBP 3) in treating lung cancer.

2. Description of Related Art

Non-small cell lung cancer (NSCLC) accounts for about 75-80% of lung cancer cases and carries a 5-year survival rate of about 10-15% for all stages (Mitsudomi et al., 1992). Surgical resection is the treatment of choice for patients with stage I or II cancer, whereas patients with later stages of disease are treated with combinations of surgery, chemotherapy, and radiation therapy, all of which have significant side effects. Despite these treatments, the survival rate of patients with NSCLC remains low (Nemunatis et al., 2000), and new treatment strategies are urgently needed.

### SUMMARY OF THE INVENTION

Thus, in accordance with the present invention, there is provided a method for inhibiting the growth of a lung cancer cell comprising contacting the cell with an IGFBP-3. The lung cancer cell may be a non-small cell lung cancer cell. The method may further comprise contacting said lung cancer cell with a chemotherapeutic, radiotherapy or non-IGFBP-3 gene therapy. The IGFBP-3 may be provided as a protein, i.e., delivered in a pharmaceutical formulation, or it may be provided by virtue of a vector comprising an IGFBP-3-encoding nucleic acid under the control of a promoter active in said hyperproliferative cell. The vector may be a non-viral vector, for example, where the non-viral vector is encapsulated in a lipid. The vector may also be a viral vector, for example, an adenoviral vector, an adeno-associated viral vector, a retroviral vector, a herpesviral vector, a vaccinia viral vector and a papillomavirus vector. In a particular embodiment, the viral vector may be an adenoviral vector, for example, where the adenoviral vector is replication-deficient. The adenoviral vector may lack at least a portion of the E1 region, and the the nucleic acid encoding IGFBP-3 may be inserted in the E1 region. The said nucleic acid may also comprise a polyadenylation signal. The promoter may be an inducible promoter, a tissue-specific promoter (e.g., a cancer tissue-specific promoter) or a constitutive promoter (e.g., CMV IE).

In another embodiment, there is provided a method for treating cancer in a subject comprising administering IGFBP-3 to said subject. The cancer may be lung cancer, breast cancer, pancreatic cancer, liver cancer, stomach cancer, colon cancer, ovarian cancer, uterine cancer, prostate cancer, testicular cancer, head & neck cancer, skin cancer, brain cancer, esophageal cancer or blood cancer. The lung cancer may be non-small cell lung cancer. The IGFBP-3 may be administered to said subject as a protein in a pharmaceutical formulation, or produced from a vector introduced into a cell of said subject, said vector comprising an IGFBP-3-encoding nucleic acid under the control of a promoter active in said hyperproliferative cell. The cell of said subject may be a cancer cell. The IGFBP-3 or IGFBP-3 vector may be administered intratumorally, administered into tumor vasculature, administered regional to a tumor, administered to airway epithelia by aerosol or administered systemically.

The method may further comprise administering to said subject a second cancer therapy, such as protein therapy, gene therapy, radiation therapy, chemotherapy or surgery. The protein therapy may be selected from antibody therapy, cytokine therapy, pro-apoptotic protein therapy, peptide hormone therapy and toxin therapy. The gene therapy may be selected from tumor suppressor therapy, antisense oncogene therapy, pro-apoptotic gene therapy, anti-oncogene single-chain antibody gene therapy, cytokine gene therapy, peptide hormone gene therapy and toxin gene therapy. A particular gene therapy is one based on deguelin. The radiation therapy may be selected from gamma irradiation, x-irradiation, ultraviolet irradiation, and microwave irradiation. The chemotherapy may be selected from cisplatin, carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate. The second cancer therapy may be surgery. The method may further comprise administering to said subject a third cancer therapy. The second cancer therapy may be provided before IGFBP-3 therapy, after IGFBP-3 therapy or at the same time as IGFBP-3 therapy.

In yet another embodiment, there is provided a method for predicting or diagnosing cancer in a subject comprising assessing the expression of IGFBP-3 in a cell of said subject, wherein a reduced expression of IGFBP-3, as compared to that seen in a normal cell, is indicate of a risk or presence of cancer. The cell may be a tumor cell or a non-tumor cell. Assessing the expression of IGFBP-3 may comprise measuring IGFBP-3 protein levels in said cell, measuring IGFBP-3 transcript levels in said cell, or determining the methylation state of the IGFBP-3 promoter, such as by methylation specific PCR. Assessing the expression of IGFBP-3 may also comprise determining the presence of a mutation in the IGFBP-3 coding region, for example, by RFLP analysis, sequencing, and DNAse protection. The method may further comprise assessing IGFBP-3 expression in a cell from a healthy patient, or assessing IGFBP-3 expression in a non-tumor cell from said subject.

In still yet another embodiment, there is provided a method for predicting the efficacy of a cancer therapy on a subject comprising assessing the expression of IGFBP-3 in a cell of said subject. Assessing the expression of IGFBP-3 may comprise determining the methylation state of the IGFBP-3 promoter, such as by using methylation specific PCR™.

In still a further embodiment, there is provided a method for predicting the survival of a subject having cancer comprising assessing the expression of IGFBP-3 in a cell of said subject. Assessing may comprise determining the methylation state of the IGFBP-3 promoter, for example, using methylation specific PCR™.

In still an even further embodiment, there is provided a method for predicting the recurrence of a cancer in a subject comprising assessing the expression of IGFBP-3 in a cell of said subject. Assessing may comprise determining the methylation state of the IGFBP-3 promoter, for example, using methylation specific PCR™.

In yet an even further embodiment, there is provided a method for predicting metastasic cancer in a subject comprising assessing the expression of IGFBP-3 in a cell of said subject. Assessing may comprise determining the methylation state of the IGFBP-3 promoter, for example, using methylation specific PCR™.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1C—The Kaplan-Meier curves of patients with stage I NSCLC stratified by IGFBP-3 expression. The probability of 5-year overall survival of patients whose tumors showed loss of IGFBP-3 expression was 51.3% as compared with 71.3% for patients whose tumors showed IGFBP-3 expression (FIG. 1A). The probability of 5-year disease-specific survival of patients whose tumors showed loss of IGFBP-3 expression was 64.3% as compared with 85.7% in patients whose tumors showed IGFBP-3 expression (FIG. 1B). The 5-year disease-free survival rate for patients whose tumors showed loss of IGFBP-3 expression was 54.4% as compared with 71.4% for patients whose tumors showed IGFBP-3 expression (FIG. 1C).

FIGS. 2A-2C—Ad5CMV-BP3 infection increases IGFBP-3 expression in NSCLC cell lines. (FIG. 2A) Whole-cell lysates isolated from the indicated NSCLC cell lines were subjected to Western blot analysis for IGFBP-3 expression. (FIG. 2B) H1299 NSCLC cells were untreated (Con) or infected with the indicated titers (particles/cell) of Ad5CMV-BP3 or the parental vector Ad5CMV for 3 days. IGFBP-3 in the cell (C) or secreted into the medium (S) was detected by Western blot analysis. The time course of IGFBP-3 expression was determined in H1299 cells that were untreated (Con) or infected with 1×10⁴ particles/cell of Ad5CMV-BP3 or Ad5CMV. β-actin was used as a loading control. (FIG. 2C) Western ligand blot analysis was performed on conditioned media from cells infected with the indicated dose of Ad5CMV-BP3 or Ad5CMV for 3 days, using [¹²⁵I ] IGF-I as a probe. Recombinant IGFBP-3 (20 ng) was used as a positive control. The positions of specific forms of IGFBP-3 are indicated.

FIGS. 3A-3B—Ad5CMV-BP3 inhibits the growth of NSCLC cells in an IGF-dependent way. (FIG. 3A) (Left) The effects of IGF-I on the growth of indicated NSCLC cell lines were measured by MTT assays of cells incubated in serum-free medium with or without indicated doses of IGF-I for 3 days. Results are expressed relative to the density of cells incubated in serum-free medium. (Right) Same cell lines were infected with the indicated doses (particles/cell) of Ad5CMV-BP3 or Ad5CMV and incubated in a serum-free medium with or without 100 ng/ml or 250 ng/ml IGF-I. Results are expressed relative to the density of untreated cells incubated in a same medium that is either serum-free or containing 100 ng/ml or 250 ng/ml IGF-I, respectively. (FIG. 3B) The effects of IGF-I on the growth of NHBE cells were measured by MTT assays of cells infected with the indicated doses (particles/cell) of Ad5CMV-BP3 or Ad5CMV and incubated in a serum-free medium with or without 250 ng/ml IGF-I. MTT assays on infected cells were performed after 3 days of incubation. Results are expressed relative to the density of cells incubated in serum-free medium. Each value is the mean (±SD) from 6 identical wells.

FIG. 4—Ad5CMV-BP3 inhibits anchorage-independent growth of NSCLC cells. H1299 cells were infected with Ad5CMV-BP3 and plated in RPMI 1640 medium containing 0.2% agarose on top of a base of 0.5% agarose in the culture medium. After 1 week, cells were infected again with the same dose of adenovirus. After 2 weeks, colonies >125 μm in diameter were counted under a microscope. Each value represents the mean (±SD) from 3 independent studies.

FIG. 5—Growth of NSCLC xenografts is inhibited by injection of Ad5CMV-BP3. H1299 cells were injected into the dorsal flank of athymic nude mice. Once tumor volume reached approximately 75 mm³, 1×10¹⁰ viral particles of the indicated adenovirus or buffer alone (PBS) as a control was intratumorally injected. Tumors were measured every day, and results were expressed as the mean (±SD) tumor volume (calculated from 5 mice) relative to the tumor volume at the time of adenoviral injection (day 0).

FIGS. 6A-6C—IGFBP-3 induces apoptosis in NSCLC cells. (FIG. 6A) Flow cytometry was performed in H1299 cells infected with Ad5CMV-BP3 or Ad5CMV using APO-BRDU staining. Living gating of the forward and orthogonal scatter channels was used to exclude debris and to selectively acquire cell events. All values reflect the percentage of cells as determined by light scatter. The percentage of dead cells was determined by FACS analysis of PI-stained nuclei. (FIG. 6B) Nucleosomal DNA fragmentation analysis was performed on DNA isolated from untreated (Con), Ad5CMV-infected, and Ad5CMV-BP3-infected H1299 cells. (FIG. 6C) The regulation of Bcl-2, Bax, and caspase-3 proenzyme (32-kDa) and the cleavage of PARP by Ad5CMV-BP3 were examined by Western blot analysis on H1299 cells infected with the indicated doses of adenovirus for 3 days.

FIG. 7—IGFBP-3 expression and apoptosis are induced in NSCLC tumor xenografts following a single injection of Ad5CMV-BP3. Immunocytochemical analysis for IGFBP-3 expression and TUNEL analysis were performed on the tissues from H1299 cell-induced tumor nodules injected with Ad5CMV-BP3 or Ad5CMV. The cells positive upon staining for IGFBP-3 and for apoptosis are identified by red and green fluorescence, respectively.

FIGS. 8A-8B—IGFBP-3 inhibits the PI3K and MAPK pathways in NSCLC cells. (FIG. 8A) The expression of pAkt (Ser473), Akt, pGSK-3β (Ser9), and GSK-3β were measured by Western blot analysis in H1299 cells untreated (Con) or infected with the indicated dose of adenovirus. (FIG. 8B) MAPK activity was determined by an immune complex kinase assay using myelin basic protein as a substrate. Total ERK1/2 expression was examined by Western blot analysis.

FIGS. 9A-9B—(FIG. 9A) IGFBP-3 interferes with the survival function of IGF-I. Apoptosis was measured in H1299 cells, which were untreated or infected with Ad5CMV or Ad5CMV-BP3 and then allowed to grow in serum-free medium containing 100 ng/ml IGF-I for 3 days. Floating and adherent cells were analyzed using a FACScan flow cytometer (Becton Dickinson, San Jose, Calif.) to determine the percentage of apoptotic cells. Results are expressed relative to the apoptosis of cells incubated in serum-free medium for 3 days. (FIG. 9B) Activated Akt/PKB or MAPKK (MEK)-1 protects H1299 cells from IGFBP-3-induced apoptosis. Induction of apoptosis by 1×10⁴ particles/cell of Ad5CMV-BP3 in H1299 cells transfected with the control pCMV vector or consititutively active Akt (MyrAkt) or consititutively active MAPKK (MEK1/R4F) was analyzed by flow cytometry using APO-BRDU staining. No appreciable change was noted in Ad5CMV-infected H1299 cells transfected with any of these expression vectors. The bar graphs indicate the amounts of apoptosis in H1299 cells transfected with respective expression constructs after the infection of 1×10⁴ particles/cell of Ad5CMV-BP3 or Ad5CMV.

FIGS. 10A-10B—(FIG. 10A) The expression of IGFBP-3 in a panel of NSCLC cell was examined by northern blot analysis using full-length IGFBP-3 or GAPDH cDNA probe as a control. (FIG. 10B) The effect of 5-aza-dC on IGFBP-3 expression was studied by northern blot analysis in NCI-H1299 cells treated with 5′-aza-dC at concentrations of 0.1, 1 and 5μM for 5 days in RPMI1640 supplemented with 2% FCS. The expression of GAPDH was analyzed as a control.

FIGS. 11A-11B—(FIG. 11A) MSP analysis of CpG islands of IGFBP-3 promoter region in NSCLC cell lines as described in Material and Methods. Lanes U, amplification with primers recognizing unmethylated IGFBP-3 alleles; Lanes M, amplification with primers recognizing methylated IGFBP-3 alleles. (FIG. 11B) MSP analysis was performed on stage I NSCLC tumors as described in Materials and Methods. Representative results using tumor tissues from 14 stage I NSCLC patients are shown. Lanes U, amplification with primers recognizing unmethylated IGFBP-3 alleles; Lanes M, amplification with primers recognizing methylated IGFBP-3 alleles. Numbers above each gel identify the primary tumor analyzed. S.M=DNA size marker; U=PCR products with the use of unmethylated-specific primer set; M=PCR™ products with the use of methylated-specific primer set. Among the tumor data shown, methylation was observed in tumors 41, 58, 60, 74, 86, 101, 103, 104, and 106.

FIGS. 12A-12C—Methylation of IGFBP-3 promoter in stage I NSCLC and probability of survival. The Kaplan-Meier method was used to determine the survival probability, and the log-rank test was used to compare the survival curves between groups. (FIG. 12A) Probability of overall survival for patients with methylation of IGFBP-3 (M) versus patients without it (U). At year 5, for the group with methylation of IGFBP-3, the 95% confidence interval (CI) is 27.3%-55.4%; for the group without methylation, the 95% CI is 48.9%-83.7%. (FIG. 12B) Probability of disease-specific survival at various times for patients with methylation of IGFBP-3 versus patients without methylation. At year 5, for the group with methylation of IGFBP-3, the 95% (CI) is 40.0%-70.6%; for the group without methylation, the 95% CI is 74.3%-99.8%. (FIG. 12C) Probability of disease-free survival at various times for patients with methylation of IGFBP-3 versus patients without methylation. At year 5, for the group with methylation of IGFBP-3, the 95% CI is 24.3%-55.0%; for the group without methylation, the 95% CI is 62.3%-93.3%.

FIGS. 13A-13B—Probability of disease-specific and disease-free survival probability at various times for patients with squamous cell carcinoma. (FIG. 13A) Probability of disease-specific survival at various times for patients with squamous cell carcinoma and IGFBP-3 methylation versus patients with squamous cell carcinoma but without methylation. At year 5, for the group with squamous cell carcinoma and IGFBP-3 methylation, the 95% CI is 14.5%-56.2%; for the group with squamous cell carcinoma but without methylation, 95% CI is 1.4%-84.5%. (FIG. 13B) Probability of disease-free survival at various times for patients with squamous cell carcinoma and IGFBP-3 methylation versus patients with squamous cell carcinoma but without methylation. At year 5, for the group with squamous cell carcinoma and IGFBP-3 methylation, the 95% CI is 5.3%-54%; for the group with squamous cell carcinoma and without IGFBP-3 methylation, the 95% CI is 38.7%-94.7%.

FIGS. 14A-14B—(FIG. 14A) IGFBP3 and dnIGFIR inhibit chorioallantoic membrane angiogenesis induced by bFGF. Representative photographs of disks and surrounding CAMs are shown (FIG. 14B).

FIGS. 15A-15B—Lung metastasis model of human lung cancer cells. (FIG. 15A) Nude mice were inoculated intravenously with control, empty adenovirus (AdSCMV) or adenovirus expressing IGFBP3 (AdSCMV-IGFBP3). (FIG. 15B) A similar experiment was conducted as in FIG. 15A using a rat model in which rats were inoculated in the tail vein with control, empty adenovirus (AdSCMV) or adenovirus expressing IGFBP3 (Ad5CMV-IGFBP3).

FIG. 16—Effects of Ad5CMV-BP3 on HNSCC cell lines.

FIG. 17—Ad5CMV-BP3 infection increases IGFBP3 expression in head and neck cancer cell lines.

FIGS. 18A-18B—Effects of deguelin on the proliferation of normal human bronchial epithelial (NHBE), immortalized premalignant (1799 and 1198), malignant HBE (1170-1), and another immortalized HBE cell line, HB56B cells. (FIG. 18A) Normal, 1799, 1198, 1170-1, and HB56B cells were seeded into 96-well culture plates (2×10³ to 5×10³ cells/well) and allowed to adhere overnight. The next day, the cells were treated with various concentrations of deguelin or with 0.1% dimethyl sulfoxide (DMSO) as a control. Cell proliferation was assessed by the MTT assay after 1, 2, or 3 days. Results are expressed as percent cell proliferation relative to the proliferation of DMSO-treated cells (Con). Each bar represents the mean value of six identical wells from a representative single experiment (n=3). Error bars show upper 95% confidence interval. Open bars=untreated control (Con); dotted bars=10⁻⁹ M deguelin; striped bars=10⁻⁸ M deguelin; solid bars=10⁻⁷ M deguelin. **, P<0.001 for cells treated with deguelin relative to control cells for each series of experiments. (FIG. 18B) Effects of deguelin on cell cycle distribution of 1799 cells and NHBE cells. 1799 cells and NHBE cells treated with 0.1% DMSO (Con) or the indicated concentrations of deguelin for 3 days were analyzed for DNA content (propidium iodide uptake) and for percentage of cells in specific phases of the cell cycle (G1, S, and G2/M) by flow cytometry. A representative experiment of two experiments is shown.

FIG. 19—Effect of deguelin on apoptosis in 1799 cells. 1799 and normal human bronchial epithelial (NHBE) cells were treated with deguelin (10⁹ M, 10⁻⁸ M, or 10⁻⁷ M) or dimethyl sulfoxide (DMSO; 0.1%) for 3 days. Cells were processed for apoptosis with the APO-BrdU staining assay. DNA content was determined by uptake of propidium iodide (x-axis). Apoptotic cells were determined by the intensity of fluorescein isothiocyanate (FITC) staining (y-axis). The number of apoptotic cells is represented by the number of FITC-positive cells of the total gated cells. Each value presented is the percentage of apoptotic cells. The percentage of dead cells was determined by flow cytometry analysis of propidium iodide-stained nuclei. Data shown are from a single representative experiment (n=2).

FIG. 20—Effect of deguelin on phosphatidylinositol 3-kinase (PI3K)/Akt pathway in human bronchial epithelial (HBE) cells. The PI3K activity of the immune complex was analyzed. Because the substrate contained a mixture of phophatidylinositols, the PI3K products included mixtures of phosphatidylinositol phosphate (PIP). The data were quantified and expressed as a percentage of control. The percentage of activity was calculated by using the following equation: % activity=A/C×100, where A and C represent the density of the PIP on the phospholipase chromatography plate and the P85α protein band on the Western blot for each time point and control, respectively. The expression levels of the PI3K components p85 α and p110 α for each time point were detected by Western blot analysis.

FIGS. 21A-21B—Effect of deguelin on premalignant human bronchial epithelial (HBE) cells expressing a constitutively active Akt. (FIG. 21A) Control (uninfected [Con]) 1799 cells or 1799 cells infected with Ad5CMV or different concentrations of Ad5CMV-MyrAkt-HA were treated with deguelin (10⁻⁷ M or 10⁻⁶ M) or N-(4-hydroxyphenyl)retinamide (4-HPR) (2×10⁻⁶ M or 4×10⁻⁶ M) for 2 days. Proliferation was measured using the MTT assay. Results are expressed as percent cell proliferation relative to the proliferation of dimethyl sulfoxide (DMSO)-treated uninfected cells. Each bar represents the mean value of six identical wells from a representative single experiment (n=3). Error bars show upper 95% confidence intervals. **, P<0.001 for cells treated with deguelin relative to control cells for each series of experiments. (FIG. 21B) Uninfected control 1799 cells or 1799 cells infected with Ad5CMV (5×10³ viral p/cell) or Ad5CMV-MyrAkt-HA (1×10³or 5×10³ viral p/cell) and treated with DMSO or deguelin (10⁻⁷ M) for 2 days. Cells were processed for apoptosis with the APO-BrdU staining assay. The number of apoptotic cells is represented by the number of fluorescein isothiocyanate (FITC)-positive cells of the total gated cells. Representative data are shown from a single experiment (n=2).

FIG. 22—Effects of deguelin on squamous human bronchial epithelial (HBE) cells. Squamous HBE cells were left uninfected (Con), infected with a control adenovirus (Ad5CMV) at 5×10³ particles per cell (p/cell), or infected with an adenovirus that expresses constitutively active Akt (AdSCMV-MyrAkt-HA) at 1×10³ or 5×10³ p/cell. Cells were then treated with deguelin (10⁻⁷ M or 10⁻⁶ M) for 1 day. Proliferation was measured using the MTT assay. Results are expressed as percent cell proliferation relative to the proliferation of control uninfected cells. Each bar represents the mean value of six identical wells from a representative single experiment (n=3). Error bars show upper 95% confidence intervals. **, P<0.001 for cells treated with deguelin relative to control cells for each series of experiments.

### DESCRIPTION OF ILLUSTRATIVE EMBODIMENTS

I. The Present Invention

A. Cancer Treatment

Previous studies have shown that IGFs are potent mitogens in several NSCLC and SCLC cells (Lee et al., 1996a; Macaulay et al., 1990). Therapeutic strategies designed to interfere with IGF-I-mediated signal transduction, such as soluble IGF-IR, anti-IGF-IR antibody, and an adenovirus vector expressing IGF-1R, show anti-tumor effects of IGF-1R inhibition (Baserga, 1999; Lee et al., 1996a; D'Ambrosio et al., 1996). In addition, IGFBPs play a role in regulating cell growth by competitively binding IGFs and preventing their binding to IGF-IR (Nemunatis et al., 2000; Brodt et al., 2000; Cohick and Clemmons, 1994; Romgnolo et al., 1994; Grill and Cohick, 2000; McCusker et al., 1991).

The inventor has previously demonstrated that adenovirus expressing IGFBP-6 (Ad5CMV-BP6) reduces the growth of NSCLC cells in vitro and in vivo (Sueoka et al., 2000). However, because IGFBP-6 binds with higher affinity to IGF-II than to IGF-I, and because IGFBP-3 is the most abundant IGFBP in human serum, the inventor chose to investigate the effects of IGFBP-3 on the growth of NSCLC cells using a recombinant adenovirus that expresses IGFBP-3 under the control of a CMV promoter (Ad5CMV-BP3).

While IGFs stimulated the growth of a subset of NSCLC cell lines, a result in agreement with previous findings (Zia et al., 1996), Ad5CMV-BP3 inhibited the IGF-I-stimulated growth of these cells. Previous findings have shown an IGF-dependent growth-inhibitory effect of IGFBP-3 in human promyeloid cell line HL60 and breast cancer cells (Conover et al., 1990; Pratt and Pollak, 1994; Grimberg and Cohen, 2000). However, Hochscheid et al. (2000) found that IGFBP-3 induced IGF-independent growth inhibition in a NSCLC cell line, observing that a higher concentration of IGF-I did not influence the proliferation of NSCLC cells stably transfected with IGFBP-3. The difference in these results might have been due to cell-type specificity or different model systems used in the studies. In addition, stable transfection of IGFBP-3 might cause IGF-independent cell growth, and the growth of stably transfected cells could be regulated directly by IGFBP-3.

The crucial roles of IGF-IR in the establishment and maintenance of the transformed phenotype have been underscored (Lee et al., 1996a; Macaulay et al., 1990; D'Ambrosio et al., 1996; Sueoka et al., 2000). In the present study, infection of Ad5CMV-BP3 decreased the clonogenicity of H1299 cells in soft agar by more than 90%, which was comparable to the 84% decrease by an adenovirus expressing antisense IGF-IR (Lee et al., 1996a). Furthermore, the growth of NSCLC tumors established in nude mice was decreased by the injection of Ad5CMV-BP3, indicating that IGFBP-3 overexpression inhibits the growth of NSCLC cells in vitro and in vivo. These dramatic effects of IGFBP-3 could be caused by combined IGF-dependent suppression of IGF-1R signaling and direct IGF-independent effects, possibly mediated via retinoid X receptor (Liu et al., 2000), and indicate a role for IGFBP-3 gene therapy in the control of NSCLC.

The role of IGFBP-3 as an inhibitor of Akt/PKB activation has particular clinical implications, especially in the treatment of NSCLC, where constitutive activation of Akt/PKB occurs at a high frequency (Brognard et al., 2001). The role of Akt/PKB in survival has been demonstrated in studies in which cells were exposed to different apoptotic stimuli such as UV irradiation, growth factor withdrawal, cell-cycle discordance, DNA damage, and TGF-β (Kennedy et al., 1999; Chen et al., 1998; Crowder and Freeman, 1998; Gerber et al., 1998; Hausler et al., 1998; Kulik and Weber, 1998). Manipulating Akt/PKB activity alters the sensitivity of cells to chemotherapy and irradiation; addition of a PI3K inhibitor or transfection of kinase-dead Akt/PKB into cells with high levels of Akt/PKB activity causes dramatic sensitization to these treatments (Brognard et al., 2001). Therefore, in addition to constituting a therapy on its own, targeting Akt/PKB with Ad5CMV-BP3 can enhance the efficacy of chemotherapy and radiation therapy and increase the apoptotic potential of NSCLC cells.

B. Cancer Diagnosis

DNA methylation, the major form of epigenetic information in mammalian cells, has profound effects on the mammalian genome, including transcriptional repression, chromatin structure modulation, X-chromosome inactivation, genomic imprinting, and suppression of the detrimental, effects of repetitive and parasitic DNA sequences on genome integrity (Baylin and Herman, 2000; Jones and Laird, 1999; Robertson and Wolffe, 2000). Genomic methylation patterns are frequently altered in tumor cells with global hypomethylation accompanying region-specific hypermethylation events. The methylation events occur within the CpG islands in specific regions of the promoter of several tumor suppressor genes and lead to a progressive loss of expression of growth inhibitory genes, providing the cells with a growth advantage in a manner akin to deletions or mutations (Robertson, 2001). A recent study using a monoclonal antibody specific for 5-methylcytosine to evaluate the status of global DNA methylation suggests that alteration in DNA methylation is an important epigenetic difference in susceptibility for the development of lung cancer (Piyathilake et al., 2001).

In this study, the inventor demonstrated that the methylation of IGFBP-3 is an important mechanism for silencing IGFBP-3 expression in human NSCLC cell lines. In addition, the inventor observed that the correlation of the methylation changes with clinicopathological characteristics and prognostic factors in a large number of patients with stage I NSCLC. Based on several findings, the inventor hypothesized that methylation occurs in specific CpG islands within the IGFBP-3 promoter, thereby the expression of IGFBP-3 is silenced. First, a subset of NSCLC cell lines have very low IGFBP-3 mRNA and protein level, and mRNA level in these cell lines reflects the protein expression, indicating that transcription is one of major mechanism for the regulation of IGFBP-3 expression in NSCLC cells. Second, aberrant methylation of CpG islands in the promoter region has been associated with transcriptional inactivation of gene expression (Tate and Bird, 1993). Third, structural analysis of IGFBP-3 showed CpG islands spanning the region from −250 to 600 bp relative to the mRNA cap site (Cubbage et al., 1990). Fourth, the expression of IGFBP-3 mRNA was restored in H1299 cells by the treatment of pharmacological demethylating agent, 5-aza-dC.

To confirm this hypothesis, MSP analysis was performed using genomic DNA from NSCLC cells before and after the bisulfite modification. The precise position of methylated CpG sites in the promoter region of IGFBP-3 was determined in H1299 NSCLC cells by sequencing genomic DNA before and after the bisulfite modification. The methylated- and unmethylated-specific primer for MSP reaction was designed based on these data and published results from promoter deletion analysis and luciferase assay showing the minimum promoter region (Walker et al., 2001). The inventor reaffirmed by luciferase assay that this position is a critical site in promoter activity for IGFBP-3 in NSCLC cells, regardless of host-cell methylation status. According to the MSP analysis, 7 of 14 (50%) NSCLC cell lines showed methylation in the IGFBP-3 promoter.

Aberrant promoter methylation has been described for several genes, such as RARβ, TIMP-3, p16^{INK4a}, MGMT, ECAD, p14^{ARF}, and GSTP1, in resected primary NSCLC (Zochbauer-Muller et al., 2001). In colon cancer, the expression of the p16 tumor suppressor gene and the hMLH1 mismatch repair gene was shown to be silenced by DNA methylation (Toyota et al., 1999). These findings indicate that several genes involved in the regulation of tumor progression lose their function through methylation in many cancers (Robertson, 2001). Methylation in cell lines might occur at a greater frequency in case cells with methylated IGFBP-3 are favored in, or arise from the process of, in vitro growth selection.

Hence, the inventor investigated the methylation of IGFBP-3 in 123 resected tissues from primary NSCLC patients. Strikingly, methylation of IGFBP-3 occurred at a greater frequency in our study, indicating that methylation may be a predominant mechanism of IGFBP-3 inactivation in NSCLC. The inventor then investigated the correlation between methylation of IGFBP-3 and the potent clinicopathological characteristics as well as the survival duration of the patients. The inventor noted trends in which later-stage NSCLC show more frequent methylation compared with early-stage NSCLC. Interestingly, the patients with the methylated IGFBP-3 promoter showed significantly poorer disease-free and disease-specific survival probability.

In another study, the inventor therefore focused on a panel of 83 tumors in stage I NSCLC, which allowed determination of the statistically significant correlation between the methylation status of the IGFBP-3 gene and the individual patient prognosis. The inventor found methylation of the IGFBP-3 promoter in 51 of 83 (61.4%) patients who were diagnosed with stage I NSCLC, whereas, no methylation was found in 10 nonmalignant bronchial brush samples from volunteers. Of note, patients with methylation had significantly poorer overall, disease-specific, and disease-free survival probability compared with those without methylation. According to the multivariate analysis, only methylation status was an independent factor that could predict poorer overall, disease-specific, and disease-free survival probability of patients diagnosed with pathologic stage I NSCLC.

The methylation of cancer-related genes has been reported as an indicator of patient prognosis in resected primary NSCLC. Recently, Tang et al. reported that the promoter methylation of DAP-kinase is one indicator of poorer overall and disease-specific survival probability in early-stage NSCLC (Tang et al., 2000) p16 methylation was also an independent risk factor predicting significantly shorter post-surgical survival in patients with stage I adenocarcinoma of lung (Kim et al., 2001). According to their data, the methylation rate of these molecules in primary NSCLC varies from 7 to 44% (Toyota et al., 1999; Merlo et al., 1995; Kashiwabara et al., 1998; Esteller et al., 1998). An analysis of the IGFBP-3 promoter can add to this understanding of survival.

II. IGFBP-3

IGFBP-3, one of the six-membered IGFBP family, regulates IFG-I bioactivity by sequestering IGF-I away from its receptor in the extracellular millieu and thereby inhibiting the mitogenic and anti-apoptotic action of IGF-I (Valentinis et al., 1995; Leal et al., 1997; Liu et al., 2000; Schedlich et al., 2000; Oh et al., 1995; Rozen et al., 1998). The finding of a negative correlation between serum IGFBP-3 levels and cancer risk (Liu et al., 2000) indicates a protective role of IGFBP-3 against the effects of systemic IGF-I. IGFBP-3 also has IGF-I-independent anti-proliferative and pro-apoptotic effects, as shown by the findng that IGFBP-3 overexpression inhibits the growth of fibroblasts that are IGF-I null (Schedlich et al., 2000; Oh et al., 1995). These effects of IGFBP-3 are probably mediated by other cell surface receptors, such as the transforming growth factor (TGF)-β receptor (Rozen et al., 1998). However, the intracellular mechanisms by which IGFBP-3 mediates IGF-I-independent anti-proliferative and pro-apoptotic effects remain largely unknown. It has been demonstrated that IGFBP-3 is tranlocated to the nucleus, where it could exert a direct influence on gene expression (Han t al, 1997; Huynh et al., 1996; Huynh et al., 1998). Thus, nuclear IGFBP-3 may mediate its IGF-independent cellular effects via direct or indirect interaction with growth inhibitory genes or apoptotic genes, or both.

IGFBP-3 gene expression has been shown to be induced by other growth-inhibitory (and apoptosis-inducing) agents such as TGF-β1 (Agarwal et al., 1999), TNF-α (Buckbinder et al., 1995), retinoic acid (Cohick and Clemmons, 1994), anti-estrogen ICI 182780 (Romgnolo et al., 1994), vitamin D and its analogues EB1089 and CB1093) (Grill and Cohick, 2000; McCusker et al., 1991) and transcription factor 53 (Campbell et al., 1998). These findings raise the possibility that these agents mediate their cellular effects through IGFBP-3. IGFBP-3 also has the ability to potentiate IGF-I bioactivity in several different cell types (Lee et al., 1998a; Sueoka et al., 2000; Lee et al., 1998b). Although the mechanism is large unknown, enhancement of IGF-I's actions is though to occur following IGFBP-3's association with the cell membrane, and thereby facilitating IGF-I's binding to its receptor (Zhang et al., 1994). Alternatively, surface associated IGFBP-3 may be targeted for proteolysis into fragments that have reduced IGF-I affinity (Baserga, 1999). Whether IGFBP-3 assumes an inhibitory or enhancing role may depend on the cell type and the compound that induces its expression.

A. IGFBP-3 Structure

Analysis of the human IGFBP-3 cDNA (Wood et al., 1988) predicts a core protein of 264 amino acids with a molecular mass of about 29 kDa. The amino acid sequence predicts three potential N-glycosylation sites (Asn-X-Ser/Thr) located at Asn⁸⁹, Asn¹⁰⁹ and Asn¹⁷² (sites 1, 2 and 3 respectively) in the central region, which is not conserved among IGFBPs. Native hIGFBP-3 is usually found as a characteristic doublet of about 40-45 kDa, from both cellular and plasma sources. The primary sequence of human IGFBP-3 is set forth in SEQ ID NO:1. See also Accession Nos. M35878, J05537, J05538, M35879, M35880, M35881, M35882, M35883, M35884, M35885, M35886, M36121, and M36122.

In addition to an entire IGFBP-3 molecule, the present invention also relates to fragments of the polypeptides that may or may not retain various of the functions described below Fragments, including the N-terminus of the molecule may be generated by genetic engineering of translation stop sites within the coding region (discussed below). Alternatively, treatment of the IGFBP-3 with proteolytic enzymes, known as proteases, can produce a variety of N-terminal, C-terminal and internal fragments. Examples of fragments may include contiguous residues of SEQ ID NO:1 of 6, 7, 8, 9, 10, 11, 12, 13, 14, 15, 16, 17, 18, 19, 20, 21, 22, 23, 24, 25, 30, 35, 40, 45, 50, 55, 60, 65, 75, 80, 85, 90, 95, 100, 200, 300, 400 or more amino acids in length. These fragments may be purified according to known methods, such as precipitation (e.g., ammonium sulfate), HPLC, ion exchange chromatography, affinity chromatography (including immunoaffinity chromatography) or various size separations (sedimentation, gel electrophoresis, gel filtration).

B. Variants of IGFBP-3

Amino acid sequence variants of the polypeptide can be substitutional, insertional or deletion variants. Deletion variants lack one or more residues of the native protein which are not essential for function or immunogenic activity, and are exemplified by the variants lacking a transmembrane sequence described above. Another common type of deletion variant is one lacking secretory signal sequences or signal sequences directing a protein to bind to a particular part of a cell. Insertional mutants typically involve the addition of material at a non-terminal point in the polypeptide. This may include the insertion of an immunoreactive epitope or simply a single residue. Terminal additions, called fusion proteins, are discussed below.

Substitutional variants typically contain the exchange of one amino acid for another at one or more sites within the protein, and may be designed to modulate one or more properties of the polypeptide, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The following is a discussion based upon changing of the amino acids of a protein to create an equivalent, or even an improved, second-generation molecule. For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and its underlying DNA coding sequence, and nevertheless obtain a protein with like properties. It is thus contemplated by the inventor that various changes may be made in the DNA sequences of genes without appreciable loss of their biological utility or activity, as discussed below. Table 1 shows the codons that encode particular amino acids.

In making such changes, the hydropathic index of amino acids may be considered. The importance of the hydropathic amino acid index in conferring interactive biologic function on a protein is generally understood in the art (Kyte and Doolittle, 1982). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte and Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtain a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

Another embodiment for the preparation of polypeptides according to the invention is the use of peptide mimetics. Mimetics are peptide-containing molecules that mimic elements of protein secondary structure (Johnson et al, 1993). The underlying rationale behind the use of peptide mimetics is that the peptide backbone of proteins exists chiefly to orient amino acid side chains in such a way as to facilitate molecular interactions, such as those of antibody and antigen. A peptide mimetic is expected to permit molecular interactions similar to the natural molecule. These principles may be used, in conjunction with the principles outline above, to engineer second generation molecules having many of the natural properties of IGFBP-3, but with altered and even improved characteristics.

C. Purification of Proteins

It will be desirable to purify IGFBP-3 or variants thereof. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present invention concern the purification, and in particular embodiments, the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

D. Synthetic Peptides

The present invention also describes smaller IGFBP-3-related peptides for use in various embodiments of the present invention. Because of their relatively small size, the peptides of the invention can also be synthesized in solution or on a solid support in accordance with conventional techniques. Various automatic synthesizers are commercially available and can be used in accordance with known protocols. See, for example, Stewart and Young (1984); Tam et al. (1983); Merrifield (1986); and Barany and Merrifield (1979), each incorporated herein by reference. Short peptide sequences, or libraries of overlapping peptides, usually from about 6 up to about 35 to 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

E. Antigen Compositions

The present invention also provides for the use of IGFBP-3 proteins or peptides as antigens for the immunization of animals relating to the production of antibodies. It is envisioned that IGFBP-3, or portions thereof, will be coupled, bonded, bound, conjugated or chemically-linked to one or more agents via linkers, polylinkers or derivatized amino acids. This may be performed such that a bispecific or multivalent composition or vaccine is produced. It is further envisioned that the methods used in the preparation of these compositions will be familiar to those of skill in the art and should be suitable for administration to animals, i.e., pharmaceutically acceptable. Preferred agents are the carriers are keyhole limpet hemocyannin (KLH) or bovine serum albumin (BSA).

III. Nucleic Acids

The present invention also provides, in another embodiment, genes encoding IGFBP-3. See, for example, SEQ ID NO:2. In addition, it should be clear that the present invention is not limited to the specific nucleic acids disclosed herein. As discussed below, “an IGFBP-3 gene” may contain a variety of different bases and yet still produce a corresponding polypeptide that is functionally indistinguishable, and in some cases structurally, from the human and mouse genes disclosed herein.

Similarly, any reference to a nucleic acid should be read as encompassing a host cell containing that nucleic acid and, in some cases, capable of expressing the product of that nucleic acid. In addition to therapeutic considerations, cells expressing nucleic acids of the present invention may prove useful in the context of screening for agents that induce, repress, inhibit, augment, interfere with, block, abrogate, stimulate or enhance the activity of IGFBP-3.

A. Nucleic Acids Encoding IGFBP-3

Nucleic acids according to the present invention may encode an entire IGFBP-3 gene, a domain of IGFBP-3, or any other fragment of IGFBP-3 as set forth herein. The nucleic acid may be derived from genomic DNA, i.e., cloned directly from the genome of a particular organism. In preferred embodiments, however, the nucleic acid would comprise complementary DNA (cDNA). Also contemplated is a cDNA plus a natural intron or an intron derived from another gene; such engineered molecules are sometime referred to as “mini-genes.” At a minimum, these and other nucleic acids of the present invention may be used as molecular weight standards in, for example, gel electrophoresis.

The term “cDNA” is intended to refer to DNA prepared using messenger RNA (mRNA) as template. The advantage of using a cDNA, as opposed to genomic DNA or DNA polymerized from a genomic, non- or partially-processed RNA template, is that the cDNA primarily contains coding sequences of the corresponding protein. There may be times when the full or partial genomic sequence is preferred, such as where the non-coding regions are required for optimal expression or where non-coding regions such as introns are to be targeted in an antisense strategy.

It also is contemplated that a given IGFBP-3 from a given species may be represented by natural variants that have slightly different nucleic acid sequences but, nonetheless, encode the same protein (see Table 1 below).

As used in this application, the term “a nucleic acid encoding a IGFBP-3” refers to a nucleic acid molecule that has been isolated free of total cellular nucleic acid. In preferred embodiments, the invention concerns a nucleic acid sequence essentially as set forth in SEQ ID NO: 2. The term “as set forth in SEQ ID NO:2” means that the nucleic acid sequence substantially corresponds to a portion of SEQ ID NO:2. The term “functionally equivalent codon” is used herein to refer to codons that encode the same amino acid, such as the six codons for arginine or serine (Table 1, below), and also refers to codons that encode biologically equivalent amino acids, as discussed in the following pages.

TABLE 1

| | |
|---|---|
| Amino Acids | Codon |

| | | | |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

Allowing for the degeneracy of the genetic code, sequences that have at least about 50%, usually at least about 60%, more usually about 70%, most usually about 80%, preferably at least about 90% and most preferably about 95% of nucleotides that are identical to the nucleotides of SEQ ID NO:2 are contemplated. Sequences that are essentially the same as those set forth in SEQ ID NO:2 may also be functionally defined as sequences that are capable of hybridizing to a nucleic acid segment containing the complement of SEQ ID NO:2 under standard conditions.

The DNA segments of the present invention include those encoding biologically functional equivalent IGFBP-3 proteins and peptides, as described above. Such sequences may arise as a consequence of codon redundancy and amino acid functional equivalency that are known to occur naturally within nucleic acid sequences and the proteins thus encoded. Alternatively, functionally equivalent proteins or peptides may be created via the application of recombinant DNA technology, in which changes in the protein structure may be engineered, based on considerations of the properties of the amino acids being exchanged. Changes designed by man may be introduced through the application of site-directed mutagenesis techniques or may be introduced randomly and screened later for the desired function, as described below.

B. Oligonucleotide Probes and Primers

Naturally, the present invention also encompasses DNA segments that are complementary, or essentially complementary, to the sequence set forth in SEQ ID NO:2. Nucleic acid sequences that are “complementary” are those that are capable of base-pairing according to the standard Watson-Crick complementary rules. As used herein, the term “complementary sequences” means nucleic acid sequences that are substantially complementary, as may be assessed by the same nucleotide comparison set forth above, or as defined as being capable of hybridizing to the nucleic acid segment of SEQ ID NO:2 under relatively stringent conditions such as those described herein. Such sequences may encode entire IGFBP-3 proteins or functional or non-functional fragments thereof.

Alternatively, the hybridizing segments may be shorter oligonucleotides. Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides encoding 250, 500, 1000, 1212, 1500, 2000, 2500, 3000 or 5000 bases and longer are contemplated as well. Such oligonucleotides will find use, for example, as probes in Southern and Northern blots and as primers in amplification reactions.

Suitable hybridization conditions will be well known to those of skill in the art. In certain applications, for example, substitution of amino acids by site-directed mutagenesis, it is appreciated that lower stringency conditions are required. Under these conditions, hybridization may occur even though the sequences of probe and target strand are not perfectly complementary, but are mismatched at one or more positions. Conditions may be rendered less stringent by increasing salt concentration and decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Thus, hybridization conditions can be readily manipulated, and thus will generally be a method of choice depending on the desired results.

In other embodiments, hybridization may be achieved under conditions of, for example, 50 mM Tris-HCl (pH 8.3), 75 mM KCl, 3 mM MgCl₂, 10 mM dithiothreitol, at temperatures between approximately 20° C. to about 37° C. Other hybridization conditions utilized could include approximately 10 mM Tris-HCl (pH 8.3), 50 mM KCl, 1.5 μM MgCl₂, at temperatures ranging from approximately 40° C. to about 72° C. Formamide and SDS also may be used to alter the hybridization conditions.

One method of using probes and primers of the present invention is in the search for genes related to IGFBP-3 or, more particularly, homologs of IGFBP-3 from other species. Normally, the target DNA will be a genomic or cDNA library, although screening may involve analysis of RNA molecules. By varying the stringency of hybridization, and the region of the probe, different degrees of homology may be discovered.

Another way of exploiting probes and primers of the present invention is in site-directed, or site-specific mutagenesis. Site-specific mutagenesis is a technique useful in the preparation of individual peptides, or biologically functional equivalent proteins or peptides, through specific mutagenesis of the underlying DNA. The technique further provides a ready ability to prepare and test sequence variants, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

The technique typically employs a bacteriophage vector that exists in both a single-stranded and double-stranded form. Typical vectors useful in site-directed mutagenesis include vectors such as the M13 phage. These phage vectors are commercially available and their use is generally well known to those skilled in the art. Double stranded plasmids are also routinely employed in site directed mutagenesis, which eliminates the step of transferring the gene of interest from a phage to a plasmid.

In general, site-directed mutagenesis is performed by first obtaining a single-stranded vector, or melting of two strands of a double-stranded vector which includes within its sequence a DNA sequence encoding the desired protein. An oligonucleotide primer bearing the desired mutated sequence is synthetically prepared. This primer is then annealed with the single-stranded DNA preparation, taking into account the degree of mismatch when selecting hybridization conditions, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

The preparation of sequence variants of the selected gene using site-directed mutagenesis is provided as a means of producing potentially useful species and is not meant to be limiting, as there are other ways in which sequence variants of genes may be obtained. For example, recombinant vectors encoding the desired gene may be treated with mutagenic agents, such as hydroxylamine, to obtain sequence variants.

C. Antisense Constructs

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complementarity to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme; see below) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

D. Ribozymes

Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme.

E. Vectors

Within certain embodiments expression vectors are employed to express a IGFBP-3 polypeptide product, which can then be purified and, for example, be used to vaccinate animals to generate antisera or monoclonal antibody with which further studies may be conducted. In other embodiments, the expression vectors are used in gene therapy. Expression requires that appropriate signals be provided in the vectors, and which include various regulatory elements, such as enhancers/promoters from both viral and mammalian sources that drive expression of the genes of interest in host cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

(i) Regulatory Elements

Throughout this application, the term “expression construct” is meant to include any type of genetic construct containing a nucleic acid coding for a gene product in which part or all of the nucleic acid encoding sequence is capable of being transcribed. The transcript may be translated into a protein, but it need not be. In certain embodiments, expression includes both transcription of a gene and translation of mRNA into a gene product. In other embodiments, expression only includes transcription of the nucleic acid encoding a gene of interest.

In preferred embodiments, the nucleic acid encoding a gene product is under transcriptional control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In various embodiments, the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose.

By employing a promoter with well-known properties, the level and pattern of expression of the protein of interest following transfection or transformation can be optimized. Further, selection of a promoter that is regulated in response to specific physiologic signals can permit inducible expression of the gene product. Tables 2 and 3 list several regulatory elements that may be employed, in the context of the present invention, to regulate the expression of the gene of interest. This list is not intended to be exhaustive of all the possible elements involved in the promotion of gene expression but, merely, to be exemplary thereof.

Enhancers are genetic elements that increase transcription from a promoter located at a distant position on the same molecule of DNA. Enhancers are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of viral promoters, cellular promoters/enhancers and inducible promoters/enhancers that could be used in combination with the nucleic acid encoding a gene of interest in an expression construct (Table 2 and Table 3). Additionally, any other promoter/enhancer combination (for example, as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

TABLE 2
Promoter and/or Enhancer

| Promoter/Enhancer | References |
|---|---|
| Immunoglobulin Heavy Chain | Banerji et al., 1983; Gilles et al., 1983; Grosschedl |
| | et al., 1985; Atchison et al., 1986, 1987; Imler |
| | et al., 1987; Weinberger et al., 1984; Kiledjian |
| | et al., 1988; Porton et al.; 1990 |
| Immunoglobulin Light Chain | Queen et al., 1983; Picard et al., 1984 |
| T-Cell Receptor | Luria et al., 1987; Winoto et al., 1989; Redondo |
| | et al.; 1990 |
| HLA DQ a and/or DQ β | Sullivan et al., 1987 |
| β-Interferon | Goodbourn et al., 1986; Fujita et al., 1987; |
| | Goodbourn et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson |
| | et al., 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987a |
| Albumin | Pinkert et al., 1987; Tronche et al., 1989, 1990 |
| α-Fetoprotein | Godbout et al., 1988; Campere et al., 1989 |
| t-Globin | Bodine et al., 1987; Perez-Stable et al., 1990 |
| β-Globin | Trudel et al., 1987 |
| c-fos | Cohen et al., 1987 |
| c-HA-ras | Treisman, 1986; Deschamps et al., 1985 |
| Insulin | Edlund et al., 1985 |
| Neural Cell Adhesion Molecule | Hirsch et al., 1990 |
| (NCAM) | |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et |
| | al., 1985; Firak et al., 1986; Herr et al., 1986; |
| | Imbra et al., 1986; Kadesch et al;, 1986; Wang et |
| | al., 1986; Ondek et al., 1987; Kuhl et al., 1987; |
| | Schaffner et al., 1988 |
| Polyoma | Swartzendruber et al., 1975; Vasseur et al., 1980; |
| | Katinka et al., 1980, 1981; Tyndell et al., 1981; |
| | Dandolo et al., 1983; de Villiers et al., 1984; Hen |
| | et al., 1986; Satake et al., 1988; Campbell and/or |
| | Villarreal, 1988 |
| Retroviruses | Kriegler et al., 1982, 1983; Levinson et al., 1982; |
| | Kriegler et al., 1983, 1984a, b, 1988; Bosze et al., |
| | 1986; Miksicek et al., 1986; Celander et al., 1987; |
| | Thiesen et al., 1988; Celander et al., 1988; Choi |
| | et al., 1988; Reisman et al., 1989 |
| Papilloma Virus | Campo et al., 1983; Lusky et al., 1983; Spandidos |
| | and/or Wilkie, 1983; Spalholz et al., 1985; Lusky |
| | et al., 1986; Cripe et al., 1987; Gloss et al., 1987; |
| | Hirochika et al., 1987; Stephens et al., 1987 |
| Hepatitis B Virus | Bulla et al., 1986; Jameel et al., 1986; Shaul et al., |
| | 1987; Spandau et al., 1988; Vannice et al., 1988 |
| Human Immunodeficiency Virus | Muesing et al., 1987; Hauber et al., 1988; |
| | Jakobovits et al., 1988; Feng et al., 1988; Takebe |
| | et al., 1988; Rosen et al., 1988; Berkhout et al., |
| | 1989; Laspia et al., 1989; Sharp et al., 1989; |
| | Braddock et al., 1989 |
| Cytomegalovirus (CMV) | Weber et al., 1984; Boshart et al., 1985; Foecking |
| | et al., 1986 |
| Gibbon Ape Leukemia Virus | Holbrook et al., 1987; Quinn et al., 1989 |

TABLE 3
Inducible Elements

| Element | Inducer | References |
|---|---|---|
| MT II | Phorbol Ester (TFA) | Palmiter et al., 1982; |
| | Heavy metals | Haslinger et al., 1985; |
| | | Searle et al., 1985; Stuart |
| | | et al., 1985; Imagawa |
| | | et al., 1987, Karin et al., |
| | | 1987; Angel et al., 1987b; |
| | | McNeall et al., 1989 |
| MMTV (mouse | Glucocorticoids | Huang et al., 1981; Lee |
| mammary tumor | | et al., 1981; Majors et al., |
| virus) | | 1983; Chandler et al., |
| | | 1983; Lee et al., 1984; |
| | | Ponta et al., 1985; Sakai |
| | | et al., 1988 |
| β-Interferon | poly(rI)x | Tavernier et al., 1983 |
| | poly(rc) | |
| Adenovirus 5 E2 | ElA | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I | Interferon | Blanar et al., 1989 |
| Gene H-2κb | | |
| HSP70 | ElA, SV40 Large T | Taylor et al., 1989, 1990a, |
| | Antigen | 1990b |
| Proliferin | Phorbol Ester-TPA | Mordacq et al., 1989 |
| Tumor Necrosis | PMA | Hensel et al., 1989 |
| Factor | | |
| Thyroid Stimulating | Thyroid Hormone | Chatterjee et al., 1989 |
| Hormone α Gene | | |

Where a cDNA insert is employed, one will typically desire to include a polyadenylation signal to effect proper polyadenylation of the gene transcript. The nature of the polyadenylation signal is not believed to be crucial to the successful practice of the invention, and any such sequence may be employed such as human growth hormone and SV40 polyadenylation signals. Also contemplated as an element of the expression cassette is a terminator. These elements can serve to enhance message levels and to minimize read through from the cassette into other sequences.

(ii) Selectable Markers

In certain embodiments of the invention, the cells contain nucleic acid constructs of the present invention, a cell may be identified in vitro or in vivo by including a marker in the expression construct. Such markers would confer an identifiable change to the cell permitting easy identification of cells containing the expression construct. Usually the inclusion of a drug selection marker aids in cloning and in the selection of transformants, for example, genes that confer resistance to neomycin, puromycin, hygromycin, DHFR, GPT, zeocin and histidinol are useful selectable markers. Alternatively, enzymes such as herpes simplex virus thymidine kinase (tk) or chloramphenicol acetyltransferase (CAT) may be employed. Immunologic markers also can be employed. The selectable marker employed is not believed to be important, so long as it is capable of being expressed simultaneously with the nucleic acid encoding a gene product. Further examples of selectable markers are well known to one of skill in the art.

(iii) Multigene Constructs and IRES

In certain embodiments of the invention, the use of internal ribosome binding sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picanovirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message.

Any heterologous open reading frame can be linked to IRES elements. This includes genes for secreted proteins, multi-subunit proteins, encoded by independent genes, intracellular or membrane-bound proteins and selectable markers. In this way, expression of several proteins can be simultaneously engineered into a cell with a single construct and a single selectable marker.

IV. Methods of Gene Transfer

A. Viral Methods

There are a number of ways in which expression constructs may be introduced into cells. In certain embodiments of the invention, a vector (also referred to herein as a gene delivery vector) is employed to deliver the expression construct. By way of illustration, in some embodiments, the vector comprises a virus or engineered construct derived from a viral genome. The ability of certain viruses to enter cells via receptor-mediated endocytosis, to integrate into host cell genome and express viral genes stably and efficiently have made them attractive candidates for the transfer of foreign genes into mammalian cells (Ridgeway, 1988; Nicolas and Rubenstein, 1988; Baichwal and Sugden, 1986; Temin, 1986). The first viruses used as gene delivery vectors were DNA viruses including the papovaviruses (simian virus 40, bovine papilloma virus, and polyoma) (Ridgeway, 1988; Baichwal and Sugden, 1986). Generally, these have a relatively low capacity for foreign DNA sequences and have a restricted host spectrum. They can accommodate only up to 8 kb of foreign genetic material but can be readily introduced in a variety of cell lines and laboratory animals (Nicolas and Rubenstein, 1988; Temin, 1986). Where viral vectors are employed to deliver the gene or genes of interest, it is generally preferred that they be replication-defective, for example as known to those of skill in the art and as described further herein below.

One of the methods for in vivo delivery of expression constructs involves the use of an adenovirus expression vector. “Adenovirus expression vector” is meant to include those constructs containing adenovirus sequences sufficient to (a) support packaging of the construct and (b) to express a polynucleotide that has been cloned therein. In this context, expression does not require that the gene product be synthesized.

In particular embodiments, the expression vector comprises a genetically engineered form of adenovirus. Knowledge of the genetic organization of adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage and are able to infect non-dividing cells such as, for example, cardiomyocytes. So far, adenoviral infection appears to be linked only to mild disease such as acute respiratory disease in humans.

Adenovirus is particularly suitable for use as a gene delivery vector because of its mid-sized genome, ease of manipulation, high titer, wide target cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is important to minimize this possibility by, for example, reducing or eliminating adnoviral sequence overlaps within the system and/or to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the E3 or both regions (Graham and Prevec, 1991). In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and E3 regions, the maximum capacity of such adenovirus vectors is about 7.5 kb, or about 15% of the total length of the vector. Additionally, modified adenoviral vectors are now available which have an even greater capacity to carry foreign DNA.

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, a preferred helper cell line is 293.

Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be selected from any of the 42 different known serotypes or subgroups A-F. Adenovirus type 5 of subgroup C is a preferred starting material for obtaining a replication-defective adenovirus vector for use in the present invention. This is, in part, because Adenovirus type 5 is a human adenovirus about which a great deal of biochemical and genetic information is known, and it has historically been used for most constructions employing adenovirus as a vector.

As stated above, a preferred adenoviral vector according to the present invention lacks an adenovirus E1 region and thus, is replication. Typically, it is most convenient to introduce the polynucleotide encoding the gene of interest at the position from which the E1-coding sequences have been removed. However, the position of insertion of the construct within the adenovirus sequences is not critical to the invention. Further, other adenoviral sequences may be deleted and/or inactivated in addition to or in lieu of the E1 region. For example, the E2 and E4 regions are both necessary for adenoviral replication and thus may be modified to render an adenovirus vector replication-defective, in which case a helper cell line or helper virus complex may employed to provide such deleted/inactivated genes in trans. The polynucleotide encoding the gene of interest may alternatively be inserted in lieu of a deleted E3 region such as in E3 replacement vectors as described by Karlsson et al. (1986), or in a deleted E4 region where a helper cell line or helper virus complements the E4 defect. Other modifications are known to those of skill in the art and are likewise contemplated herein.

Adenovirus is easy to grow and manipulate and exhibits broad host range in vitro and in vivo. This group of viruses can be obtained in high titers, e.g., 10⁹⁻¹⁰¹² plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include administration via intracoronary catheter into one or more coronary arteries of the heart (Hammond, et al., U.S. Pat. Nos. 5,792,453 and 6,100,242) trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

One approach designed to allow specific targeting of retrovirus vectors was developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact-sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and -Sugden, 1986; Hermonat and Muzycska, 1984) and herpesviruses may be employed. They offer several attractive features for various mammalian cells (Friedmann, 1989; Ridgeway, 1988; Baichwal and Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al., recently introduced the chloramphenicol acetyltransferase (CAT) gene into duck hepatitis B virus genome in the place of the polymerase, surface, and pre-surface coding sequences. It was co-transfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

B. Non-Viral Methods

Several non-viral gene delivery vectors for the transfer of expression constructs into mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984), direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment of the invention, the expression vector may simply consist of naked recombinant DNA or plasmids comprising the expression construct. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Neshif (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

In still another embodiment of the invention, transferring of a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

In a further embodiment of the invention, the expression construct may be entrapped in a liposome, another non-viral gene delivery vector. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

Liposome-mediated nucleic acid delivery and expression of foreign DNA in vitro has been very successful. Wong et al., (1980) demonstrated the feasibility of liposome-mediated delivery and expression of foreign DNA in cultured chick embryo, HeLa and hepatoma cells. Nicolau et al., (1987) accomplished successful liposome-mediated gene transfer in rats after intravenous injection.

In certain embodiments of the invention, the liposome may be complexed with a hemagglutinating virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific (Wu and Wu, 1993).

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). Recently, a synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al., (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type by any number of receptor-ligand systems with or without liposomes. For example, epidermal growth factor (EGF) may be used as the receptor for mediated delivery of a nucleic acid into cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In certain embodiments, gene transfer may more easily be performed under ex vivo conditions. Ex vivo gene therapy refers to the isolation of cells from an animal, the delivery of a nucleic acid into the cells in vitro, and then the return of the modified cells back into an animal. This may involve the surgical removal of tissue/organs from an animal or the primary culture of cells and tissues.

C. Aerosolized Method

In some aspects of the present invention, the inventor provides an aerosolized delivery approach for gene delivery in treating lung cancer. Aerosol delivery can reach a large surface area of the bronchial epithelium, does not carry the risks associated with intrathoracic injections, and avoids the toxicities associated with systemic administration. Aerosolized delivery of adenoviral and retroviral vectors has achieved gene transfer to lung cancers in animal models, but this approach is limited by variable viral receptor expression in lung cancer cells, and administration of viral vectors to the lung induces antiviral immune responses that reduce gene transfer.

Thus, in the present invention, a novel aerosolized treatment strategy is contemplated to enhance the efficacy of treatment of lung cancer. This strategy employs delivery of recombinant adenoviral vectors incorporated into calcium phosphate precipitates, a modification that enhances adenoviral gene delivery in airway epithelia, which lack the receptor activity to bind adenovirus fiber protein. For example, the inventor has achieved gene transfer in lung tumors that arise in K-rasLA1 mice, which express mutant K-ras through stochastic activation of a latent allele. Aerosolized delivery of an adenovirus expressing dominant-negative mutant MKK4, that contains a lysine 129 (KR) mutation in the ATP binding region, inhibited Ras-dependent signaling in the lungs of K-rasLA1 mice. Thus the inventor provides aerosolized delivery of calcium phosphate-precipitated adenoviral vectors that is selcetive at the molecular level, as an effective means of gene transfer to lung tumors that may be used with the present invention in treating lung cancer. This technique is useful in the following ways: (a) it can be used in animal models of human lung cancer to investigate, in an in vivo setting, the role of specific intracellular pathways in lung tumorigenesis; and (b) it can be implemented in a clinical setting for the delivery of genes with anticancer activity to lung cancer patients.

V. Routes of Administration and Pharmaceutical Formulations

Where clinical applications are contemplated, it will be necessary to prepare pharmaceutical compositions—polypeptides, expression vectors, virus stocks and drugs—in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifingal agents, isotonic and absorption delaying agents and the like. The use of such media and agents for pharmaceutically active substances is well know in the art. Except insofar as any conventional media or agent is incompatible with the vectors or cells of the present invention, its use in therapeutic compositions is contemplated. Supplementary active ingredients also can be incorporated into the compositions.

The active compositions of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by intratumoral, intradermal, subcutaneous, intramuscular, intraperitoneal, intravascular or intravenous injection. Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifingal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

VI. Combination Therapies

In order to increase the effectiveness of IGFBP-3 polypeptide, or expression construct coding therefor, it may be desirable to combine these compositions with other agents effective in the treatment of hyperproliferative disease, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with gene therapy. In the context of the present invention, it is contemplated that IGFBP-3 therapy could be used in conjunction with chemotherapeutic, radiotherapeutic, or imnunotherapeutic intervention, in addition to other pro-apoptotic or cell cycle regulating agents.

Alternatively, the IGFBP-3 therapy may precede or follow the other agent treatment by intervals ranging from minutes to weeks. In embodiments where the other agent and polypeptide/expression construct are applied separately to the cell, one would generally ensure that a significant period of time did not expire between the time of each delivery, such that the agent and polypeptide/expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, IGFBP-3 therapy is “A” and the secondary agent, such as radio- or chemotherapy, is “B”:

A/B/A B/A/B B/B/A A/A/B A/B/B B/A/A A/B/B/B
B/A/B/B B/B/B/A B/B/A/B A/A/B/B A/B/A/B A/B/B/A
B/B/A/A B/A/B/A B/A/A/B A/A/A/B B/A/A/A A/B/A/A
A/A/B/A

Administration of the therapeutic polypeptides/expression constructs of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described hyperproliferative cell therapy.

a. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

b. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

c. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with IGFBP-3 therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

d. Genes

In yet another embodiment, the secondary treatment is a secondary gene therapy in which a second therapeutic polynucleotide is administered before, after, or at the same time a first therapeutic polynucleotide encoding all of part of an IGFBP-3 polypeptide. Delivery of a vector encoding IGFBP-3 in conduction with a second vector encoding one of the following gene products will have a combined anti-hyperproliferative effect on target tissues. Alternatively, a single vector encoding both genes may be used. A variety of proteins are encompassed within the invention, some of which are described below.

### i. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

### ii. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein p16 also is known to regulate the function of CDK6.

p16^{INK4} belongs to a newly described class of CDK-inhibitory proteins that also includes p16^{B}, p19, p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Kamb et al., 1994; Okamoto et al., 1994; Nobori et al., 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300; genes involved in angiogenesis (e.g., VEGF, FGF, thrombospondin, BAI-1, GDAIF, or their receptors) and MCC.

### iii. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., BCl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri).

e. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

f. Other Agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adehesion, or agents that increase the sensitivity of the hyperproliferative cells to apoptotic inducers immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abililties of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adehesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

VII. Diagnosing and Predicting Cancer With IGFBP-3

As discussed above, the present invention also addresses the diagnostic potential of IGFBP-3 with respect to cancer. In particular, the inventor has determined that IGFBP-3 may well provide important information on the survival of a cancer patient, such as a lung cancer or NSCLC patient. Various methods to carry out this embodiment are contemplated, as discussed below.

A. Antibodies and Immunoassay

In another aspect, the present invention contemplates an antibody that is immunoreactive with a IGFBP-3 molecule of the present invention, or any portion thereof. An antibody can be a polyclonal or a monoclonal antibody. In a preferred embodiment, an antibody is a monoclonal antibody. Means for preparing and characterizing antibodies are well known in the art (see, e.g., Harlow and Lane, 1988).

Briefly, a polyclonal antibody is prepared by immunizing an animal with an immunogen comprising a polypeptide of the present invention and collecting antisera from that immunized animal. A wide range of animal species can be used for the production of antisera. Typically an animal used for production of anti-antisera is a non-human animal including rabbits, mice, rats, hamsters, pigs or horses. Because of the relatively large blood volume of rabbits, a rabbit is a preferred choice for production of polyclonal antibodies.

Antibodies, both polyclonal and monoclonal, specific for isoforms of antigen may be prepared using conventional immunization techniques, as will be generally known to those of skill in the art. A composition containing antigenic epitopes of the compounds of the present invention can be used to immunize one or more experimental animals, such as a rabbit or mouse, which will then proceed to produce specific antibodies against the compounds of the present invention. Polyclonal antisera may be obtained, after allowing time for antibody generation, simply by bleeding the animal and preparing serum samples from the whole blood.

It is proposed that the monoclonal antibodies of the present invention will find useful application in standard immunochemical procedures, such as ELISA and Western blot methods and in immunohistochemical procedures such as tissue staining, as well as in other procedures which may utilize antibodies specific to IGFBP-3-related antigen epitopes. Additionally, it is proposed that monoclonal antibodies specific to the particular IGFBP-3 of different species may be utilized in other useful applications Means for preparing and characterizing antibodies are well known in the art (see, e.g., Harlow and Lane, 1988; incorporated herein by reference). More specific examples of monoclonal antibody preparation are given in the examples below.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimide and bis-biazotized benzidine.

As also is well known in the art, the immunogenicity of a particular immunogen composition can be enhanced by the use of non-specific stimulators of the immune response, known as adjuvants. Exemplary and preferred adjuvants include complete: Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection may also be given. The process of boosting and titering is repeated until a suitable titer is achieved. When a desired level of immunogenicity is obtained, the immunized animal can be bled and the serum isolated and stored, and/or the animal can be used to generate mAbs.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified IGFBP-3 protein, polypeptide or peptide or cell expressing high levels of IGFBP-3. The immunizing composition is administered in a manner effective to stimulate antibody producing cells. Rodents such as mice and rats are preferred animals, however, the use of rabbit, sheep frog cells is also possible. The use of rats may provide certain advantages (Goding, 1986), but mice are preferred, with the BALB/c mouse being most preferred as this is most routinely used and generally gives a higher percentage of stable fusions.

Following immunization, somatic cells with the potential for producing antibodies, specifically B-lymphocytes (B-cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, 1986; Campbell, 1984). For example, where the immunized animal is a mouse, one may use P3-X63/Ag8, P3-X63-Ag8.653, NS1/1.Ag 41, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with cell fusions.

Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Sendai virus have been described (Kohler and Milstein, 1975; 1976), and those using polyethylene glycol (PEG), such as 37% (v/v) PEG, by Gefter et al., (1977). The use of electrically induced fusion methods is also appropriate (Goding, 1986).

Fusion procedures usually produce viable hybrids at low frequencies, around 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B-cells can operate this pathway, but they have a limited life span in culture and generally die within about two weeks. Therefore, the only cells that can survive in the selective media are those hybrids formed from myeloma and B-cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide mAbs. The cell lines may be exploited for mAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they can be readily obtained in high concentrations mAbs produced by either means may be further purified, if desired, using filtration, centrifugation and various chromatographic methods such as HPLC or affinity chromatography.

Antibodies of the present invention can be used in characterizing the IGFBP-3 content of healthy and diseased tissues, through techniques such as ELISAs and Western blotting. This may provide a screen for the presence or absence of cardiomyopathy or as a predictor of heart disease.

The use of antibodies of the present invention, in an ELISA assay is contemplated. For example, anti-IGFBP-3 antibodies are immobilized onto a selected surface, preferably a surface exhibiting a protein affinity such as the wells of a polystyrene microtiter plate. After washing to remove incompletely adsorbed material, it is desirable to bind or coat the assay plate wells with a non-specific protein that is known to be antigenically neutral with regard to the test antisera such as bovine serum albumin. (BSA), casein or solutions of powdered milk. This allows for blocking of non-specific adsorption sites on the immobilizing surface and thus reduces the background caused by non-specific binding of antigen onto the surface.

After binding of antibody to the well, coating with a non-reactive material to reduce background, and washing to remove unbound material, the immobilizing surface is contacted with the sample to be tested in a manner conducive to immune complex (antigen/antibody) formation.

Following formation of specific immunocomplexes between the test sample and the bound antibody, and subsequent washing, the occurrence and even amount of immunocomplex formation may be determined by subjecting same to a second antibody having specificity for IGFBP-3 that differs the first antibody. Appropriate conditions preferably include diluting the sample with diluents such as BSA, bovine gamma globulin (BGG) and phosphate buffered saline (PBS)/Tween®. These added agents also tend to assist in the reduction of nonspecific background. The layered antisera is then allowed to incubate for from about 2 to about 4 hr, at temperatures preferably on the order of about 25° C. to about 27° C. Following incubation, the antisera-contacted surface is washed so as to remove non-immunocomplexed material. A preferred washing procedure includes washing with a solution such as PBS/Tween®, or borate buffer.

To provide a detecting means, the second antibody will preferably have an associated enzyme that will generate a color development upon incubating with an appropriate chromogenic substrate. Thus, for example, one will desire to contact and incubate the second antibody-bound surface with a urease or peroxidase-conjugated anti-human IgG for a period of time and under conditions which favor the development of immunocomplex formation (e.g., incubation for 2 h at room temperature in a PBS-containing solution such as PBS/Tween®).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline)-6-sulfonic acid (ABTS) and H₂O₂, in the case of peroxidase as the enzyme label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody.

The antibody compositions of the present invention will find great use in immunoblot or Western blot analysis. The antibodies may be used as high-affinity primary reagents for the identification of proteins immobilized onto a solid support matrix, such as nitrocellulose, nylon or combinations thereof In conjunction with immunoprecipitation, followed by gel electrophoresis, these may be used as a single step reagent for use in detecting antigens against which secondary reagents used in the detection of the antigen cause an adverse background. Immunologically-based detection methods for use in conjunction with Western blotting include enzymatically-, radiolabel-, or fluorescently-tagged secondary antibodies against the toxin moiety are considered to be of particular use in this regard.

B. Detecting Nucleic Acids

One embodiment of the instant invention comprises a method for detecting variation in the expression of IGFBP-3. This may comprise determining the level of IGFBP-3 or determining specific alterations in the expressed product.

A suitable biological sample can be any tissue or fluid. Various embodiments include cells of the skin, muscle, facia, brain, prostate, breast, endometrium, lung, head & neck, pancreas, small intestine, blood cells, liver, testes, ovaries, colon, skin, stomach, esophagus, spleen, lymph node, bone marrow or kidney. Other embodiments include fluid samples such as peripheral blood, lymph fluid, ascites, serous fluid, pleural effusion, sputum, cerebrospinal fluid, lacrimal fluid, stool or urine.

Nucleic acid used is isolated from cells contained in the biological sample, according to standard methodologies (Sambrook et al., 1989). The nucleic acid may be genomic DNA or fractionated or whole cell RNA. Where RNA is used, it may be desired to convert the RNA to a complementary DNA. In one embodiment, the RNA is whole cell RNA; in another, it is poly-A RNA. Normally, the nucleic acid is amplified.

Depending on the format, the specific nucleic acid of interest is identified in the sample directly using amplification or with a second, known nucleic acid following amplification. Next, the identified product is detected. In certain applications, the detection may be performed by visual means (e.g., ethidium bromide staining of a gel). Alternatively, the detection may involve indirect identification of the product via chemiluminescence, radioactive scintigraphy of radiolabel or fluorescent label or even via a system using electrical or thermal impulse signals (Affymax Technology; Bellus, 1994).

Various types of defects may be identified by the present methods. Thus, “alterations” should be read as including deletions, insertions, point mutations and duplications. Point mutations result in stop codons, frameshift mutations or amino acid substitutions. Somatic mutations are those occurring in non-germline tissues. Germ-line tissue can occur in any tissue and are inherited. Mutations in and outside the coding region also may affect the amount of IGFBP-3 produced, both by altering the transcription of the gene or in destabilizing or otherwise altering the processing of either the transcript (mRNA) or protein.

It is contemplated that other mutations in the IGFBP-3 genes may be identified in accordance with the present invention. A variety of different assays are contemplated in this regard, including but not limited to, fluorescent in situ hybridization (FISH), direct DNA sequencing, PFGE analysis, Southern or Northern blotting, single-stranded conformation analysis (SSCA), RNAse protection assay, allele-specific oligonucleotide (ASO), dot blot analysis, denaturing gradient: gel electrophoresis, RFLP and PCR™-SSCP.

(i) Primers and Probes

The term primer, as defined herein, is meant to encompass any nucleic acid that is capable of priming the synthesis of a nascent nucleic acid in a template-dependent process. Typically, primers are oligonucleotides from ten to twenty base pairs in length, but longer sequences can be employed. Primers may be provided in double-stranded or single-stranded form, although the single-stranded form is preferred. Probes are defined differently, although they may act as primers. Probes, while perhaps capable of priming, are designed to binding to the target DNA or RNA and need not be used in an amplification process.

In preferred embodiments, the probes or primers are labeled with radioactive species (³²P, ¹⁴C, ³⁵S, ³H, or other label), with a fluorophore (rhodamine, fluorescein) or a chemillumiscent (luciferase).

(ii) Template Dependent Amplification Methods

A number of template dependent processes are available to amplify the marker sequences present in a given template sample. One of the best known amplification methods is the polymerase chain reaction (referred to as PCR™) which is described in detail in U.S. Pat. Nos. 4,683,195, 4,683,202 and 4,800,159, and in Innis et al., 1990, each of which is incorporated herein by reference in its entirety.

Briefly, in PCR™, two primer sequences are prepared that are complementary to regions on opposite complementary strands of the marker sequence. An excess of deoxynucleoside triphosphates are added to a reaction mixture along with a DNA polymerase, e.g., Taq polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

A reverse transcriptase PCR™ amplification procedure may be performed in order to quantify the amount of mRNA amplified. Methods of reverse transcribing RNA into cDNA are well known and described in Sambrook et al., 1989. Alternative methods for reverse transcription utilize thermostable, RNA-dependent DNA polymerases. These methods are described in WO 90/07641 filed Dec. 21, 1990. Polymerase chain reaction methodologies are well known in the art.

Another method for amplification is the ligase chain reaction (“LCR”), disclosed in EPO No. 320 308, incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Methods based on ligation of two (or more) oligonucleotides in the presence of nucleic acid having the sequence of the resulting “di-oligonucleotide”, thereby amplifying the di-oligonucleotide, may also be used in the amplification step of the present invention. Wu et al., (1989), incorporated herein by reference in its entirety.

(iii) Southern/Northern Blotting

Blotting techniques are well known to those of skill in the art. Southern blotting involves the use of DNA as a target, whereas Northern blotting involves the use of RNA as a target. Each provide different types of information, although cDNA blotting is analogous, in many aspects, to blotting or RNA species.

Briefly, a probe is used to target a DNA or RNA species that has been immobilized on a suitable matrix, often a filter of nitrocellulose. The different species should be spatially separated to facilitate analysis. This often is accomplished by gel electrophoresis of nucleic acid species followed by “blotting” on to the filter.

Subsequently, the blotted target is incubated with a probe (usually labeled) under conditions that promote denaturation and rehybridization. Because the probe is designed to base pair with the target, the probe will binding a portion of the target sequence under renaturing conditions. Unbound probe is then removed, and detection is accomplished as described above.

(iv) Separation Methods

It normally is desirable, at one stage or another, to separate the amplification product from the template and the excess primer for the purpose of determining whether specific amplification has occurred. In one embodiment, amplification products are separated by agarose, agarose-acrylamide or polyacrylamide gel electrophoresis using standard methods. See Sambrook et al., 1989.

Alternatively, chromatographic techniques may be employed to effect separation. There are many kinds of chromatography which may be used in the present invention: adsorption, partition, ion-exchange and molecular sieve, and many specialized techniques for using them including column, paper, thin-layer and gas chromatography (Freifelder, 1982).

(v) Detection Methods

Products may be visualized in order to confirm amplification of the marker sequences. One typical visualization method involves staining of a gel with ethidium bromide and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly. Following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, and the other member of the binding pair carries a detectable moiety.

In one embodiment, detection is by a labeled probe. The techniques involved are well known to those of skill in the art and can be found in many standard books on molecular protocols. See Sambrook et al., 1989. For example, chromophore or radiolabel probes or primers identify the target during or following amplification.

One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

In addition, the amplification products described above may be subjected to sequence analysis to identify specific kinds of variations using standard sequence analysis techniques. Within certain methods, exhaustive analysis of genes is carried out by sequence analysis using primer sets designed for optimal sequencing (Pignon et al, 1994). The present invention provides methods by which any or all of these types of analyses may be used. Using the sequences disclosed herein, oligonucleotide primers may be designed to permit the amplification of sequences throughout the IGFBP-3 genes that may then be analyzed by direct sequencing.

(vi) Kit Components

All the essential materials and reagents required for detecting and/or sequencing IGFBP-3 and variants thereof may be assembled together in a kit. This generally will comprise preselected primers and probes. Also included may be enzymes suitable for amplifying nucleic acids including various polymerases (RT, Taq, Sequenase™ etc.), deoxynucleotides and buffers to provide the necessary reaction mixture for amplification. Such kits also generally will comprise, in suitable means, distinct containers for each individual reagent and enzyme as well as for each primer or probe.

C. Promoter Methylation

In another embodiment, the present invention provides methods for examining the IGFBP-3 promoter methylation state. By examining promoter methylation, one can ascertain the activity level of the promoter, and thereby determine expression of the IGFBP-3 protein. A number of different methods are available determining promoter methylation.

(i) Sodium Bisulfite Genomic Sequencing

Bisulfite treatment of single-stranded DNA converts unmethylated cytosines to uracil but does not affect methylated cytosines. Uracil is recognized as thymine by Taq polymerase and, hence, the product of the PCR™ will contain cytosines only at positions where 5-methylcytosines occurred in the starting template DNA.

Bisulfite treatment of genomic DNA requires a relatively large amount of fresh genomic DNA and multiple steps. Briefly the genomic DNA is usually treated as described by Zeschnigk et al. (1999):

An 8-μL aliquot of 3 M NaOH was added to a 4-μg sample of DNA (in 70 μL of water). The solution was incubated for 15 min at 37° C., denatured at 95° C. for 3 min, and immediately cooled on ice.

The denatured DNA solution was mixed with 1 mL of bisulfite reagent (freshly prepared by dissolving 8.1 g of sodium bisulfite into 15 mL of water, adding 1 mL of 40 mM hydroquinone, and adjusting the pH to 5.0 with 3 M NaOH), overlaid with mineral oil, and incubated in the dark for 16 h at 55° C.

The DNA was recovered by adsorbing to 5 μL of glassmilk (GeneClean III Kit; Bio 101, Inc., Vista, Calif.) and eluting with 100 μL of water. For desulfonation, 11 μL of 3 M NaOH was added, and the samples were incubated for 15 min at 37° C. and neutralized by adding 110 μL of 6 M ammonium acetate (pH 7.0).

The DNA was precipitated with ethanol, washed in 70% ethanol, dried, and resuspended in 20 μL of water. The concentration of the bisulfite-treated DNA was estimated with DNA DipSticks™ (Invitrogen Corp., San Diego, Calif.).

The region containing target promoter was amplified from the bisulfite-modified DNA with two rounds of PCR™ by use of nested primers specific to the bisulfite-modified sequence of this region.

The final PCR™ products were sequenced by use of the various automatic sequencers or manual sequencing.

(ii) SSCP After Sodium Bisulfite Treatment

After Sodium Bisulfite Treatment, the region of interest can be amplified with primers that does not contain CpG islands. The differences between conformation of methylated and unmethylated gemomic induce differences in migrations in PAGE gel (Brown et al., 2001).

(iii) Restriction-Enzyme Related Polymerase Chain Reaction (RE-PCR)

The restriction-enzyme related polymerase chain reaction (RE-PCR) is another commonly used method for analysis of promoter methylation status. Suppose the target area of promoter contains consensus sequence of methylation sensitive restriction enzyme cutting site, the methylation of this site would be resistant to the restriction enzyme treatment whereas unmethylation of this site would be vulnerable to the enzyme. This method requires that the target area must contain methylation-sensitive enzyme cutting sites and needs some amount of fresh tissues (Tannapfel et al., 2001).

(iv) Methylation-Specific PCR (MSP) Assay

This assay takes advantage of DNA sequence differences between methylated and unmethylated alleles after bisulfite modification. Reacting DNA with sodium bisulfite converts all unmethylated cytosines to uracil, which is recognized as thymine by Taq polymerase, but does not affect methylated cytosines. Amplification with primers specific for methylated or unmethylated DNA discriminates between methylated and unmethylated DNA. It is a simple and fast way of surveying multiple samples to detect methylation of cytosines in the region of interest. With skillful designing of primers, the DNAs obtained from most sources of samples can be investigated including those obtained from microdissected samples.

VIII. Screening for Modulators or IGFBP-3 Activity

The present invention also contemplates the screening of compounds for various abilities to interact and/or affect IGFBP-3 expression or function. Particular compounds will be those useful in promoting the actions of IGFBP-3 in inhibiting tumor formation, growth or metastasis. In the screening assays of the present invention, the candidate substance may first be screened for basic biochemical activity—e.g., binding to IGFBP-3, increased anti-tumor activity, etc.—and then tested for its ability to modulate activity or expression, at the cellular, tissue or whole animal level.

A. Assay Formats

The present invention provides methods of screening for modulators of IGFBP-3. In one embodiment, the present invention is directed to a method of:

(i) providing a IGFBP-3 polypeptide;

(ii) contacting the IGFBP-3 polypeptide with the candidate substance; and

(iii) determining the binding of the candidate substance to the IGFBP-3 polypeptide.

In yet another embodiment, the assay looks not at binding, but at IGFBP-3 expression. Such methods would comprise, for example:

(i) providing a cell that expresses IGFBP-3 polypeptide;

(ii) contacting the cell with the candidate substance; and

(iii) determining the effect of the candidate substance on expression of IGFBP-3.

In still yet other embodiments, one would look at the effect of a candidate substance on the activity of IGFBP-3.

B. Inhibitors and Activators

An inhibitor according to the present invention may be one which exerts an inhibitory effect on the expression or function/activity of IGFBP-3. By the same token, an activator according to the present invention may be one which exerts a stimulatory effect on the expression or function/activity of IGFBP-3.

C. Candidate Substances

As used herein, the term “candidate substance” refers to any molecule that may potentially modulate IGFBP-3 expression or function. The candidate substance may be a protein or fragment thereof, a small molecule inhibitor, or even a nucleic acid molecule. It may prove to be the case that the most useful pharmacological compounds will be compounds that are structurally related to compounds which interact naturally with IGFBP-3. Creating and examining the action of such molecules is known as “rational drug design,” and include making predictions relating to the structure of target molecules.

The goal of rational drug design is to produce structural analogs of biologically active polypeptides or target compounds. By creating such analogs, it is possible to fashion drugs which are more active or stable than the natural molecules, which have different susceptibility to alteration or which may affect the function of various other molecules. In one approach, one would generate a three-dimensional structure for a molecule like a IGFBP-3, and then design a molecule for its abilityt to interact with IGFBP-3. Alternatively, one could design a partially functional fragment of a IGFBP-3 (binding but no activity), thereby creating a competitive inhibitor. This could be accomplished by x-ray crystallography, computer modeling or by a combination of both approaches.

It also is possible to use antibodies to ascertain the structure of a target compound or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds or may be found as active combinations of known compounds which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be polypeptide, polynucleotide, small molecule inhibitors or any other compounds that may be designed through rational drug design starting from known inhibitors of hypertrophic response.

Other suitable inhibitors include antisense molecules, ribozymes, and antibodies (including single chain antibodies).

It will, of course, be understood that all the screening methods of the present invention are useful in themselves notwithstanding the fact that effective candidates may not be found. The invention provides methods for screening for such candidates, not solely methods of finding them.

D. In Vitro Assays

A quick, inexpensive and easy assay to run is a binding assay. Binding of a molecule to a target may, in and of itself, be inhibitory, due to steric, allosteric or charge-charge interactions. This can be performed in solution or on a solid phase and can be utilized as a first round screen to rapidly eliminate certain compounds before moving into more sophisticated screening assays. In one embodiment of this kind, the screening of compounds that bind to a IGFBP-3 molecule or fragment thereof is provided.

The target may be either free in solution, fixed to a support, expressed in or on the surface of a cell. Either the target or the compound may be labeled, thereby permitting determining of binding. In another embodiment, the assay may measure the inhibition of binding of a target to a natural or artificial substrate or binding partner (such as a IGFBP-3). Competitive binding assays can be performed in which one of the agents (IGFBP-3 for example) is labeled. Usually, the target will be the labeled species, decreasing the chance that the labeling will interfere with the binding moiety's function. One may measure the amount of free label versus bound label to determine binding or inhibition of binding.

A technique for high throughput screening of compounds is described in WO 84/03564. Large numbers of small peptide test compounds are synthesized on a solid substrate, such as plastic pins or some other surface. The peptide test compounds are reacted with, for example, a IGFBP-3 and washed. Bound polypeptide is detected by various methods.

Purified target, such as a IGFBP-3, can be coated directly onto plates for use in the aforementioned drug screening techniques. However, non-neutralizing antibodies to the polypeptide can be used to immobilize the polypeptide to a solid phase.

E. In Cyto Assays

Various cell lines that express IGFBP-3 can be utilized for screening of candidate substances. For example, cells containing a IGFBP-3 with engineered indicators can be used to study various functional attributes of candidate compounds. In such assays, the compound would be formulated appropriately, given its biochemical nature, and contacted with a target cell.

Depending on the assay, culture may be required. As discussed above, the cell may then be examined by virtue of a number of different physiologic assays (growth, colony formation, etc.). Alternatively, molecular analysis may be performed in which the function of a IGFBP-3 and related pathways may be explored. This involves assays such as those for protein expression, enzyme function, substrate utilization, mRNA expression (including differential display of whole cell or polyA RNA) and others.

F. In Vivo Assays

The present invention particularly contemplates the use of various animal models. Transgenic animals may be created with constructs that permit IGFBP-3 expression and activity to be controlled and monitored. The generation of these animals has been described elsewhere in this document.

Treatment of these animals with test compounds will involve the administration of the compound, in an appropriate form, to the animal. Administration will be by any route the could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply.

G. Production of Modulators

In an extension of any of the previously described screening assays, the present invention also provide for method of producing modulators. The methods comprising any of the preceding screening steps followed by an additional step of “producing the candidate substance identified as a modulator of” the screened activity.

### IX. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Study Population

Tissue specimens were obtained at surgery from a total of 74 patients whose diagnosis revealed NSCLC and who had undergone curative surgical removal of a primary lesion at The University of Texas M. D. Anderson Cancer Center (UT-MDACC) from October 1975 through April 1993. Pathological evaluation established the histological classification and staging in all of the patients. None of the patients had either radiotherapy or chemotherapy before or after surgery until the disease recurred. Patients' ages ranged from 37.8 to 82.7 years, with a mean age of 63.2±9.48 years, which is similar to the age distribution in the large database of patients with stage I NSCLC from (UT-MDACC; data not shown). Fifty-four (73%) of the patients were men and 20 (27%) were women. All of the clinical and pathological information and follow-up data were based on reports from the tumor registry service at UT-MDACC. The study was reviewed and approved by the institution's Surveillance Committee to allow the tissue blocks and other pertinent information to be obtain from the patients' files.

Immunohistochemical Staining for IGFBP-3

Paraffin-embedded, 4-μm-thick tissue sections from 74 primary NSCLC samples were stained for the IGFBP-3 protein using a rabbit polyclonal antibody against human IGFBP-3 (Diagnostic Systems Laboratories, Inc., Webster, Tex.). All of the sections were deparaffinized in a series of xylene baths and then rehydrated using a graded alcohol series. The sections were then immersed in methanol containing 0.3% hydrogen peroxidase for 20 mm to block endogenous peroxidase activity and then incubated in 2.5% blocking serum to reduce nonspecific binding. Sections were incubated overnight at 4° C. with primary anti-IGFBP-3 antibody (1:100). The sections were then processed using standard avidin-biotin immunohistochemical techniques according to the manufacturer's recommendations (Vector Laboratories, Burlingame, Calif.). Diaminobenzidine was used as a chromogen, and commercial hematoxylin was used for counterstaining. Three human NSCLC cell lines, H1944, H460, and A549, which have intrinsic IGFBP-3 expression, were stained at the same time to serve as positive controls. Adjacent normal appearing bronchial epithelium within each tissue section served as an internal reference. Representative areas of each tissue section were selected, and cells were counted in at least four fields (at ×200). IGFBP-3 labeling index was defined as the percentage of tumor cells displaying membranous, cytoplasmic, or nuclear immunoreactivity; and it was calculated by counting the number of IGFBP-3-stained tumor cells among more than 1000 tumor cells from representative areas of each tissue section. In this study, a 5% labeling index was used as a cutoff point. On the basis of the results of the immunohistochemical staining, tissue sections showing less than 5% of positive staining were considered as down-regulation of IGFBP-3. All of the slides were evaluated and scored independently. The pathologists were blinded to the clinical information of the subjects.

Statistical Analysis

In univariate analysis, independent sample t tests and x² tests were used for continuous and categorical variables, respectively. The Kaplan-Meier estimator was used to compute survival probability as a function of time. The log-rank test was used to compare patients' survival time between groups. Overall, disease-specific, event-free, and disease free survival times were analyzed. Cox regression was used to model the risk of the loss of expression on survival time, with adjustment for clinical and histopathological parameters (age, sex, tumor histology subgroup, grade of differentiation, and smoking status). All of the statistical tests were two-sided. P<0.05 was considered to be statistically significant.

### Example 2

### Results

Expression of IGFBP-3 in Histologically Normal Lung Tissues and NSCLC

The staining for IGFBP-3 was prominent in the cytoplasm of histologically normal bronchial epithelial cell layers. The intensity of the staining was moderate to strong, and the distribution was homogeneous. The expression of IGFBP-3 was also noted in the epithelium of small airways. The staining pattern of normal bronchial epithelium was consistent and was used as a reference. In addition, frequent nuclear staining was also observed in the basal, parabasal, and ciliated cells. The human NSCLC cell lines, H1944, H460, and A549 NSCLC cells, which exhibited strong IGFBP-3 expression by Western blot analysis, were used as a positive control, and H226Br cells, which showed no IGFBP-3 expression, were used as a negative control. Normal cells showed a homogeneous staining pattern for IGFBP-3, but in the majority of tumor tissue, a heterogeneous pattern of negative staining, scattered positive staining, and positive staining was seen. In contrast to the previous reports of nuclear staining of the IGFBP-3 in NSCLC cell lines (Jaques et al., 1997), it was observed that IGFBP-3 expression was localized mainly in the cytoplasm and was not detected in the nuclei of tumor cells. In addition, histologically well-differentiated tumors showed more frequent and intense IGFBP-3 staining, although it was not statistically significant (P=0.186). Five cases showed typical membranous staining that was not related to histological subtype or grade; two cases were adenocarcinoma, two cases were squamous cell carcinoma, and the other was diagnosed as a large cell carcinoma.

Clinicopathological Parameters Associated With Loss of IGFBP-3 Expression

To date, there has been no available labeling index for the staining of IGFBP-3 in NSCLC; therefore, the inventor applied a 5% labeling index as a cutoff point for the down-regulation of IGFBP-3. On the basis of this criterion, 42 (56.8%) of the 74 stage I NSCLC specimens showed a loss of IGFBP-3 expression The associations between the IGFBP-3 expression status and the clinicopathological parameters were summarized. The IGFBP-3 expression status did not differ significantly with respect to age, gender, smoking status, pack-years, or histological grade of differentiation. There were no differences in the frequency of loss of expression between adenocarcinoma and squamous carcinoma, but a loss of IGFBP-3 expression was, however, frequent in large cell and unspecified carcinomas. The smoking status of 71 of 74 patients was known; 69 patients had been smokers, and 67 were current smokers at the time of diagnosis. The mean number of packyears for these 67 people was 62.7±39.41. There was also no difference in the distribution of smoking status or pack-years between groups that showed down-regulation of IGFBP-3 and those that did not show down-regulation. Three patients had a history of exposure to asbestos, but the associations between IGFBP-3 expression and this parameter are not provided because of the small sample size.

Down-Regulation of IGFBP-3 Expression in NSCLC Related to Patients' Prognosis

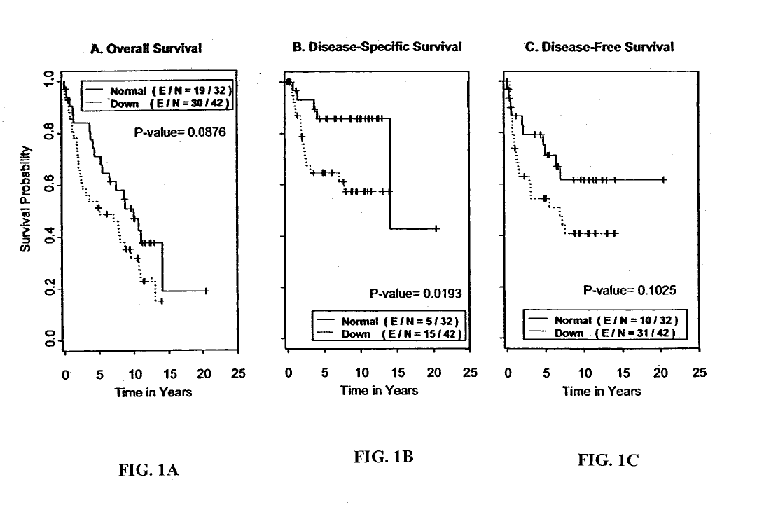

The relationship between IGFBP-3 expression and patients' clinical outcomes was analyzed. The probability of 5-year overall survival in this study population was 59.8% (95% confidence interval, 49.5-72.3), which is similar to the probabilities reported in a previous study with a large number of cases from UT-MDACC (Mountain et al., 1997). Of the 74 patients, 49 patients died, and 25 patients were still alive at the time of the last follow-up report. Of the 49 patients who died, 20 died of lung cancer, and 29 patients died of other causes. The median follow-up duration among the patients who remained alive was 10.5 years. Thirty (71.4%) of the 42 patients whose tumors showed loss of IGFBP-3 expression were dead, whereas 19 (59.4%) of the 32 patients whose tumors showed IGFBP-3 expression were dead during the follow-up time. However, the difference did not reach statistical significance (P=0.0876 by log-rank test; FIG. 1A). Of the 42 patients whose tumors lost expression of IGFBP-3, 15 (35.7%) patients died of cancer or a cancer-related cause; only 5 (15.6%) of the 32 patients whose tumors showed IGFBP-3 expression died of cancer or a related cause. Patients who have tumors with low IGFBP-3 expression showed significantly shorter disease-specific survival (P=0.0193 by log-rank test; FIG. 1B). The 5-year disease-free survival probability for patients whose tumors showed loss of IGFBP-3 expression was 54.4% as compared with 71.4% in patients whose tumors showed IGFBP-3 expression (P=0.1025 by log-rank test; FIG. 1C). In a multivariate analysis using IGFBP-3 expression and other clinicopathological parameters, IGFBP-3 remained an independent prognostic factor for disease-specific survival time (P=0.0124).

### Example 3

### Materials and Methods

Animals, Cells, and Materials

Four-week-old female nude mice were purchased from Harlan-Sprague-Dawley (Indianapolis, Ind.). Normal human bronchial epithelial (NHBE) cells were grown from the bronchial epithelium as previously described (30). NSCLC cell lines (H1299, H661, H596, A549, H460, H358, H322, H226Br, H226B, Calu6, Calu1, ChagoK, and SK-MES-1) were routinely maintained in RPMI 1640 medium supplemented with 10% fetal calf serum (FCS) (GIBCO-BRL, Gaithersburg, Md.) in a humidified environment with 5% CO₂. 293 cells were maintained in DMEM containing 10% FCS (GIBCO-BRL). Total IGF-I and -II were purchased from R&D Systems, Inc. (Minneapolis, Minn.). Fluorolink Cy3-labeled secondary antibody was purchased from Amersham Corp. (Arlington Heights, Ill.) and rabbit polyclonal antibodies against human anti-pAKT (Ser473), Akt, and pGSK-3β (Ser9) were purchased from New England Biolabs (Beverly, Mass.). Rabbit polyclonal anti-GSK-β antibody (BD Transduction Laboratories, Lexington, Ky.), rabbit polyclonal anti-Bax and anti-caspase-3 antibodies (Pharmingen, San Diego, Calif.), rabbit polyclonal anti-Bcl-2 and rabbit polyclonal anti-PARP antibody (VIC 5) (Roche Molecular Biochemicals, Indianapolis, Ind.), rabibit polyclonal anti-IGFBP-3 (Santa Cruz Biotechnology, Inc., Santa Cruz, Calif.), goat antibodies against ERK-1, ERK-2, and β-Actin (Santa Cruz Biotechnology, Inc.), and goat polyclonal anti-human IGFBP-3 antibody (Diagnostic Systems Laboratories, Webster, Tex.) were used for Western blot analysis or immunofluorescence confocal microscopy. The expression construct pCMV6.MyrAktHA contained a myristoylation sequence fused in-frame to the c-Akt coding sequence (MyrAkt). The expression vector pCMV.MCL⊕HA-MAPKK (MEK1/R4F) was also used.

Generation of Ad5CMV-BP3

A full-length human IGFBP-3 cDNA was inserted into the 5′ end of the bovine growth hormone polyadenylation signal at EcoRV of the pAd-shuttle vector. The IGFBP-3-containing shuttle vector was digested with BstB1/ClaI, inserted into the pAd-speed vector containing adenoviral DNA, and transfected into 293 cells. 293 cells were maintained in DMEM containing 10% FCS until the onset of the cytopathic effect. The presence of IGFBP-3 was confirmed by dideoxy-DNA sequencing and Western blot analysis. Viral titers were determined by plaque assays and spectrophotometric analysis

Western and Western Ligand Blot Analyses

Conditioned media were collected from H1299 cells after adenoviral infection. Western blot and Western ligand blot analyses were performed using 30 μg of whole cell lysate or 30 μl of conditioned media as previously described (Sueoka et al., 2000).

Measurements of Cell Growth

To measure the effects of IGF-I and IGF-II on proliferation of NSCLC cells, 2×10³ NSCLC cell lines were incubated in serum-free medium containing 0.01-500 ng/ml IGF-I or IGF-II for 3 days. To measure the effects of Ad5CMV-BP-3 on proliferation of NHBE and NSCLC cell lines, these cells were seeded at 1×10^{-2×10}³ cells/well in 96-well plates. After 1 day, cells were untreated or infected with 1×10³, 5×10³, or 1×10⁴ particles/cell of Ad5CMV-BP3 or Ad5CMV (parental virus) as a viral control. Infection was allowed to occur for 2 h in the absence of serum, and infected cells were grown in medium containing 100 ng/ml or 250 ng/ml IGF-I. After 3 days of incubation, the growth of infected cells was measured by the 3-(4,5-dimethylthiazol-2-yl)-2,5-diphenyltetrazolium bromide (MTT) assay as previously described (Sueoka et al., 2000).

Growth in Soft Agar

The ability of NSCLC cells infected with Ad5CMV-BP3 to grow in anchorage-independence was assessed in soft agar. Briefly, 1×10³H1299 cells were transduced with 1×10³ or 1×10⁴ particles/cell of Ad5CMV or Ad5CMV-BP3 for 1 day. They were then suspended in 1 ml of RPMI 1640 medium containing 10% FCS and 0.2% agarose and were plated in 12-mm tissue culture plates with 500 μl of RPMI 1640 medium containing 10% FCS and a 1% agarose underlay. After 7 days, cultures were resuspended in 0.2 ml RPMI 1640 medium containing the same viruses at the same doses. Anchorage-independent growth was allowed to occur for 2 weeks, and colonies >125 μm in diameter were counted.

Inhibition of Tumor Growth In Vivo

The effect of Ad5CMV-BP3 on established subcutaneous tumor nodules was determined in athymic nude mice in a defined pathogen-free environment. Briefly, mice were irradiated with 350 rad (¹³⁷CS source), and H1299 cells in 100μof complete medium were subcutaneously injected into the mice at a single dorsal site. After the tumor volume reached approximately 75 mm³, 1×10¹⁰ viral particles of Ad5CMV-BP3 or Ad5CMV in 100 μl of 1×PBS, or 100 μl of 1×PBS alone as a control, was intratumorally injected. Tumor size and volume were measured every day for 17 days after injection. Mice showing necrotic tumors or tumors >1.5 cm in diameter were euthanized. Results were expressed as the mean (±standard deviation) tumor volume (calculated from 5 mice) relative to the tumor volume at the time of adenovirus injection (day 0).

Apoptosis Analysis

Apoptosis was measured using the APO-BRDU staining kit (Phoenix Flow Systems, San Diego, Calif.) as previously described (Sueoka et al., 2000). Briefly, H1299 cells were untreated or infected with Ad5CMV or Ad5CMV-BP3, and then allowed to grow in serum-free medium or medium containing 10% serum or 100 ng/ml IGF-I for 3 days. Floating and adherent cells were analyzed using a FACScan flow cytometer (Becton Dickinson, San Jose, Calif.) to determine the percentage of apoptotic cells. The percentage of dead cells was determined by FACS analysis of propidium iodide-stained nuclei. Apoptosis was also determined by detecting of nucleosomal DNA fragmentation, which was measured using the TACS apoptotic DNA laddering kit (Trevigen, Inc., Gaithersburg, Md.) according to the manufacturer's protocol. Briefly, DNA was isolated from untreated or virus-infected H1299 cells by incubating the cells in lysis buffer. DNA samples were then subjected to electrophoresis on a 1.5% agarose gel and visualized by ethidium bromide staining. To determine whether AdSCMV-BP3-induced apoptosis was mediated through the inhibition of the PI3K/Akt/PKB and MAPK pathways, 2×10⁵H1299 cells were seeded onto 6-well plates and transiently transfected with 2 μg of an expression construct containing constitutively active Akt (MyrAkt) or constitutively active mitogen-activated protein kinase kinase (MAPKK, MEK1/R4F) using the FuGENE 6 transfection reagent (Roche Molecular Biochemicals) according to the manufacturer's protocol. After 4 h of transfection, H1299 cells were infected for 2 h with 1×10⁴ particles/cell of Ad5CMV-BP3 or Ad5CMV as a control. Cells were changed to fresh RPMI 1640 medium containing 10% FCS and grown for 3 days. Apoptosis was measured using the APO-BRDU staining kit as described above.

Immunofluorescence Confocal Microscopy

To determine whether Ad5CMV-BP3 induced IGFBP-3 expression and apoptosis in tumor nodules in nude mice, 1×10¹⁰ viral particles of Ad5CMV-BP3 or Ad5CMV were intratumorally injected as described above, and tumor tissues were collected from the mice 3 days later. Tissues were fixed with 10% formaldehyde and embedded in paraffin. Then, 5-μm-thick tumor tissue sections were analyzed for IGFBP-3 expression and apoptosis-induced DNA fragmentation. Briefly, sections were deparaffinized through a series of xylene baths, and rehydrated through graded ethanol baths. Next, the sections were treated with 2.5% blocking serum to reduce nonspecific binding and were incubated with primary anti-human IGFBP-3 rabbit polyclonal antibody, 1:100 dilution, and followed by incubation with Fluorolink Cy3-labeled secondary antibody. Paraffin-embedded tissue sections were analyzed for DNA fragmentation by TdT (terminal deoxynucelotidyl transferase)-mediated dUTP-biotin nick-end labeling (TUNEL) assay according to the manufacturer's protocol (Roche Molecular Biochemicals). Samples were analyzed using an inverted confocal microscope (Zeiss Inc., Jena, Germany) operated by KS400 software (Zeiss Inc.).

Immune Complex Kinase Assay

Immune complex kinase assays were performed as previously described (Lee et al., 1998). Briefly, untreated or virus-infected H1299 cells were grown for 2 days in RPMI 1640 medium containing 10% serum. After being washed with 1×PBS to eliminate residual serum, cells were serum starved for 1 day, activated by treatment with 50 ng/ml IGF-I for 15 min, and harvested using lysis buffer. Next, 100 μg of cell extracts was immunoprecipitated with a mixture of antibodies against p44 MAPK (ERK1)/p42 and MAPK (ERK2) and with protein A-G agarose beads. After the beads were washed, an immune complex kinase assay was performed using myelin basic protein (MBP) as a substrate.

### Example 4

### Results

IGFBP-3 Expression in NSCLC Cells Transduced by Ad5CMV-BP3

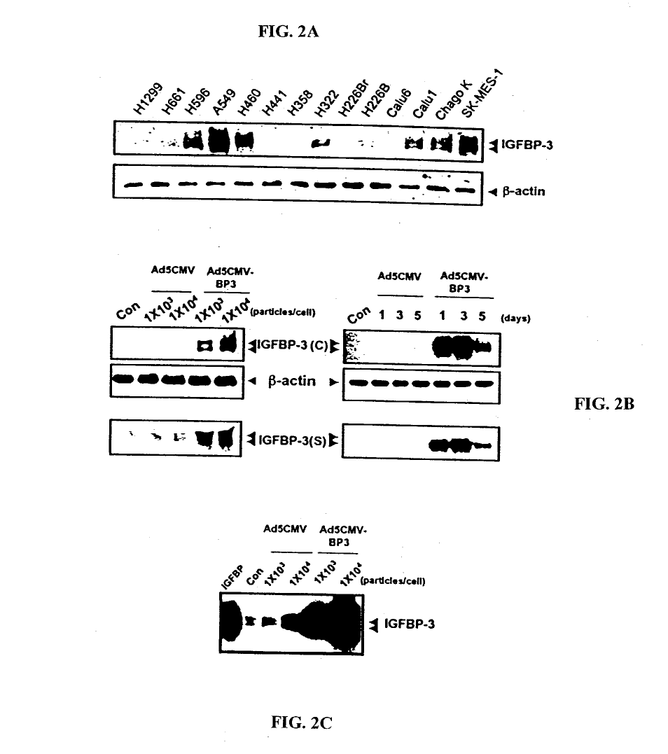

Induction of IGFBP-3 expression by Ad5CMV-BP3 was analyzed by Western blot analysis on H1299, H661, H441, H358, H226Br, H226B, and Calu6 NSCLC cell lines, which showed very low or no IGFBP-3 protein expression (FIG. 2A). Representative results showing viral dose-dependent increase in IGFBP-3 protein level (42-kDa and 44-kDa forms) in H1299 cells (C) or secreted into medium (S) are shown in FIG. 2B. IGFBP-3 expression was not detectable in the cells incubated with medium alone or with AdSCMV. The expression of IGFBP-3 peaked at day 3, and decreased at day 5 (FIG. 2B). These results are consistent with those of previous reports showing maximal adenovirus-mediated gene expression at day 3 and rapid decrease after day 5 (Zhang et al., 1994). The expression pattern in other NSCLC cell lines infected with Ad5CMV-BP3 was consistent with that in H1299 cells. The Western ligand blot analysis using conditioned media from H1299 cells infected with Ad5CMV-BP3 or with Ad5CMV indicated that IGFBP-3 secreted from Ad5CMV-BP3-infected H1299 cells bound strongly to IGF-I (FIG. 2C) and IGF-II.

Effect of IGFBP-3 on IGF-induced NSCLC Cell Growth

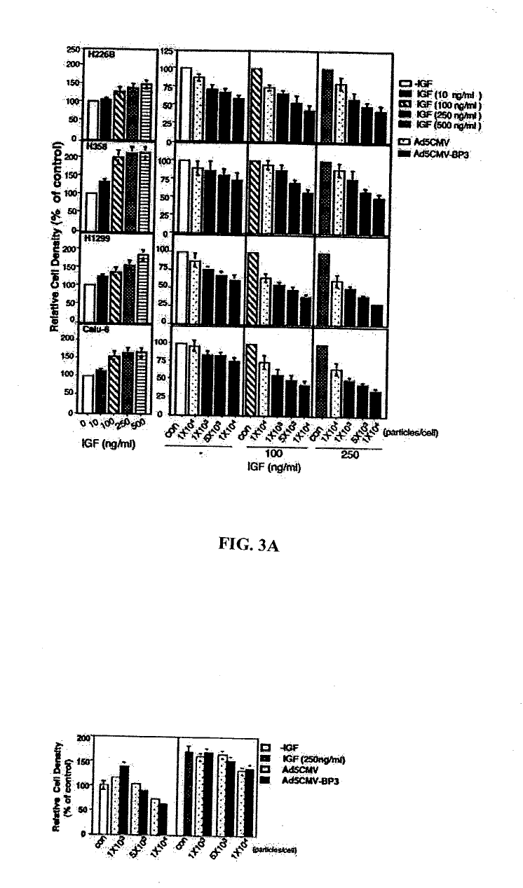

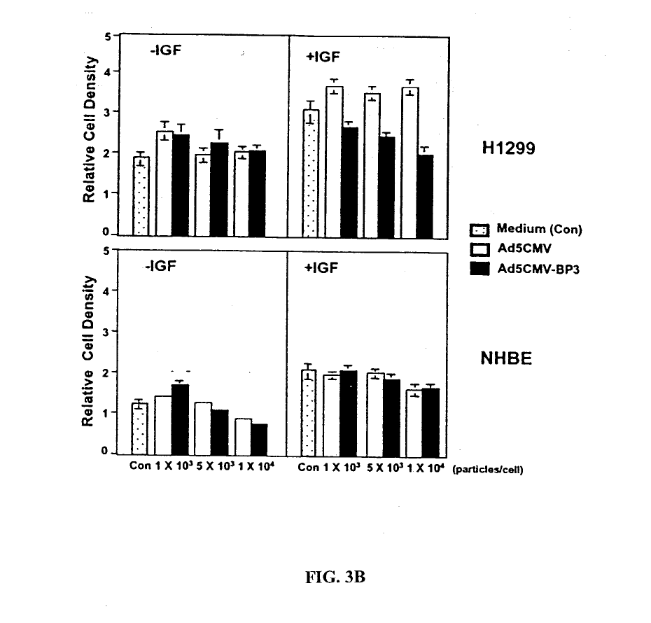

The effect of IGFBP-3 on IGF-induced NSCLC cell growth was investigated by MTT assay. IGF-I concentrations >10 ng/ml induced the growth of NSCLC cell lines (FIG. 3A), showing the mitogenic effect of IGF-I on NSCLC cells. IGF-II showed a minimal mitogenic effect on NSCLC cell growth. Hence, the inventor investigated the effects of Ad5CMV-BP3 on IGF-I-stimulated NSCLC cell growth. Relative to the control, Ad5CMV-BP3 infection inhibited IGF-I-induced growth of NSCLC cells in a viral dose-dependent manner. In contrast, Ad5CMV-BP3 showed minimal inhibitory effects on NSCLC cell growth in the absence of IGF-I. Importantly, Ad5CMV-BP3 was not detectably cytotoxic to NHBE cells regardless of the stimulation of IGF-I (FIG. 3B).

Growth in Soft Agar

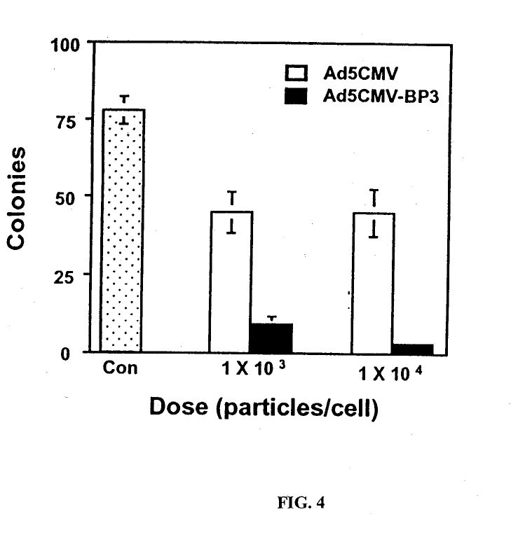

IGF-1R is particularly important in anchorage-independent growth, and previous studies have suggested that its inhibition also suppresses tumorigenicity (Baserga, 1999; Lee et al., 1996a). Therefore, anchorage-independent growth of H1299 cells infected with Ad5CMV-BP3 was assessed by counting colony formation in soft agar. Relative to the effect of controls, Ad5CMV-BP3 infection prominently inhibited colony formation of H1299 cells in soft agar, with a 90% decrease in cells infected with 1×10⁴ particles/cell of AdSCMV-BP3 (FIG. 4). These findings suggested that Ad5CMV-BP3 is capable of suppressing the tumorigenicity of H1299 NSCLC cells.

Inhibition of Tumor Growth In Vivo

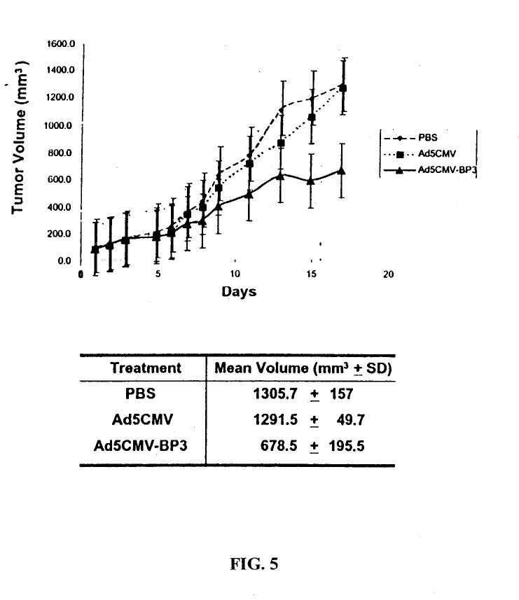

To further investigate the growth regulatory effects of IGFBP-3 in an in vivo setting, the inventor determined the effect of AdSCMV-BP3 on established subcutaneous tumor nodules in athymic nude mice. Once, H11299 xenograft tumors reached a volume of at least 75 mm³, 1×10¹⁰ viral particles of Ad5CMV-BP3 or Ad5CMV in 1×PBS, or 1×PBS alone as a control was intratumorally injected, and tumor size was measured every day for 17 days. Ad5CMV-BP3 injection significantly reduced tumor volume (mean volume, 678.5+195.5 mm³), when compared with tumors injected with Ad5CMV (mean volume, 1,291.5+49.7 mm³) or 1×PBS (mean volume, 1,305+157 mm³). The mean size of Ad5CMV-BP3-injected tumors was reduced by 48% by day 17 (FIG. 5).

Induction of Apoptosis by IGFBP-3 In Vitro and In Vivo

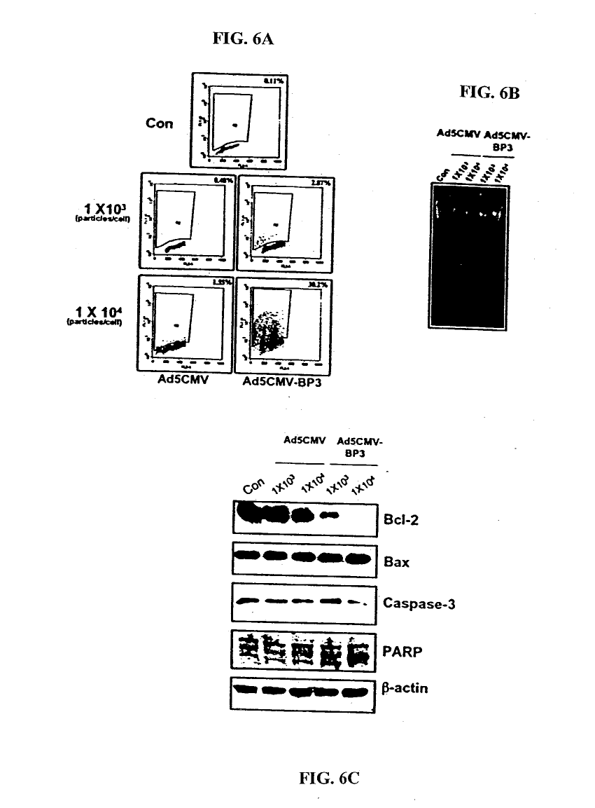

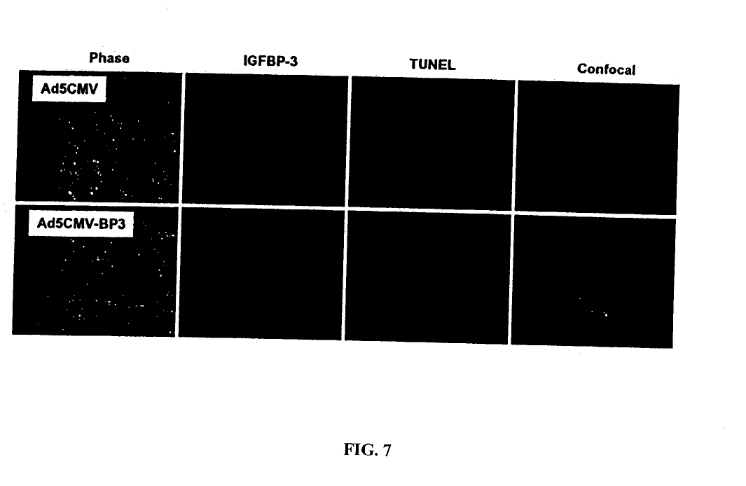

The inventor investigated the mechanism by which IGFBP-3 inhibited NSCLC cell growth. Because IGFBP-3 is a potent inducer of apoptosis (Lin et al., 1999; Butt et al., 2000; Yu et al., 1999; Rajah et al., 1997; Valentinis et al., 1995), evidence for apoptosis following Ad5CMV-BP3 was examined. Indeed, NSCLC cells infected with Ad5CMV-BP3 shrank and detached from their culture dishes. Flow cytometric analysis of H1299 cells revealed that AdSCMV-BP3 infection induced a comparable increase in incorporation of Br-dUTP, with 30.2% of the cells infected with 1×10⁴ particles/cell of Ad5CMV-BP3 were apoptotic (FIG. 6A). DNA fragmentation analysis on H1299 cells infected with 1×10⁴ particles/cell of Ad5CMV-BP3 showed DNA ladders (FIG. 6B). Because the Bcl protein family has an important role in apoptosis, the inventor examined the level of Bcl-2 and Bax in Ad5CMV-BP3-infected H1299 cells using Western blot analysis. Ad5CMV-BP3 inhibited the expression of Bcl-2 in a dose-dependent manner without changing the level of Bax, suggesting a role of IGFBP-3 in modulating the Bax:Bcl-2 ratio (FIG. 6C). Western blot analysis was also performed to determine whether AdSCMV-BP3 induced a loss of the caspase-3 proenzyme (32 kDa) and cleavage of PARP, a substrate of caspase-3 proteolysis. H1299 cells infected with 1×particles/cell of AdSCMV-BP3 for 3 days showed a significant decrease in the 32-kDa caspase-3 proenzyme and in induction of the 89-kDa fragment of PARP cleaved from the 113-kDa form of PARP. These findings indicate that IGFBP-3 is a potent inducer of apoptosis in NSCLC cell lines. To further investigate whether induction of IGFBP-3 caused apoptosis in vivo, immunofluorescence analysis and TUNEL assay were performed on tumors that were removed 3 days after a single administration of adenovirus. Compared to the effect of AdSCMV, Ad5CMV-BP3 injection markedly increased IGFBP-3 and TUNEL staining (FIG. 7), indicating that the expression of IGFBP-3 induced apoptosis in tumor tissues. Only minimal TUNEL staining occurred in the Ad5CMV-injected tumors; probably because of the toxicity of the empty virus. Confocal microscopy revealed that IGFBP-3 expression and DNA fragmentation were colocalized, indicating that the expression of IGFBP-3 induced apoptosis. Some regions showed strong TUNEL staining but no staining for IGFBP-3; this apoptosis may have been spontaneous or induced by IGFBP-3 that was degraded after induction of apoptosis.

Modulation of the PI3K and MAPK Pathways by IGFBP-3 in NSCLC Cells

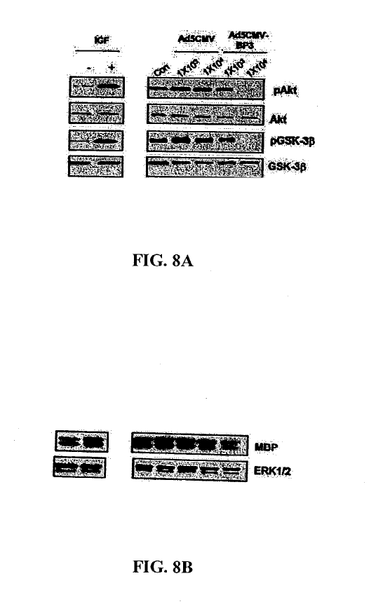

IGFBP-3 has the potential to function as an antagonist of the PI3K and MAPK pathways because these pathways are activated by the IGF-I-induced signaling transduction mechanism (Wang et al., 1998; Lin et al., 1999). To address this possibility, the inventor determined the effect of Ad5CMV-BP3 on the PI3K and MAPK pathways in Ad5CMV-BP3-infected H1299 cells. Activation of the PI3K pathway generally causes selective phosphorylation of a downstream effector, such as Akt at Ser473/Thr308 and GSK-3α/β at Ser9/21(31); therefore, the inventor examined the levels of pAkt (Ser473) and pGSK-3β (Ser9) as surrogates for PI3K activity. According to results of Western blot analysis and an immune complex kinase assay, treatment with 50 ng/ml IGF-I for 15 min increased the levels of pAkt and pGSK-3β and induced MAPK activity in H1299 cells. Ad5CMV-BP3 inhibited IGF-I-induced phosphorylation of Akt (Ser473) and GSK-3β (Ser9) in H1299 cells in a dose-dependent manner, whereas the levels of pAkt (Ser473) and pGSK-3β (Ser9) were not affected by IGF-I in untreated and Ad5CMV-infected H1299 cells (FIG. 8A). The total protein levels of Akt and GSK-3β were not changed after any of these treatments.

According to the immune complex kinase assay, Ad5CMV-BP3 infection also decreased MAPK activity without changing the protein levels of p44 MAPK (ERK1) and p42 MAPK (ERK2) in H1299 cells (FIG. 8B), suggesting that IGFBP-3 suppressed the IGF-I-induced activation of the PI3K/Akt/PKB and MAPK pathways in H1299 cells.

Overexpression of Constitutively Active Akt or MEK1 Protects H1299 Cells from IGFBP-3-Induced Apoptosis

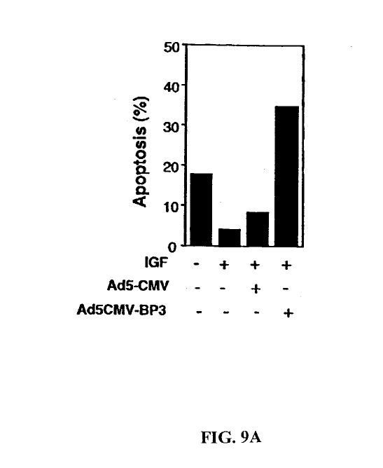

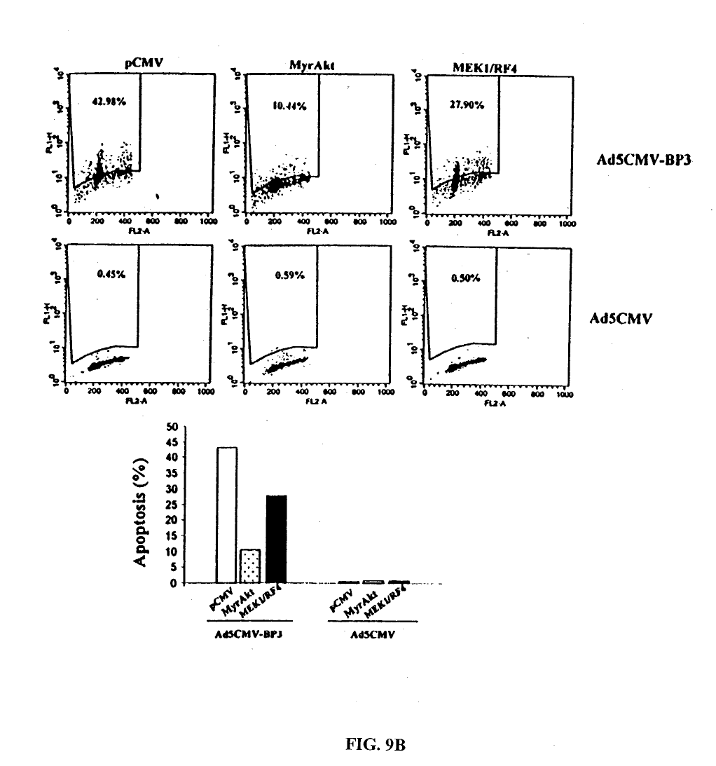

The inventor investigated whether IGFBP-3 inhibits IGF-induced cell survival pathways in NSCLC cells. Evidence of apoptosis was assessed in H1299 cells, which were untreated or infected with Ad5CMV or Ad5-CMV-BP3, and then allowed to grow in the absence or presence of IGF-I. According to flow cytometric analysis, IGF-I treatment rescued H1299 cells from serum depletion-induced apoptosis in H1299 NSCLC cells. However, this rescue was blocked by the overexpression of IGFBP-3 (FIG. 9A), suggesting that IGFBP-3 interferes with survival function of IGF-I. To further explore whether IGFBP-3-induced apoptosis was mediated through the inhibition of IGF-1-induced signaling pathways, the susceptibility to the induction of apoptosis by Ad5CMV-BP3 was assessed in H1299 cells transfected with constitutively active Akt (MyrAkt) or constitutively active MEK1 (R4F) followed by the infection with Ad5CMV-BP3 or Ad5CMV. The equal transfection in each condition was verified by Western blot analysis on MyrAkt and MEK1 (data not shown). According to flow cytometry analysis, 10.4% of MyrAkt-transfected H1299 cells and 27.9% of MEK1-transfected cells showed induction of apoptosis by 1×10⁴ particles/cell of Ad5CMV-BP3, as compared to 43% of induction in pCMV (empty vector)-transfected cells (FIG. 9B), suggesting that the induction of apoptosis by IGFBP-3 in NSCLC cells was due in part to the inhibition of the IGF-induced PI3K/Akt/PKB and MAPK pathways. Taken together, these results suggested a crucial role of IGFBP-3 in PI3K/Akt/PKB and MAPK-mediated cell survival pathways.

### Example 5

### Materials and Methods

Cell Cultures

Human bronchial epithelia (HBE) cells were grown from bronchial epithelium in keratinocyte serum-free medium (Gibco/BRL, Gaithersburg, Md.) as described previously (Lee et al., 1998). For each study, HBE cells from a single patient were used. Human NSCLC cell lines H1299, H661, H596, A549, H460, H441, H358, H322, H1944, H292, H226B, Calu-6, Calu-1, and H157 were purchased from American Type Tissue Collection (Manassas, Va.). The cell lines were cultured in RPMI1640 medium supplemented with 10% fetal calf serum (FCS) (Gibco/BRL).

Northern Blot Analysis

NSCLC cell lines and H1299 cells treated with 0.1, 1, or 5 μM of 5-aza-dC (Sigma, St. Luois, Mo.) for 5 days in RPMI 1640 medium containing 2% FCS were lysed in 4.0 M guanidinium isothiocyanate, and total cellular RNA was extracted as described previously (Kim et al., 1995). 20 μg of RNA was subjected to electrophoresis on a 1% agarose gel containing 2% formaldehyde. After gels were soaked in 50 mM NaOH/1×standard saline citrate (SSC) for 20 min followed by 10×SSC for 20 min, RNA was transferred onto a Zeta-Probe membrane (Bio-Rad Laboratories, Hercules, Calif.) overnight by the capillary transfer method. Membranes were cross-linked and then incubated in the hybridization solution containing [γ-³²P]dCTP-labeled IGFBP-3 or glyceraldehyde-3-phosphate dehydrogenase (GAPDH) cDNA as a control. The probe was labeled using a Prime-It® II Random Primer Kit (Stratagene, La Jolla, Calif.) and harvested with MicroSpin™ S-300 HR Columns (Amersham Phamacia Biotech Inc., Piscataway, N.J.). After overnight incubation, membranes were washed in high stringency condition and exposed to enhanced chemiluminescence (ECL) films for autoradiography.

Clinical Samples

Formalin-fixed, paraffin-embedded tissue blocks of lung cancer were obtained from surgical specimens of a total of 123 patients who were diagnosed with NSCLC and had undergone surgical removal of a primary lesion at UTMDACC from 1975 through 1998. Epithelial cells from bronchial brush samples were collected from 10 healthy volunteers from an ongoing chemoprevention clinical trial and served as negative controls. Tissue sections (4 μm thick) were obtained from each block, stained with hematoxylin-eosin, and reviewed by a pathologist to confirm the diagnosis and the presence or absence of tumor cells in these sections. All information, including clinical, pathological, and follow-up data, was based on reports from the tumor registry service at UTMDACC. The study was reviewed and approved by the institution's Surveillance Committee which allowed tissue blocks and all pertinent information on patients to obtained.

Microdissection and DNA extraction

DNA was extracted from micro-dissected tumor specimens as described previously (Kim et al., 1997; Mao et al., 1996). Briefly, tumor parts in sections from formalin-fixed and paraffin-embedded tissue blocks were dissected under a stereomicroscope. Dissected tissues were digested in 200 μL of digestion buffer containing 50 mL Tris-HCl (pH 8.0), 1% sodium dodecyl sulfate, and proteinase K (0.5 mg/mL) at 42° C. for 36 h. The purification of digested products was performed by phenol/chloroform extraction. DNA was then precipitated by the ethanol precipitation method in the presence of glycogen (Boehringer Mannheim Biochemicals, Indianapolis, Ind.) and recovered in distilled water.

Bisulfite Modification and Methylation-Specific Polymerase Chain Reaction

200 ng of DNA from the micro-dissected tumor samples and NSCLC cell lines was mixed with 1 μg of salmon sperm DNA (Life Technologies, Inc., Gaithersburg, Md.), and submitted for chemical modification as described by Herman et al. (1996). Briefly, DNA was denatured with 2 M NaOH, followed by treatment with 10 mM hydroquinone and 3 M sodium bisulfite (Sigma Chemical Co., St. Louis, Mo.). After purification in a Wizard SV Plus kit column (Promega, Madison, Wis.), the DNA was treated with 3 M NaOH and precipitated with three volumes of 100% ethanol, and a one-third volume of 10 M NH₄Oac at room temperature. The precipitated DNA was washed with 70% ethanol and dissolved in 20 μL distilled water. Polymerase chain reaction (PCR) was conducted with primers specific for either the methylated or the unmethylated sequence of IGFBP-3 promoter. Several pairs of primers specific for methylated and unmethylated DNA were selected based on the data from structural analysis of IGFBP-3 promoter showing CpG islands (Cubbage et al., 1990). Methylated primers consisted of IGFBP-3-M (S) 5′-CGAAGTACGGGTTTCGTAGTCG-3′ (SEQ ID NO: 3) and IGFBP-3-M (AS) 5′-CGACCCGAACGCGCCGACC-3′ (SEQ ID NO: 4). Umnethylated primers are comprised of IGFBP-3-U (S) 5′-TTGGTTGTTTAGGGTGAAGTATGGGT-3′ (SEQ ID NO: 5) and IGFBP-3 (AS) 5′-CACCCAACCACAATACTCACATC-3′ (SEQ ID NO: 6). The 25 μL total reaction volume contained 25 ng of modified DNA, 1% dimethyl sulfoxide, all four deoxynucleoside triphosphates (each at 200 μM), 1.5 mM of MgCl₂, 0.4 μM PCR primers, and 0.25 U of HotStar Taq DNA polymerase (Qiagen, Valencia, Calif.). Negative control samples without DNA was included for each set of PCR™. Normal lymphocyte DNA was treated with SssI DNA methyltransferase (New England Biolabs., Inc., Beverly, Mass.), subjected to bisulfite modification, and used as a positive methylated control DNA for each PCR™ reaction. PCR™ of DNA from healthy volunteers served as a negative unmethylated control. For methylated PCR™, DNA was amplified by an initial cycle at 95° C. for 15 min as required for enzyme activation, followed by 40 cycles of 94° C. for 30 seconds, 66° C. for 1 minute, and 72° C. for 1 min and ending with a 5-min extension at 72° C. for 1 min in a themocycler (Applied Biosystems, Foster City, Calif.). For unmethylated PCR™, annealing temperature was substituted with 64° C. PCR™ products were separated on 2.5% agarose gels and visualized after staining with ethidium bromide.

Statistical Analysis

In univariate analysis, independent sample t and chi-square tests were used to analyze continuous and categorical variables, respectively. Survival probability as a function of time was computed by the Kaplan-Meier estimator. The log-rank test was used to compare patient survival time between groups. Overall survival, disease-specific survival (from the date of diagnosis to death specifically from cancer-related causes), and disease-free survival time (from the date of diagnosis to relapse or death of cancer-related causes) were analyzed. Cox regression was used to model the risk of promoter methylation on survival time, with adjustment for clinical and histopathologic parameters (age, sex, tumor histology subgroup, and smoking status). The two-sided test was used to test equal proportion between groups in two-way contingency tables. All statistical tests are two-sided. P<0.05 was considered to be statistically significant.

### Example 6

### Results

Expression of IGFBP-3 and Restoration by 5′-aza-dC Treatment in NSCLC Cells

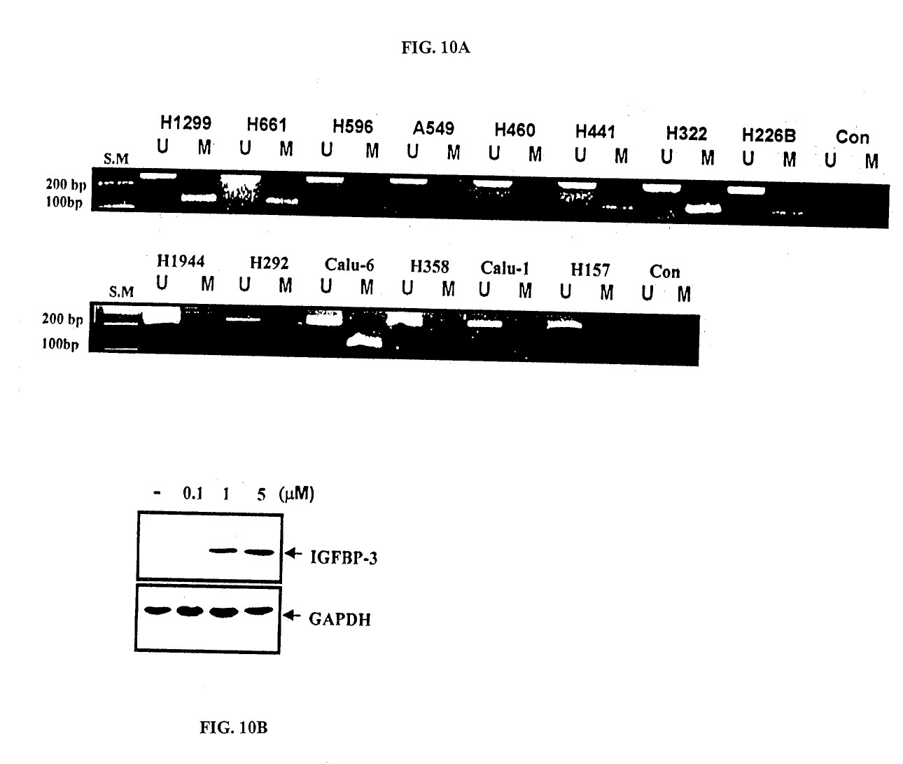

In many cancers, tumor suppressor gene function can be impaired by the loss of gene expression (Tate and Bird, 1993). To determine whether IGFBP-3 expression is down-regulated in NSCLC cells, northern blot analysis on 14 NSCLC cell lines was performed using a probe spanning the entire coding region of IGFBP-3 (FIG. 10A). RNA integrity and equal amounts of RNA in each lane were demonstrated by equivalent intact ribosomal bands in all lanes and the expression of GAPDH. H1299, H661, H441, H322, H292, and Calu-6 cells showed no IGFBP-3 mRNA and H226B, SK-MES-1, and H358 had low expression. The inventor found that the mRNA level of IGFBP-3 in these NSCLC cancer lines reflects the level of IGFBP-3 protein expression. These findings suggest that a major mechanism for the regulation of IGFBP-3 gene expression is at the level of transcription. One potential mechanism that induces the silencing of gene transcription is aberrant methylation of CG dinucleotide near the region of the promoter or the enhancer, a process recently demonstrated for several tumor suppressor genes in lung cancer (Robertson, 2001). Because structural analysis of IGFBP-3 showed CpG islands spanning the region from −250 to 600 bp relative to the mRNA cap site (Cubbage et al. 1990), the inventor investigated whether IGFBP-3 promoter exhibits methylation. First, the expression of IGFBP-3 in H1299 cells, which showed undetectable IGFBP-3 mRNA level, was examined by northern blot analysis after the treatment with the pharmacological agent 5′-aza-dC, which demethylate DNA and activate gene transcription (Magdinier et al., 2000; Traganos et al., 1977). More than 1 μM of 5-aza-dC treatment invariably reactivated IGFBP-3 expression in H1299 cells, supporting the hypothesis that methylation of the CpG island plays an important role in the suppression of IGFBP-3 expression (FIG. 10B).

Methylation-Specific PCR in NSCLC

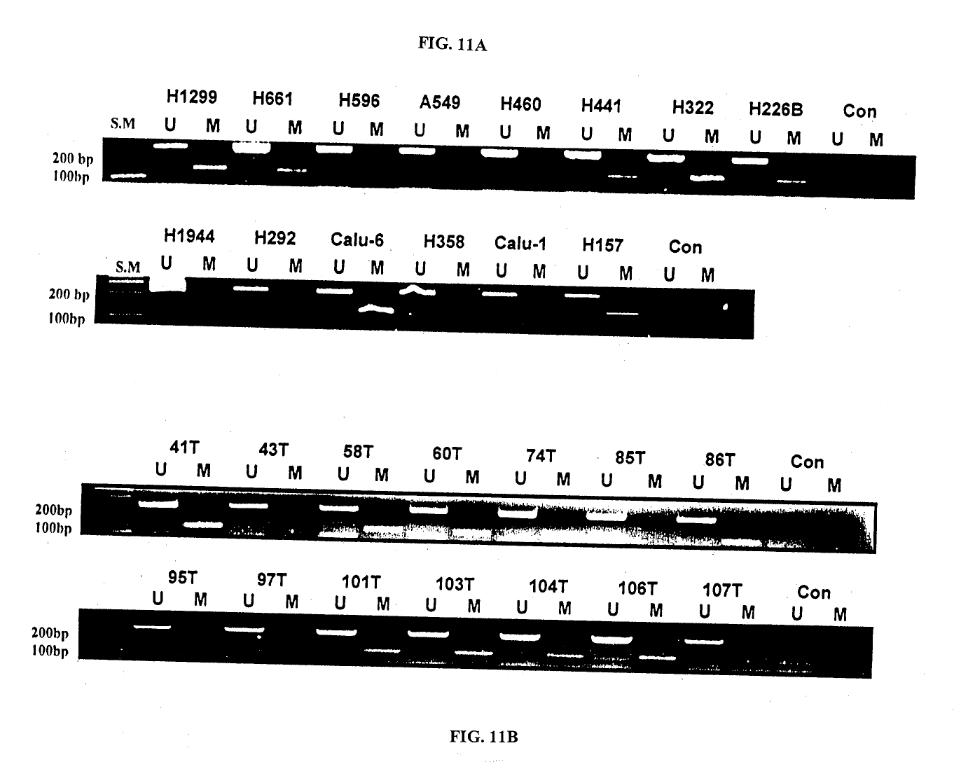

To further investigate whether IGFBP-3 is inactivated by promoter methylation in NSCLC cells, the inventor analyzed CpG islands located at the 5′ flanking site of IGFBP-3 exon1 for methylation by methylation-specific PCR™ (MSP). The DNA from a panel of NSCLC cell lines (H1299, H661, H596, A549, H460, H441, H322, H226B, H1944, H292, Calu-6, H358, Calu-1, and H157) was modified by the treatment of bisulfite and MSP was performed using primers for the methylated and unmethylated forms of IGFBP-3 gene promoter. The primer pair that detects methylated DNA was marked IGFBP-3-M, and the pair that reveals unmethylated DNA was marked IGFBP-3-U. H1299, H661, H441, H322, H226B, and Calu-6 cells, which showed very low or undetectable level of the IGFBP-3 transcript, showed methylation of IGFBP-3 gene promoter (FIG. 11A), suggesting that promoter methylation induces down-regulation of IGFBP-3 expression. Positive signals for methylated DNA (158-bp) as well as for unmethylated DNA (232-bp) were observed in these cell lines, suggesting that these cells are composed of mixed population. In support of this hypothesis, subpopulation of H1299 NSCLC cells that have complete, partial, and unmethylated IGFBP-3 promoter were selected (data not shown). To ensure that successful amplification was not a result of unspecific primer annealing or incomplete bisulfite conversion, PCR™ products from H1299 cells were subjected to direct sequencing, and conversion of all cytosine to thymine at non-CpG sites without change in cytosines at CpG sites was confirmed.

Methylation of IGFBP-3 Promoter in Primary NSCLC and Clinicopathological Characteristics

A total of 123 patients with pathologically confirmed NSCLC were evaluated for the methylation status of the IGFBP-3 promoter. Of the 123 patients, 83 patients had confirmed pathologic stage I NSCLC; 26 patients had stage 1I-IV disease; and in 14 patients, stage was unknown. Using MSP, the inventor analyzed the methylation status of the IGFBP-3 CpG sites located at the 5′-end untranslated region of the gene in the bisulfite-modified genomic DNA from primary tumor samples and 10 bronchial brush samples from volunteers as a control. PCR™ products were separated on a 2.5% agarose gel, and 158-bp and 232-bp PCR™ products were visualized with primers for methylated and unmethylated DNA, respectively. The representative results from 14 tumor samples are shown in FIG. 11B. In tumor samples, either the band that corresponds to methylated IGFBP-3 only or the bands that correspond to both methylated and unmethylated IGFBP-3 were present. Because the tumor sections were microscopically dissected samples that contained more than 70% of tumor cell population as well as nonmalignant tissue, this was expected. PCR™ products obtained from both methylated and unmethylated primer in selected cases were directly analyzed and verified for the expected methylated or unmethylated status by sequencing. The primers for both methylated and unmethylated sequences were tested using unmodified and modified genomic DNA from normal lymphocytes and tumor sections. Unmodified genomic DNA could not be amplified with primers either for methylated or unmethylated DNA. Modified DNA from normal lymphocyte and tumor sections was effectively amplified with primers for unmethylated and methylated DNA, respectively. The inventor found that 72 of 123 NSCLC tissues had methylation in CpG islands at the IGFBP-3 promoter: 51 of 83 at stage I (61.4%), 7 of 9 at stage II (77.8%), 4 of 5 at stage III_{A} (80%); 4 of 6 at stage III_{B} (66.7%), 6 of 6 at stage IV (100%), and 10 of 14 at unknown stage. The methylation of IGFBP-3 was not detected in the DNA from the 10 bronchial brush samples obtained from volunteers. When the inventor analyzed the status of IGFBP-3 methylation in the tumors from NSCLC according to the clinicopathological factors, such as age, histological types, histologic grades, sex, and smoking status, of corresponding patients, there was no statistically significant association among these factors. Smoking status for 96 of 123 patients was available at the time of analysis. All except 4 patients were former smokers, 8 patients stopped smoking before diagnosis but 84 patients still smoked at the time of diagnosis. The mean number of pack-years of the 84 current smokers at the time of diagnosis was 62.9±36.59. There was also no difference in distribution of smoking status or pack-years between the methylated and unmethylated groups.

Methylation of IGFBP-3 and Prognosis

In order to control the other clinical factors including stage, therapeutic interventions, and performance scales that can affect the statistical results, the inventor focused on stage I NSCLC patients in the survival analysis. The age of patients with stage I NSCLC ranged from 39 to 83 years (mean 65.3±8.84 years), which is similar to the age distribution in the large database of NSCLC patients from UTMDACC during the period between 1975 to 1998. Fifty-five (66.3%) of the patients were men and 28 (33.4%) were women, which is also similar to the sex distribution from this and comparable to the gender distribution of the disease in the 1970s and the 1980s (Landis et al., 1998). The probability of 5-year overall survival was 56.7%, which are similar to the probability reported in a previous study with a large number of cases from UTMDACC (Mountain, 1997). Of the 83 patients with stage I NSCLC, 49 patients died, and 34 patients were still alive at the time of the last follow-up report. Of the 49 patients who died, 29 died of lung cancer and 20 died of the other cases. The median follow-up duration was 10.3 years among the patients who remain alive. None of the stage I NSCLC patients received chemotherapy or radiation therapy before or after surgery.

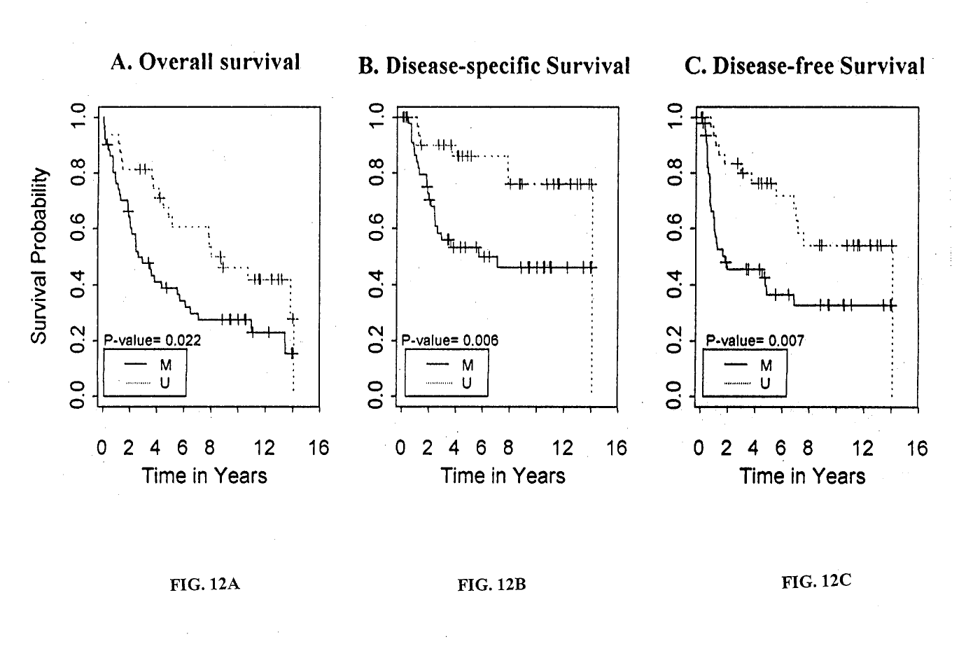

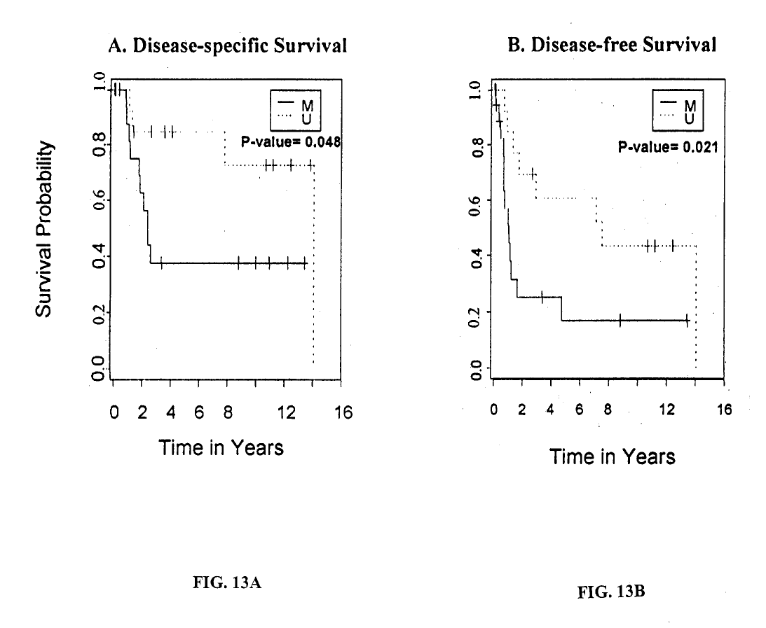

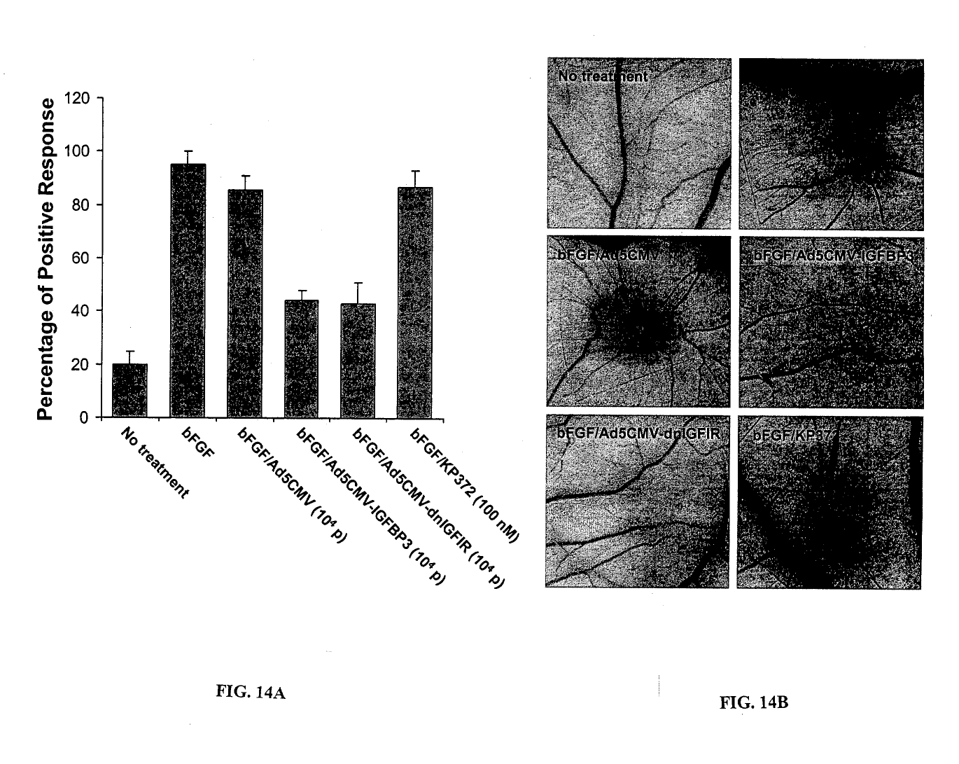

The inventor found that patients whose primary tumors exhibited methylation at the CpG sites of the IGFBP-3 gene had a statistically significant poorer overall survival probability (P=0.022, log-rank test). Furthermore, these patients had poorer disease-specific survival (P=0.006) and disease-free survival probability (P=0.007) compared with the group with the unmethylated gene at 5 years after diagnosis (FIG. 12). The inventor also analyzed potential associations between the methylation pattern and disease-specific and disease-free survival probability in histologic subgroups. The inventor found that methylation was associated with a statistically significant poorer disease-specific and disease-free survival for squamous cell carcinoma (p=0.048 and p=0.021, respectively) (FIG. 13). A similar trend in disease-specific and disease-free survival was observed with other histologic types (i.e., adenocarcinoma, large-cell carcinoma, and unclassified tumors) (P=0.0497 and P=0.055, respectively). To determine whether methylation of IGFBP-3 promoter is an independent factor in predicting survival duration for patients with pathologic stage I NSCLC, the inventor performed multivariate analysis using the Cox model. Promoter methylation status was the only independent predictor for disease-free and disease-specific survival among clinical and histologic parameters tested. Similar trends were shown when all 123 patients with NSCLC were analyzed for disease-specific survival and disease-free survival probability.

### Example 7

### Effect of IGFBP-3 on Angiogenesis and Metastasis

To assess whether IGFBP-3 is able to inhibit angiogenesis chick embryos were induced with IGFBP3 and dnIGFIR. On day 10, each CAM was exposed to bFGF (50 ng/disk) in the absence or presence of 10⁴ particles of Ad5CMV alone, Ad5CMV-IGFBP3, or Ad5CMV-dnIGFIR. After 72 h of incubation, a fat emulsion was injected into the CAM to visualize the blood vessels. CAMs were examined with a microscope to count the positive responsive eggs which have spoke wheel-like vessels. Both IGFBP3 and dnIGFIR inhibited chorioallantoic membrane angiogenesis induced by bFGF FIG. 14. The results are expressed as the percentages of embryos showing activation (FIG. 14A). Representative photographs of disks and surrounding CAMs are shown in FIG. 14B. Positivity for angiogenesis in chick embryos subjected to various conditions was as follows: Collagen alone: 1/5; bFGF (50 ng): 5/5; EV(10⁴)+bFGF: 7/8; IGFBP3(10⁴)+bFGF: 4/9; dnAKT (10⁴)+bFGF: 8/9; dnIGFR (10⁴)+bFGF: 4/9; and KP372 (100 nM)+bFGF: 8/9.

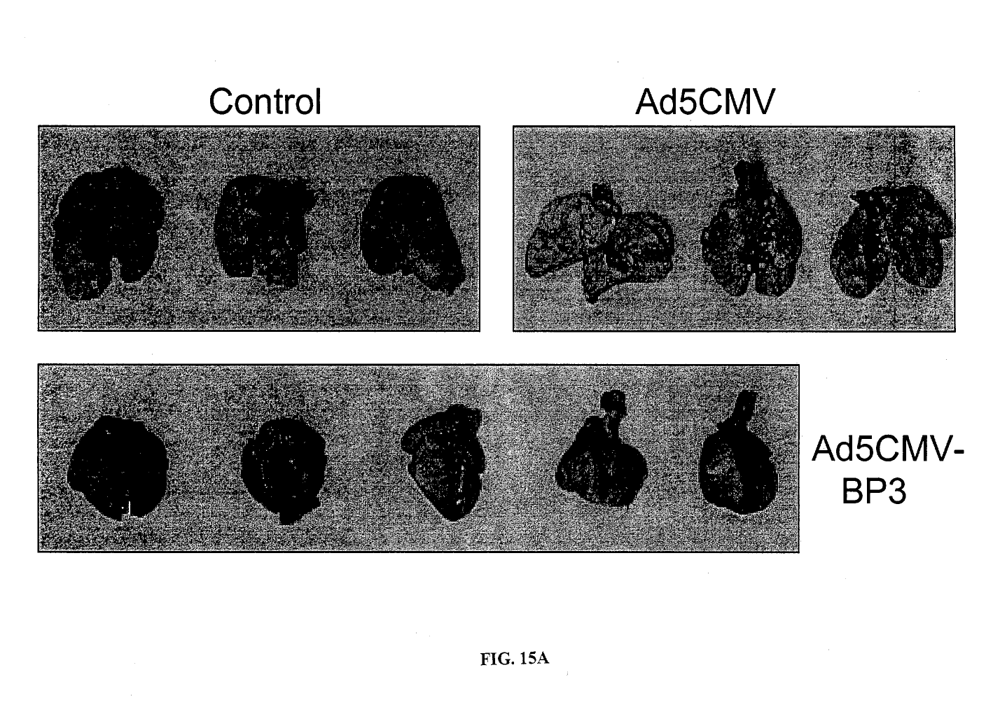

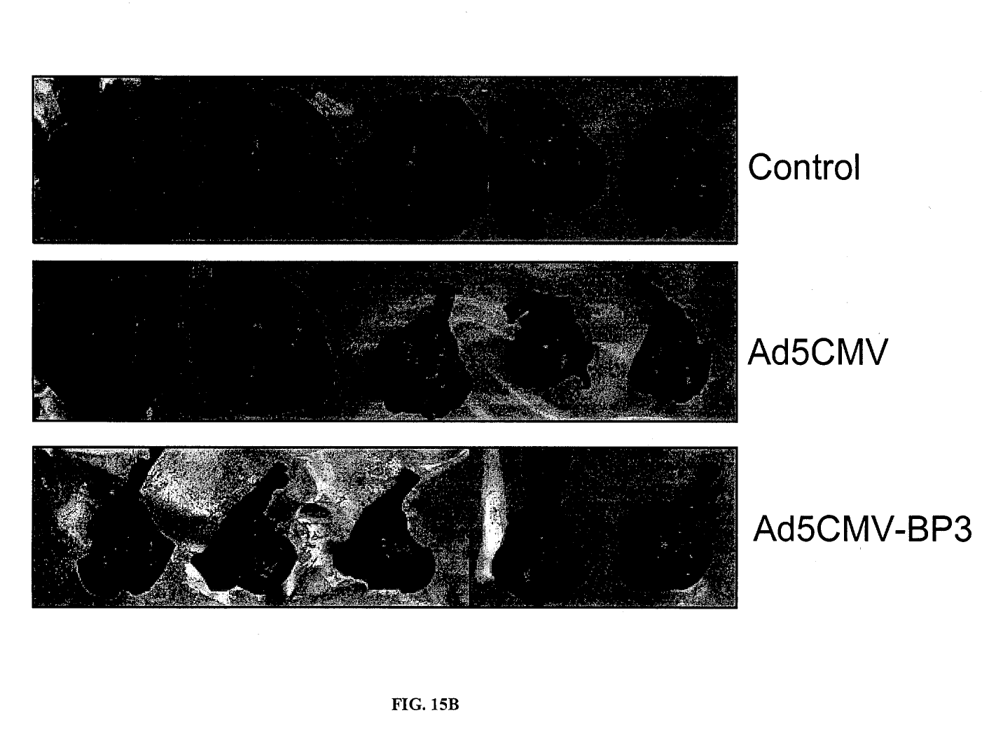

Using a nude mouse model the ability of IGFBP-3 to inhibit metastasis was assessed. A549 (1×10⁶) cells non-infected or infected with empty adenovirus Ad5CMV (10³ particles/cell) or IGFBP3-expressing adenovirus Ad5CMV-IGFBP3 were inoculated i.v. into nude mice. Seven weeks later, the mice were killed, and formation of lung metastases and pleural effusions was evaluated. IGFBP-3 inhibited metastasis in the lung metastasis model of human lung cancer cells in nude mice (FIG. 15A). Similar experiments wer conducted with the rat model which showed that IGFBP-3 inhibited metastasis (FIG. 15B).

### Example 8

### Effect of IGFBP-3 on Head and Neck Squamous Cancer Cell Lines (HNSCC)

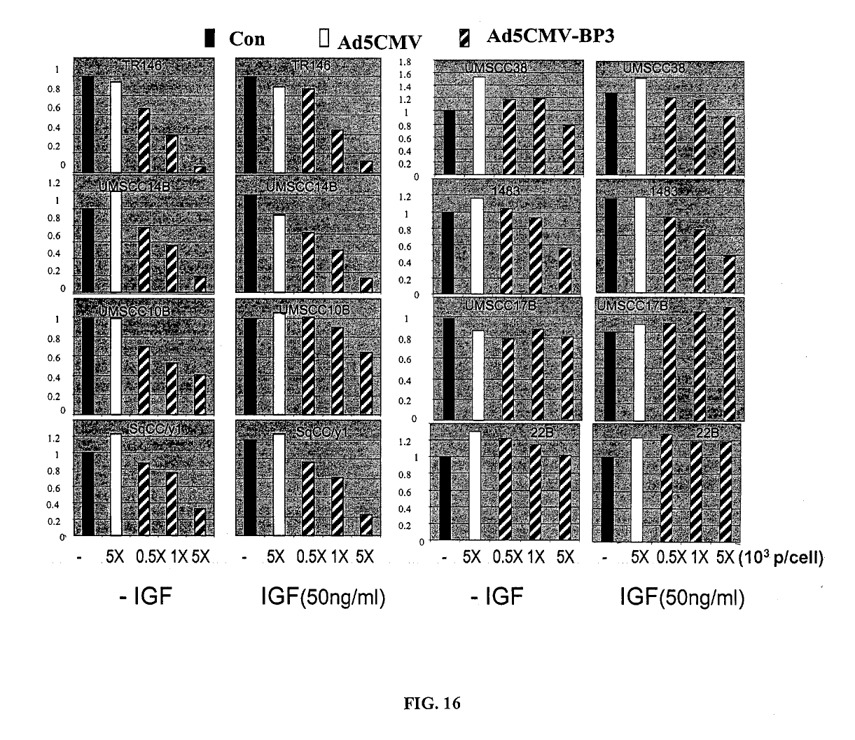

To assess the effect of IGFBP-3 on other cell types, various head and neck cancer cells (TR146, UMSCC38, UMSCC14B, 1483, UMSCC10B, UMSCC17B, SqCC/y1 and 22B) were incubated with varying doses (particles/cell) of Ad5CMV-BP-3 or AdCMV and incubated in serum-free medium with or without 50 ng/ml IGF-1. After 3 days of incubation inhibition of growth was measured using the MTT assay, as described previously, on the infected cells. FIG. 16 shows the results expressed relative to the density of cells incubated in serum-free medium. Each value is the mean (±SD) from six identical wellls.

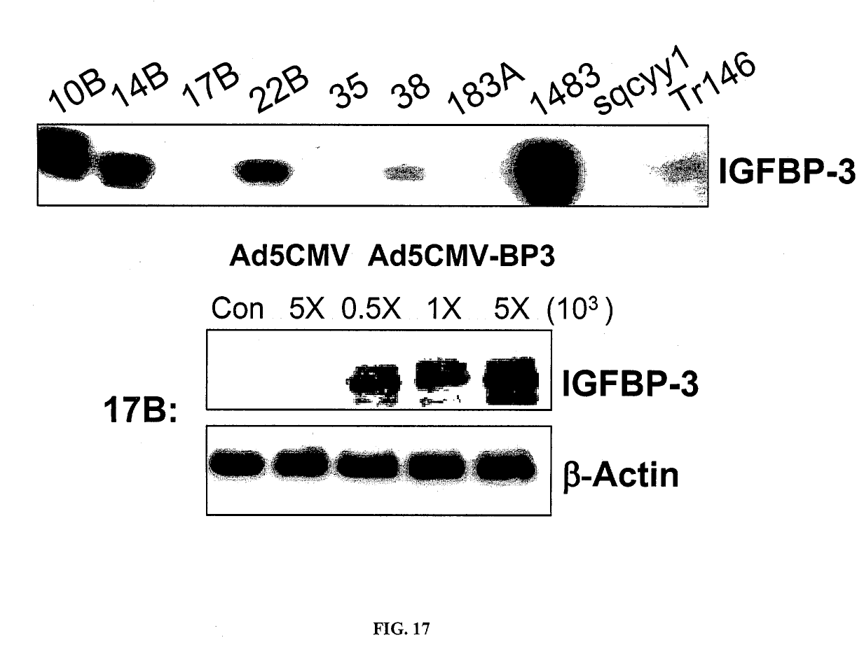

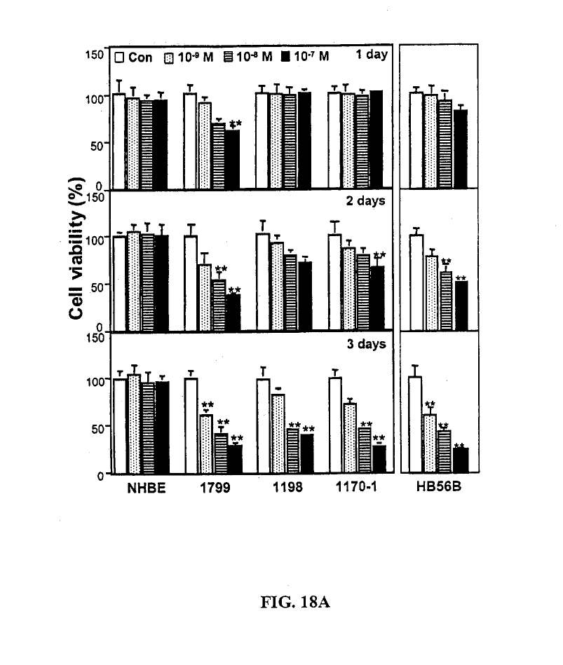

An increase in IGFBP-3 was noted in head and neck cancer cells infected with Ad5CMV-BP-3. Whole cell lysates isolated from indicated head and neck cancer cell lines were electrophoresed, transferred onto nitrocellulose membrane, and incubated with anti-human IGFBP3 antibody. As shown in FIG. 17, a significant increase in expression of IGFBP-3 was noted in the 1483, 10B, 14B, and 22B head and neck cancer cell lines. UMSCC38 and the TR146 cell lines showed slight increase in IGFBP-3 expression.

Further studies were conducted using Ad5CMV-BP3, constructed as previously described using full-length human IGFBP-3 cDNA. The 17B cell line was treated with media alone (con) or infected with the indicated titers (particles/cell) of AdSCMV-BP3 or Ad5CMV for 3 days. Using 10 μg of whole cell lysates, the expression of IGFBP3 in the cell or secreted into medium, was detected by Western blot analysis (FIG. 17).

Additionally, IGFBP-3 was shown to induce apoptosis in HNSCC cells using the 14B cell line. 14B cells infected with varying doses (particles/cell) of AdSCMV (1×10³ or 1×10⁴) or Ad5CMV-BP3 (1×10³ or 1×10⁴) for 3 days, were examined by Western blot analysis, as previously described. Ad5CMV-BP3 was observed to cleaved caspase-3 and PARP in a dose dependent manner, indicating induction of apoptosis by IGFBP-3. AdSCMV empty vector was not able to induce apoptosis in Head and neck cancer cells.

### Example 9

### Modlation of the PI3K Pathway

As discussed in Example 3, IGFBP-3 has the potential to function as an antagonist of the PI3K and MAPK pathways because these pathways are activated by the IGF-1-induced signaling transduction mechanism. The inventor has shown that IGF-I increases the levels of pAkt and pGSK-3β, and induced MAPK activity in H1299 cells. In the present invention, AdSCMV-BP3 has been shown to inhibit IGF-I-induced phosphorylation of Akt (Ser473) and GSK-3β (Ser9) in H1299 cells in a dose-dependent manner. Overall, the results suggested that IGFBP-3 suppressed the IGF-I-induced activation of the PI3K/Akt/PKB and MAPK pathways in H1299 cells. Thus, the inventor investigated targets that may be used as to inhibit the PI3K pathway. As detailed below the inventor provides deguelin as on such compound for the treatment of lung cancer.

### Materials and Methods

Preparation of Deguelin

Deguelin was synthesized in four steps from the natural product rotenone (Sigma-Aldrich, Milwaukee, Wis.), as previously described (Anzenveno, 1979). The final product was more than 98% pure. Deguelin was dissolved in dimethyl sulfoxide (DMSO) at a stock concentration of 10⁻³ M and was stored in a nitrogen tank.

Cells and Cell Cultures

Normal HBE (NHBE) cells were purchased from Clontech (Palo Alto, Calif.) and maintained according to the manufacturer's recommended protocol. BEAS-2B cells, an HBE cell line immortalized with a hybrid adenovirus/simian virus 40 (Reddel et al., 1988) were previously used to derive both premalignant and malignant HBE cells (Klein-Szanto et al., 1992) as follows. BEAS-2B cells were explanted, along with beeswax pellets or beeswax pellets containing cigarette smoke condensate (CSC), into rat tracheas that had been denuded of bronchial epithelium. The tracheas were then transplanted into the dorsal subcutaneous tissues of nude mice (Klein-Szanto et al., 1992). Tumors developed after 6 months. From these tumors, a variety of cell lines were derived in vitro that exhibited different levels of tumorigenicity when transplanted into nude mice. Three cell lines derived from these tumors, the characteristics of which have been described in detail (Klein-Szanto et al., 1992; Kim et al., 1995) were used Two cell lines were premalignant: one (1799) was derived from BEAS-2B cells exposed to a beeswax control pellet, and one (1198) was derived from BEAS-2B cells exposed to a beeswax pellet containing CSC. The third cell line (1170-1) was malignant and was derived from BEAS-2B cells exposed to a beeswax pellet containing CSC.

NHBE cells were induced to differentiate into squamous cells by growing them to confluence on tissue culture plates coated with a thin matrix of fibronectin (10 μg/mL; Upstate Biotechnology, Inc., Lake Placid, N.Y.) and collagen (30 μg/mL; Celtrix Laboratories, Inc., Palo Alto, Calif.) as described (Lee et al., 1996b). HB56B cells were derived from an immortalized HBE cell line induced by loss of a portion of chromosome lip without p53 or K-ras gene mutations (Reddel et al., 1991), NHBE cells, 1799 cells, and squamous HBE cells were grown in keratinocyte serum-free medium (KSFM; Life Technologies Inc., Gaithersburg, Md.) containing EGF (2 μg/mL) and bovine pituitary extract (BPE, 25 μg/mL) (Reddel et al., 1988). 1198 and 1170-1 cells were maintained in KSFM supplemented with 3% fetal bovine serum.

For the analysis of growth inhibition by deguelin, NHBE cells, HBE cell lines, and squamous HBE cells were cultured in KSFM containing EGF and BPE. To induce activation of the phosphatidylinositol 3-kinase (PI3K)/Akt and mitogen-activated protein kinase (MAPK) pathways, H1299 non-small-cell lung cancer (NSCLC) cells, which were purchased from ATCC, and NHBE cells were cultured in the absence of serum and EGF for 1 day and then treated with 50 ng/mL insulin-like growth factor I (IGF-I) for 15 min.

Cell Treatment With Deguelin and Determination of Growth Inhibition

To measure the effects of deguelin on cell proliferation, NHBE, 1799, 1198, 1170-1, and HB56B cells were plated at concentrations of 2×10³ to 4×10³ cells/well in 96-well plates. The next day, cells were treated with either 0.1% DMSO as a diluent control or various concentrations of deguelin (final DMSO concentration=0.1%). At the end of the assay time period, cell proliferation was measured by the 3-(4,5-dimethylthiazol-2-yl)-2,5-diphenyltetrazolium bromide (MTT) assay as described (Lee et al., 2002). Six replicate wells were used for each analysis, and data from replicate wells are presented as means with 95% confidence intervals (CIs). For some experiments, the drug concentration required to inhibit cell growth by 50% (IC₅₀) was determined by interpolation from dose-response curves. At least three independent experiments were performed.

Cell Cycle Analysis

NHBE and 1799 cells were plated at a concentration of 2×10⁵ cells/well in six-well plates. The next day, cells were treated with deguelin (various concentrations) or DMSO (0.1%) for 3 days to achieve maximal antiproliferative effects (determined from growth curves). All cells (nonadherent and adherent) were harvested, fixed with 1% paraformaldehyde and 70% ethanol, stained with 50 μg/mL propidium iodide, and subjected to flow cytometric analysis to determine the percentage of cells in specific phases of the cell cycle (G₁, S, and G₂/M) as described (Sun et al., 1997). Flow cytometric analysis was performed using a Coulter EPICS Profile II flow cytometer (Coulter Corp., Miami, Fla.) equipped with a 488-nm argon laser. Approximately 10 000 events (cells) were evaluated for each sample. Two independent experiments were performed and one is presented.

Apoptosis Assays

NHBE, premalignant (1799 and 1198), and malignant (1170-1) HBE (2×10⁶) cells were exposed to various doses of deguelin or to DMSO (0.1%) for 3 days. Apoptosis was assessed by morphology, by a flow cytometry-based, modified terminal deoxynucleotidyl transferase dUTP nick end labeling (TUNEL) assay, and by the detection of fragmented DNA. For morphology, live cells were observed by light microscopy using a ×100 objective. For TUNEL analysis, both adherent and nonadherent cells were harvested and pooled, fixed with 1% paraformaldehyde and 70% ethanol, and processed using the APO-BrdU staining kit (Phoenix Flow Systems, San Diego, Calif.), a modified TUNEL assay, as described (Lee et al., 2002). Cells treated with DMSO were used to gate the control nonapoptotic populations and as a reference for cells treated with deguelin. An internal control (HL-60 cells treated with camptothecin to induce apoptosis) provided in the apoptosis detection kit was also used to ensure that the TUNEL reaction was occurring during the staining procedure. For the detection of fragmented nucleosomal DNA, cells were processed using the TACS apoptotic DNA laddering kit (Trevigen Inc., Gaithersburg, Md.), according to the manufacturer's recommended protocol.

Immunoblotting

Whole cell lysates from 1×10⁶ cells were prepared in lysis buffer as described (Lonardo et al., 2002). Equivalent amounts of protein were resolved by sodium dodecyl sulfate (SDS)-polyacrylamide gels (7.5%-12%) and transferred to a nitrocellulose membrane. After the membrane was blocked in Tris-buffered saline (TBS) containing 0.05% Tween 20 (TBST) and 5% (w/v) nonfat powdered milk, the membrane was incubated with primary antibody at the appropriate dilution in TBS-5% nonfat milk at 4° C. for 16 h. The membrane was then washed multiple times with TBST and incubated with the appropriate horseradish peroxidaseconjugated secondary antibody for 1 h at room temperature. The protein-antibody complexes were detected by enhanced chemilurminescence (ECL kit; Amersham, Arlington Heights, Ill.), according to the manufacturer's recommended protocol.

The following antibodies and working dilutions were used for the Western blots: rabbit polyclonal antibodies against human phosphorylated Akt (pAkt) (Ser473) (1:1000), Akt (1:1000), phosphorylated glycogen synthase kinase 3β (pGSK-3β) (Ser9) (1:1000), and mouse monoclonal antibody against human antiphosphorylated MAPK (anti-pMAPK) (Thr202/Tyr204) (1:500) (Cell Signaling Technology, Beverly, Mass.); rabbit polyclonal anti-GSK-3α/β (1:1000) (BD Transduction Laboratories, Lexington, Ky.); rabbit polyclonal anti-Bax and anti-caspase-3 antibodies (1:2000) (Pharmingen, San Diego, Calif.); rabbit polyclonal anti-Bcl-2 (1:1000) and rabbit polyclonal antipoly(ADP-ribose) polymerase (PARP) antibody (1:1000) (VIC5; Roche Molecular Biochemicals, Indianapolis, Ind.); rabbit polyclonal anti-hemagglutinin (HA) antibody (1:1000), goat polyclonal antibodies against extracellular related kinase 1(Erk-1; 1:1000), Erk-2 (1:1000), and β-actin (1:4000) (Santa Cruz Biotechnology, Inc., Santa Cruz, Calif.); rabbit anti-mouse immunoglobulin G (IgG)-horseradish peroxidase conjugate (1:2000) (DAKO, Carpinteria, Calif.); and donkey anti-rabbit IgG-horseradish peroxidase conjugate (1:2000) and rabbit anti-goat IgG-horseradish peroxidase conjugate (1:2000) (Amersham Pharmacia Biotech, Arlington Heights, Ill.).

Immune Complex Kinase Assay for MAPK

Approximately 5×10⁶ 1799 cells were treated with 10⁻⁷ M deguelin or 0.1% DMSO for various time periods in the absence of any additional stimulatory growth factors. The cells were then treated with 50 ng/mL IGF-I for 15 minutes to activate MAPK. Total cell extracts were prepared in lysis buffer (Lonardo et al. 2002), and ERK-1/2 or c-Jun N-terminal kinase (JNK) were immunoprecipitated from 100 μg of each cell extract using antibodies (1 μg) that recognize ERK1/2 or JNK (Santa Cruz Biotechnology) and protein-A sepharose beads (20 μL) (Amersham Pharmacia Biotech). Kinase assays were performed by incubating the beads with 30 μL of kinase buffer to which 5 μCi of [γ³²P]ATP (2000 cpm/pmol) and 1 μg of substrate (myelin basic protein [MBP; Calbiochem, La Jolla, Calif.] or GST-cJun [1-79] [Santa Cruz Biotechnology]) were added, as previously described (Lee et al., 2002). The samples were suspended in Laemmli buffer, boiled for 5 min, and then analyzed by SDS-polyacrylamide gel clectrophoresis. The gel was dried and autoradiographed. Total cell lysates from Jurkat cells (Upstate Biotechnology, Inc.) and H1299 NSCLC treated with 50 ng/mL IGF-I for 15 min were used as positive controls. Changes in the level of phosphorylation in MBP or GST-cJun reflect changes in Erk1/2 and JNK activity, respectively.

PI3K Assay

Approximately 5×10⁶ 1799 cells were treated with 10⁻⁷ M deguelin or 0.1% DMSO for different time periods (0-24 h) in the absence of any additional stimulatory growth factors. PI3K in these cells was then activated by treatment with 50 ng/mL IGF-I for 15 min. Cells were lylsed, and PI3K was immunoprecipitated from 500 μg of total cell extracts with 5 μL of rabbit antibody against full-length rat p85 PI3K (Upstate Biotechnology, Inc.), which coprecipitates the p110 catalytic subunit of PI3K, and 20 μL of protein A-sepharose beads (Amersham Pharmacia Biotech). PI3K activity in the immunoprecipitates was analyzed using bovine brain extract (Type I; Sigma-Aldrich), which contains a mixture of phosphatidylinositol, phosphatidylinositol 4-phosphate, and phosphatidylinositol 4,5-bisphosphate as a substrate, as described (Hsu et al., 2000). Jurkat cell lysates (Upstate Biotechnology, Inc.) and IGF-I-activated H11299 NSCLC cell lysates were used as positive controls.

Generation of Adenoviral Vectors and Cell Infection Protocol

Ad5CMV (parental virus) was used as a viral control. An adenoviral vector expressing a full-length Akt (also known as human protein kinase B) with an Src myristoylation signal fused in-frame to the c-Akt coding sequence, and an HA epitope (MyrAkt-HA) (Hsu et al., 2000) under the control of cytomegalovirus (CMV) promoter (Ad5CMV-MyrAkt-HA) was constructed using the pAd-shuttle vector system (Lee et al., 2002). Viral titers were determined by standard plaque assays and spectrophotometric analysis of DNA content. The presence of MyrAkt-HA in viral DNA was confirmed by DNA sequencing of the vector.

Cells were untreated or infected with either Ad5CMVMyrAkt-HA or Ad5CMV as a viral control. Infection was allowed to occur for 2 h in the absence of serum, and then the infected cells were suspended in fresh medium. After 3 days of incubation, the induced expression of MyrAkt-HA in 1799 cells and squamous HBE cells by the adenoviral vector was examined by Western blot analysis for Akt and HA. The function of Ad5CMV-MyrAkt-HA was examined by a Western blot analysis of cell lysates for pGSK-3β (Ser9), which is a downstream target of Akt.

To determine whether deguelin-induced antiproliferative effects on premalignant HBE cells were mediated through the inhibition of the PI3K/Akt pathway, 2×10⁵ 1799 cells/well or 1×10 ⁶ squamous HBE cells/well in six-well plates were infected with 5×10 ³ particles/cell of a control virus (Ad5CMV) or with 1×10³ or 5×10³ particles/cell of Ad5CMV-MyrAkt-HA, an adenoviral vector that expresses a constitutively active Akt. (MyrAkt) in KSFM. After 1 day of infection, cells were treated with 10⁻⁷M or 10⁻⁶M deguelin, 2×10⁻⁶ M or 4×10⁻⁶M N-(4-hydroxyphenyl)retinamide (4-HPR), or 0.1% DMSO as a control and then incubated for 1 or 2 days. Apoptosis was analyzed using the APO-BrdU staining kit for TUNEL, Western blot analyses for caspase-3, and the cleavage of PARP.

Northern Analysis

Approximately 1×10⁷ to 2×10⁷ NHBE cells and squamous HBE cells were lysed in 4 M guanidinium isothiocyanate, and total cellular RNA was extracted as described (Kim et al., 1995). RNA (20 μg per sample) was electrophoresed through a 1% agarose gel containing 2% formaldehyde, transferred to a nylon membrane (Zeta-Probe; Bio-Rad Laboratories, Hercules, Calif.), and hybridized to a [γ³²P] dCTP (2′-deoxycytidine 5′-triphosphate)-labeled transglutaminase (TG) (Polakowska et al., 1991) or involucrine. (Inv) (Eckert et al., 1986) complementary DNA (cDNA), as described (Eckert and Green, 1986). Loading and integrity of each RNA sample was examined by observing the intensity of 18S and 28S in ethidium bromide-stained gels.

Statistical Analysis

Cell survival among groups was compared using Student's t tests. All means and 95% CIs from triplicate samples were calculated using Microsoft Excel software (version 5.0; Microsoft Corporation, Seattle, Wash.). In all statistical analyses, two-sided P values of <0.01 were considered statistically significant.

### Results

Responses of Normal, Premalignant, and Malignant HBE Cells to Deguelin

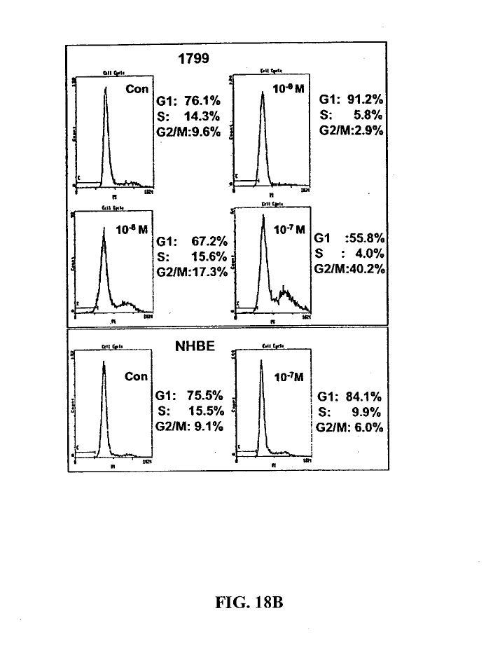

To determine whether deguelin could be a potential lung cancer chemopreventive agent, its effects on the growth of normal, premalignant (1799 and 1198), and malignant (1170-1) HBE cells, which together constitute an in vitro progressive lung carcinogenesis model (Klein-Szanto et al., 1992; Kim et al., 1995) were examined A concentration range of deguelin was used in vitro that was attainable in vivo. The growth of premalignant and malignant HBE cell lines was inhibited by deguelin in a dose- and time-dependent manner (FIG. 18A). After testing a range of concentrations from 10⁻⁹ M to 10⁻⁷M, it was determined that the IC₅₀ for deguelin was less than 10⁻⁸ M. Deguelin had minimal effect on the growth of NHBE cells. Of all the cell lines, premalignant 1799 cells, which represent the earliest stage in the lung cancer model, were the most sensitive to deguelin, with exposure to 10⁻⁷ M deguelin for 1 day decreasing cell growth by 67.1% (95% CI=64.1% to 70.1%). Because BEAS-2B cells have only a few of the properties of premalignant HBE cells in vivo, the effects of deguelin on cells from another immortalized cell line, HB56B were also tested. Dose- and time-dependent growth-inhibitory effects of deguelin in these cells were also detected (FIG. 18A) These results suggest that deguelin preferentially inhibits growth of premalignant HBE cells.

Whether the antiproliferative effects of deguelin were reversible was also examined. 1799 cells were treated with 10⁻⁷ M deguelin for 1, 2, or 3 days and then cultured in medium without deguelin for an additional 5 days. The growth of cells preexposed to deguelin continued to decline during incubation in fresh medium, indicating that the effects of deguelin on cell growth were irreversible. The affect of deguelin on cell growth was assessed by determining the effects of deguelin on the cell cycle using flow cytometry. Cell lines 1799 (FIG. 18B), 1198, and 1170-1 treated with deguelin (10⁻⁸ M or 10⁻⁷ M) for 3 days accumulated in the G₂/M phase of the cell cycle. No detectable cell cycle changes were noted in NHBE cells treated with deguelin (10⁻⁷M) for 3 days.

Effects of Deguelin on Apoptosis In Vitro

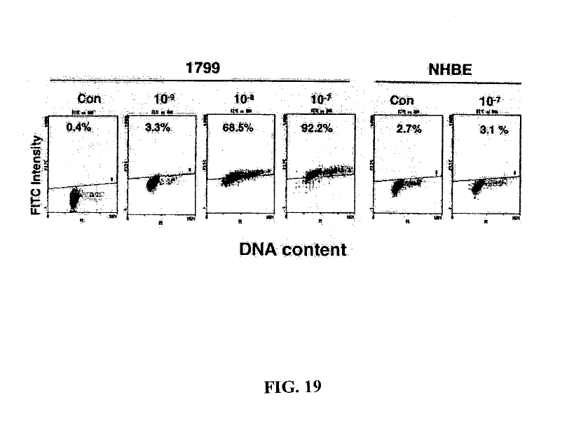

Because cells that accumulate in the G₂/M phase of the cell cycle often enter apoptosis, it was hypothesized that deguelin may have inhibited growth by inducing apoptosis. In fact, cells that were treated with deguelin at greater than 10⁻⁸ M for 1 day showed morphologic changes typical of apoptosis, including membrane blebbing, increased refractoriness, and chromatin condensation. TUNEL staining and flow cytometry analysis confirmed that 1799 cells treated with deguelin were undergoing apoptosis (FIG. 19). Although less than 1% of 1799 cells treated with DMSO underwent apoptosis, approximately 3.3% of 1799 cells treated with 10⁻⁹M deguelin, 68.5% of cells treated with 10⁻⁸ M deguelin, and 92.2% of cells treated with 10⁻⁷ M deguelin for 3 days underwent apoptosis (FIG. 19).

A second test for apoptosis, DNA fragmentation analysis, showed the generation of nucleosomal-sized DNA fragments in 1799 cells treated with deguelin, but not with DMSO. Fragmented DNA was detectable in 1799 cells after treatment with deguelin for 1 day. 1198 and 1170-1 cells treated with deguelin (10⁻⁸ M or 10⁻⁷ M) for 3 days also showed patterns similar to those of 1799 cells in TUNEL and DNA fragmentation analyses; however, treatment with deguelin for 1 day did not induce detectable apoptotic events in these cells. NBDE cells treated with deguelin showed neither a TUNEL-positive cell population nor fragmented DNA.

Next, the inventor assessed the expression of apoptosis-related enzymes (caspase-3 and PARP) and Bcl family members (Bcl-2; Bax, Bcl-xL). There was a decrease in the 32-kd caspase-3 proenzyme and a concomitant increase in the cleavage of the 113-kd fragment of PARP to the 89-kd form in 1799 cells treated with deguelin (10⁻⁸ M or 10⁻⁷ M) for 3 days, indicating that deguelin activated caspase-3. Deguelin also induced a dose dependent increase in the level of Bax and a slight decrease in Bcl-2 expression in 1799 cells but did not affect the level of Bcl-xL. Changes in the levels of these proteins in 1198 and 1170-1 cells treated with deguelin were observed that were similar to the changes observed in 1799 cells (data not shown).

Effect of Deguelin on Components of MAPK and PI3K/Akt Signaling Pathways in Premalignant HBE Cells

The inventor next investigated whether MAPK and PI3K/Akt, which are important in regulating cell apoptosis and proliferation (Robinson and Cobb, 1997; Rodriguez-Viciana et al., 1997; Lee and mcCubrey, 2002,; Nguyen et al., 2000), were involved in deguelin-mediated apoptosis in 1799 cells. Because the activities of MAPK and Akt are regulated by phosphorylation, the levels of phosphorylated MAPK (pP44142 MAPK) and pAkt in NHBE, premalignant (1799 and 1198), and malignant (1170-1) HBE cells treated with deguelin for different time periods were examined. Basal levels of unphosphorylated or phosphorylated MAPK in NHBE, 1799, 1198, and 1170-1 cells were similar. By contrast, basal levels of phosphorylated Akt were higher in premalignant (1799 and 1198) and malignant (1170-1) HBE cells than in NHBE cells, although the levels of the unphosphorylated Akt and an unrelated protein (β-actin) were similar in these cells, indicating that Akt was constitutively active in premalignant and malignant HBE cells. NHBE cells did not express a basal level of phosphorylated Akt; however, IGF-I was shown to induce phosphorylation of Akt in NHBE cells, indicating that the IGF-IR signaling pathway, which leads to Akt phosphorylation, is intact in NHBE cells.

To examine the effects of deguelin on MAPK and PI3K/Akt activities, 1799 cells were treated with 10⁻⁷ M deguelin for different time periods (0-24 h), activated; by treatment with 50 ng/mL IGF-I for 15 min, and then lysed. Erk1/2 was immunoprecipitated with anti-Erk1/2 antibody from the lysates, and kinase activity in the immunoprecipitates was analyzed by using MBP as a substrate. Deguelin had no discernible effect on ERK1/2 activity in 1799 cells. In addition, the activity of JNK, a stress-induced MAPK that plays a role in regulating apoptosis (Davis, 2000; Gajate and Mollinedo, 2002), was not affected by deguelin treatment. Treatment of 1799 cells with 10⁻⁷M deguelin resulted in a time-dependent decrease in the levels of pAkt and pGSK-3β, without affecting the levels of unphosphorylated proteins.

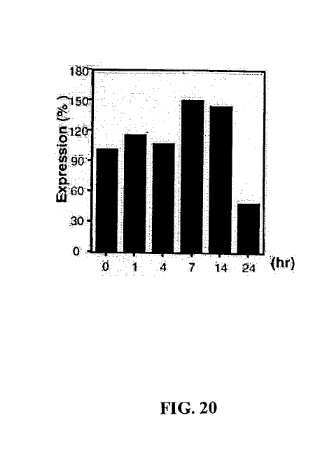

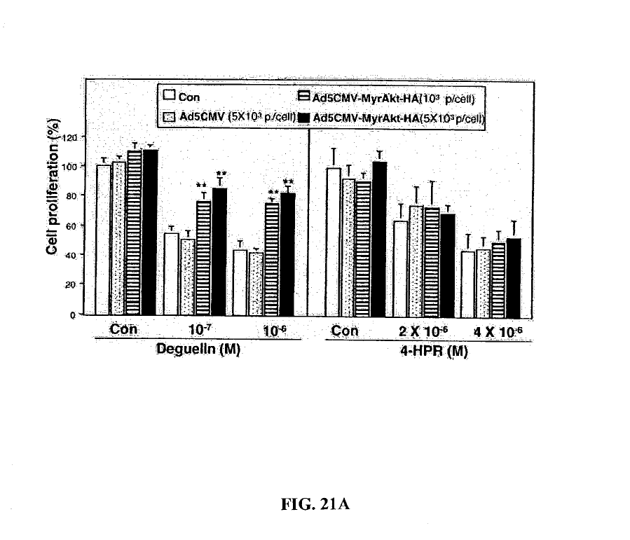

Next the effect of deguelin on PI3K activity was measured. PI3K was immunoprecipitated with anti-p85 antibody from total cell extracts derived from the 1799 cells treated with 10⁻⁷ M deguelin, activated with IGF-11 and tested in a kinase assay. Compared with PI3K activity from untreated cells, deguelin decreased PI3K activity approximately 55%; this decrease was not accompanied by decreased expression of the PI3K components (p85α and p110α) (FIG. 20). Total cell lysates from Jurkat cells and H1299 NSCLC cells treated with 50 ng/mL IGF-I for 15 min and from NHBE cells cultured in the absence of IGF-I activation were used as positive and negative controls, respectively. These findings indicated that deguelin appears to preferentially affect the PI3K/Akt signaling pathway in 1799 cells. Interestingly, the pAkt level was reduced relative to that in untreated cells after 7 h of treatment and was virtually undetectable by 14 h, although PI3K activity was still high during this time period (FIG. 20). These results indicating that deguelin may inhibit Akt activity through PI3K-independent pathways in addition to the PI3K-dependent pathway.

Effect of PI3K/Akt on Deguelin-Induced Death in Premalignant HBE Cells

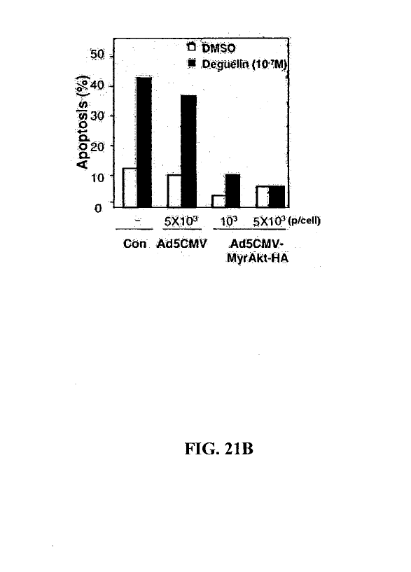

To test the hypothesis that deguelin-induced apoptosis is mediated through the inhibition of the PI3K/Akt pathway, an adenovirus expressing a constitutively active form of Akt (Ad5CMV-MyrAkt-HA) was constructed. Its effects on endogenous Akt expression and activity in 1799 cells was first tested. Dose dependent expression of the HA tag was detected in 1799 cells infected with AdSCMV-MyrAkt-HA. Compared with 1799 cells infected with a control adenovirus (Ad5CMV), expression of MyrAkt-HA, which had a slower mobility (i.e., larger molecular weight) than endogenous Akt, did not affect levels of endogenous Akt. However, the level of pGSK-3β, a downstream Akt target, was increased in 1799 cells infected with Ad5CMV-MyrAkt-HA, thus indicating an increase in Akt activity in these cells. Next, 1799 cells were infected with Ad5CMV-MyrAkt-HA and tested them for susceptibility to treatment with deguelin. 1799 cells infected with Ad5CMVMyrAkt-HA showed an increase in cell survival, relative to cells infected with AdSCMV, in response to deguelin that was dependent on the viral load (FIG. 21A). Compared with the growth of untreated control, growth of 1799 cells, which were uninfected or infected with 5×10³ particles/cell of Ad5CMV and treated with 10⁻⁷ M deguelin for 2 days, was decreased by 54% (95% CI=52.2% to 56.2%). However, growth of 1799 cells infected with 5×10³ particles/cell of Ad5CMV-MyrAkt-HA and treated with 10⁻⁷ M deguelin was 85% (95% CI=81.9% to 88.5%) that of control cell growth. Increasing the concentration of deguelin to 10⁻⁶ M did not decrease the growth of 1799 cells infected with Ad5CMV-MyrAkt-HA.

To test whether PI3K/Akt signaling is important in signal transduction pathways engaged by other pro-apoptotic agents that have effects similar to those of deguelin in HBE cells, the effects of constitutively active Akt on 4-HPRinduced apoptosis was investigated. In 1799 cells, 4-HPR (2-4 μM) concentrations that induce apoptosis did not alter levels of pAkt and pGSK-3β. Furthermore, among cells treated with 4-HPR, there was no difference in cell proliferation between control 1799 cells and 1799 cells overexpressing constitutively active Akt (FIG. 21A). These data indicating that the Akt signaling pathway is not a generic response pathway for chemopreventive agents.

To determine whether expression of Ad5CMV-MyrAkt-HA in 1799 cells affects deguelin-induced apoptosis, apoptosis was assessed in 1799 cells infected with Ad5CMV-MyrAkt-HA and treated with deguelin. Treatment with deguelin (10⁻⁷ M) induced apoptosis in approximately 40% of 1799 cells or 1799 cells infected with the control adenovirus but in less than 10% of 1799 cells infected with Ad5CMV-MyrAkt-HA (FIG. 21B), which indicates that the induction of apoptosis by deguelin in 1799 cells results, at lease in part, from an inhibition of the PI3K/Akt mediated anti-apoptotic pathway.

Effects of Deguelin on Squamous HBE Cells

The premalignant and malignant cell lines used in this study were derived from an HBE cell immortalized with a hybrid adenovirus/simian virus 40 (Klein-Szanto et al., 1992). Adenovirus interaction with αᵥ integrins, an event required for adenovirus internalization, also activates PI3K (Li et al., 1998). Thus, the inventor sought to confirm that the increased level of pAkt in 1799 cells was related to the stage of disease and was not an artifact of the cell line's origin by assessing the level of pAkt in squamous HBE cells. These cells mimic bronchial metaplasia, a potentially premalignant lesion induced by tobacco smoke (Lee et al. 1996).

Squamous HBE cells express higher levels of TG and Inv than NHBE cells (Lee et al., 1996.). To confirm that the cells were indeed induced squamous HBE cells cultured on fibronectin and collagen, expression of TG and Inv in NHBE and squamous HBE cells was assessed by northern blot analysis. It was found that both mRNAs were expressed. Expression of pAkt and pGSK-3β in squamous HBE cells was examined to determine whether the PI3K/Akt pathway was constitutively active in these cells. Although the expression of unphosphorylated Akt and GSK-3α/β was similar in NHBE and squamous HBE cells, the level of pAkt and pGSK-3β was markedly higher in squamous HBE cells than in NHBE cells, indicating that the PI3K/Akt pathway was activated in squamous HBE cells. Whether deguelin would inhibit PI3K/Akt activity in squamous HBE cells was also examined. Treatment with deguelin was observed to decreased the levels of pAkt and pGSK-3β in a time-dependent manner.

The ability of deguelin to induced apoptosis in squamous HBE cells was also examined. Treatment of squamous HBE cells with deguelin (10⁻⁹ M to 10⁻⁷ M) for 1 day induced some of the morphologic changes typical of apoptosis, decrease the inactive form of caspase-3, and concomitantly increased PARP cleavage, all characteristics of cells undergoing apoptosis.

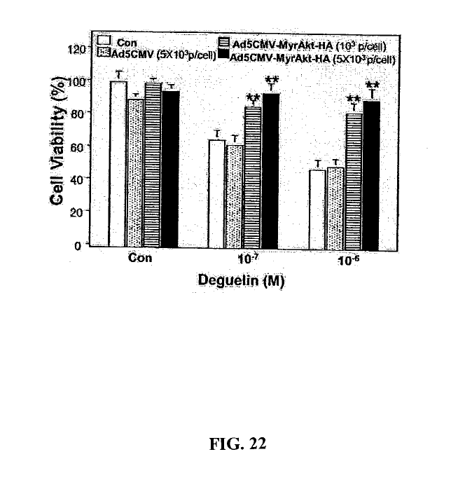

To test whether deguelin induced apoptosis in squamous HBE cells by inhibiting the PI3K/Akt pathway, squamous HBE cells were infected with Ad5CMV or Ad5CMV-MyrAkt-HA and were treated with deguelin (10⁻⁷ M or 10⁻⁶ M) for 1 day. Deguelin decreased proliferation of control squamous HBE cells in a dose-dependent manner (FIG. 22). However, the growth of squamous HBE cells that overexpressed constitutively active Akt and were treated with deguelin (10⁻⁷M to 10⁻⁶M) for 1 day was approximately 95% (95% CI=92.8% to 96.6%) of the growth of untreated cells (FIG. 22). In addition, deguelin induced a loss of caspase-3 and a concomitant increase in PARP cleavage in squamous HBE cells (control or infected with Ad5CMV), but it induced an increase in caspase-3 and a decrease in PARP cleavage in squamous HBE cells infected with Ad5CMV-MyrAkt-HA, indicating that deguelin induction of apoptosis in squamous HBE cells involved inhibition of the PI3K/Akt pathway.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### X. REFERENCES

The following references, to the extent that they provide exemplary procedural or other details supplementary to those set forth herein, are specifically incorporated herein by reference.

U.S. Pat. No. 4,196,265

U.S. Pat. No. 4,554,101

U.S. Pat. No. 4,683,195

U.S. Pat. No. 4,683,202

U.S. Pat. No. 4,800,159

U.S. Pat. No. 4,883,750

U.S. Pat. No. 5,279,721

U.S. Pat. No. 5,354,855

U.S. Pat. No. 5,792,453

U.S. Pat. No. 6,100,242

Agarwal et al., Biol. Reprod., 60:567-572, 1999.

Angel et al., Mol. Cell. Biol., 7:2256, 1987a.

Angel et al., Cell, 49:729, 1987b.

Anzenveno, J. Org. Chem., 44:2578-2580, 1979.

Arap et al., Cancer Res., 55(6):1351-1354, 1995.

Atchison and Perry, Cell, 46:253, 1986.

Atchison and Perry, Cell, 48:121, 1987.

Baichwal and Sugden, In: Gene Transfer, Kucherlapati (ed.), New York, Plenum Press, 117-148, 1986.

Bakhshi, et al., Cell, 41(3):899-906, 1985.

Banerji et al., Cell, 27(2 Pt 1):299-308, 1981.

Banerji et al, Cell, 33(3):729-740, 1983.

Barany and Merrifield, In: The Peptides, Gross and Meienhofer (eds.), Academic Press, New York, 1-284, 1979.

Baserga, Exp. Cell Res., 253:1-6, 1999.

Baylin and Herman, Trends Genet, 16:168-74, 2000.

Bellus, J. Macromol. Sci. Pure Appl. Chem, A31(1): 1355-1376, 1994.

Benvenisty and Neshif, Proc. Natl. Acad. Sci. USA, 83(24):9551-9555, 1986.

Berkhout et al., Cell, 59:273-282, 1989.

Blanar et al., EMBO J., 8:1139, 1989.

Bodine and Ley, EMBO J., 6:2997, 1987.

Boshart et al., Cell, 41:521, 1985.

Bosze et al., EMBO J., 5(7):1615-1623, 1986.

Braddock et al., Cell, 58:269, 1989.

Brodt et al., Biochem. Pharmacol., 60:1101-1107, 2000.

Brognardet al., Cancer Res., 61:3986-3997, 2001.

Brown et al., Oncology Reports, 8(6):1359-62, 2001.

Buckbinder et al., Nature, 377:646-648, 1995.

Bulla and Siddiqui, J. Virol., 62:1437, 1986.

Butt et al., J. Biol. Chem., 275:39174-39181, 2000.

Caldas et al., Nat. Genet., 8(1):27-32, 1994.

Campbell and Villarreal, Mol. Cell. Biol., 8:1993, 1988.

Campbell et al., Am. J. Physiol., 275:E321-E331, 1998.

Campbell, In: Monoclonal Antibody Technology, Laboratory Techniques in Biochemistry and Molecular Biology, Burden and Von Knippenberg (Eds.), Elseview, Amsterdam, 13:71-74; 75-83, 1984.

Campere and Tilghman, Genes and Dev., 3:537, 1989.

Campo et al., Nature, 303:77, 1983.

Capaldi et al., Biochem. Biophys. Res. Comm., 76:425, 1977.

Celander and Haseltine, J. Virology, 61:269, 1987.

Celander et al., J. Virology, 62:1314, 1988.

Chandler et al., Cell, 33:489, 1983.

Chang et al., Hepatology, 14:134A, 1991.

Chang et al., Mol. Cell. Biol., 9:2153, 1989.

Chatterjee et al., Proc Natl. Acad Sci. U.S.A., 86:9114, 1989.

Chen and Okayama, Mol. Cell Biol., 7:2745-2752, 1987.

Chen et al., Oncogene, 17:1959-1968, 1998.

Cheng et al, Cancer Res., 54(21):5547-5551, 1994.

Choi et al., Cell, 53:519, 1988.

Cleary and Sklar, Proc. Natl. Acad. Sci. USA, (21):7439-7443, 1985.

Cleary et al., J. Exp. Med., 164(1):315-320, 1986.

Coffin, In: Virology, Fields et al., eds., Raven Press, NYk, 1437-1500 1990.

Cohen et al., J. Cell. Physiol., 5:75, 1987.

Cohick and Clemmons, J. Cell. Physiol., 161:178-186, 1994.

Conover et al., Endocrinology, 127:2796-2803, 1990.

Costa et al., Mol. Cell. Biol., 8:81, 1988.

Couch et al., Am. Rev. Resp. Dis., 88:394-403, 1963.

Coupar et al., Gene, 68:1-10, 1988.

Cripe et al., EMBO J., 6:3745, 1987.

Crowder and Freeman, J. Neurosci., 18:2933-2943, 1998.

Cubbage et al., J Biol Chem, 265:12642-9, 1990.

Culotta and Hamer, Mol. Cell. Biol., 9:1376, 1989.

D'Ambrosio et al, Cancer Res., 56:4013-4020, 1996.

Dandolo et al., J. Virology, 47:55-64, 1983.

Davis, Cell, 103:239-252, 2000.

De Villiers et al., Nature, 312(5991):242-246, 1984.

Deschamps et al., Science, 230:1174-1177, 1985.

Dubensky et al., Proc. Natl. Acad. Sci. USA, 81:7529-7533, 1984.

Eckert and Green, Cell, 46:583-589, 1986.

Edbrooke et al., Mol. Cell. Biol., 9:1908, 1989.

Edlund et al., Science, 230:912-916, 1985.

Esteller et al., Cancer Res, 58:4515-8, 1998.

European Patent Application EPO 0273085

European Patent Application EPO No. 320 308

Fechheimer et al., Proc. Natl. Acad. Sci. USA, 84:8463-8467, 1987.

Feng and Holland, Nature, 334:6178, 1988.

Ferkol et al., FASEB J., 7:1081-1091, 1993.

Firak and Subramanian, Mol. Cell. Biol, 6:3667, 1986.

Foecking and Hofstetter, Gene, 45(1):101-105, 1986.

Forster and Symons, Cell, 49(2):211-220, 1987.

Fraley et al., Proc Natl. Acad. Sci. USA, 76:3348-3352, 1979.

Freifelder, Physical Biochemistry Applications to Biochemistry and Molecular Biology, 2nd ed. Wm. Freeman and Co., NY, 1982.

Friedman et al., Genes Devel. 3:1314, 1989.

Fujita et al., Cell, 49:357, 1987.

Gajate and Mollinedo, J. Biol. Chem., 277:41580-41589, 2002.

Gefter et al., Somatic Cell Genet, 3(2):231-6, 1977.

Gerber et al., J. Biol. Chem., 273:30336-30343, 1998.

Gerlach et al., Nature (London), 328:802-805, 1987.

Ghosh and Bachhawat, In: Liver Diseases, Targeted Diagnosis and Therapy Using Specific Receptors and Ligands. Wu et al., eds., Marcel Dekker, NY, 87-104, 1991.

Ghosh-Choudhury et al., EMBO J., 6:1733-1739, 1987.

Gilles et al., Cell, 33:717, 1983.

Gloss et al., EMBO J., 6:3735, 1987.

Godbout et al., Mol. Cell. Biol., 8:1169, 1988.

Goding, In: Monoclonal Antibodies: Principles and Practice, 2d ed., Academic Press, Orlando, Fla., pp. 60-61, and 71-74, 1986.

Gomez-Foix et al., J. Biol. Chem., 267:25129-25134, 1992.

Goodboum and Maniatis, Proc. Natl. Acad. Sci. USA, 85:1447, 1988.

Goodboum et al., Cell, 45:601, 1986.

Gopal, Mol. Cell Biol., 5:1188-1190, 1985.

Graham and Prevec, Biotechnology, 20:363-390, 1992.

Graham and Prevec, In: Methods in Molecular Biology: Gene Transfer and Expression Protocol, Murray (Ed.), Humana Press, Clifton, N.J., 7:109-128, 1991.

Graham and van der Eb, Virology, 52:456-467, 1973.

Graham et al., J. Gen. Virol., 36:59-72, 1977.

Greene et al., Immunology Today, 10:272, 1989

Grill and Cohick, J. Cell. Physiol., 183:273-283, 2000.

Grimberg and Cohen, J. Cell. Physiol., 183:1-9, 2000.

Grosschedl and Baltimore, Cell, 41:885, 1985.

Grunhaus et al., Seminar in Virology, 200(2):535-546, 1992.

Hammond, et al., Clin. Res. 42:123A, 1994.

Han et al., J. Biol Chem., 272:13711-13716, 1997.

Harland and Weintraub, J. Cell Biol., 101:1094-1099, 1985.

Harlow and Lane, Antibodies: A Laboratory manual, Cold Spring Harbor Laboratory, 1988.

Haslinger and Karin, Proc. Natl. Acad. Sci. USA, 82:8572, 1985.

Hauber and Cullen, J. Virology, 62:673, 1988.

Hausler et al., Eur. J. Immunol., 28:57-69, 1998.

Hen et al., Nature, 321:249, 1986.

Hensel et al., Lymphokine Res., 8:347, 1989.

Herman et al., Proc Natl Acad Sci USA, 93:9821-9826, 1996.

Hermonat and Muzyczka, Proc. Natl. Acad. Sci. USA, 81:6466-6470, 1984.

Herr and Clarke, Cell, 45:461, 1986.

Hersdorffer et al., DNA Cell Biol., 9:713-723, 1990.

Herz and Gerard, Proc. Natl. Acad. Sci. USA, 90:2812-2816, 1993.

Hirochika et al., J. Virol., 61:2599, 1987.

Hirsch et al., Mol. Cell. Biol., 10:1959, 1990.

Hochscheid et al., J. Endocrinol., 166:553-563, 2000.

Holbrook et al., Virology, 157:211, 1987.

Hollstein et al., Science, 253(5015):49-53, 1991.

Horlick and Benfield, Mol. Cell. Biol., 9:2396, 1989.

Horwich et al. J. Virol., 64:642-650, 1990.

Hsu et al., J. Biol Chem., 275:11397-11403,2000.

Huang et al., Cell, 27:245, 1981.

Hug et al., Mol. Cell. Biol., 8:3065, 1988.

Hussussian et al., Nat. Genet., 8(1):15-21, 1994.

Huynh et al., Int. J. Oncol., 13:137-143, 1998.

Huynh et al., J. Biol. Chem., 271:1016-1021, 1996.

Hwang et al., Mol. Cell. Biol., 10:585, 1990.

Imagawa et al., Cell, 51:251, 1987.

Imbra and Karin, Nature, 323:555, 1986.

Imler et al., Mol. Cell. Biol, 7:2558, 1987.

Imperiale and Nevins, Mol. Cell. Biol, 4:875, 1984.

Innis et al., PCR Protocols, Academic Press, Inc., San Diego Calif., 1990.

Jakobovits et al., Mol. Cell. Biol., 8:2555, 1988.

Jameel and Siddiqui, Mol. Cell. Biol., 6:710, 1986.

Jaques et al., Endocrinology, 138:1767-1770, 1997.

Jaynes et al., Mol. Cell. Biol., 8:62, 1988.

Johnson et al., In: Biotechnology And Pharmacy, Pezzuto et al, eds., Chapman and Hall, New York, 1993.

Johnson et al., Mol. Cell. Biol., 9:3393, 1989.

Jones and Laird, Nat Genet, 21:163-7, 1999.

Jones and Shenk, Cell, 13:181-188, 1978.

Joyce, Nature, 338:217-244, 1989.

Kadesch and Berg, Mol. Cell. Biol., 6:2593, 1986.

Kamb et al., Nat. Genet., 8(1):23-2, 1994.

Kaneda et al., Science, 243:375-378, 1989.

Karin et al., Mol. Cell. Biol., 7:606, 1987.

Karlsson et al., EMBO J, 5:2377-2385, 1986.

Kashiwabara et al., Int. J. Cancer, 79: 215-20, 1998.

Katinka et al., Cell, 20:393, 1980.

Kato et al., J. Biol. Chem., 266:3361-3364, 1991.

Kawamoto et al., Mol. Cell. Biol., 8:267, 1988.

Kennedy et al., Mol. Cell. Biol., 19:5800-5810, 1999.

Kerr et al., Br. J. Cancer, 26(4):239-257, 1972.

Kiledjian et al., Mol. Cell. Biol., 8:145, 1988.

Kim et al., Cancer Res., 55:5603-5610, 1995.

Kim et al., Cancer Res., 57:400-3, 1997.

Kim et al., Cancer Res., 61:3419-24, 2001.

Kim et al., Cancer Res., 55:5603-5610, 1995.

Klamut et al., Mol. Cell. Biol., 10:193, 1990.

Klein et al., Nature, 327:70-73, 1987.

Klein-Szanto et al., Proc. Natl. Acad. Sci. USA, 89:6693-6697, 1992.

Koch et al., Mol. Cell. Biol., 9:303, 1989.

Kohler and Milstein, Eur. J. Immunol., 6:511-519, 1976.

Kohler and Milstein, Nature, 256:495-497, 1975.

Kriegler and Botchan, In: Eukaryotic Viral Vectors, Y. Gluzman, ed., Cold Spring Harbor: Cold Spring Harbor Laboratory, NY, 1982.

Kriegler and Botchan, Mol. Cell. Biol., 3:325, 1983.

Kriegler et al., Cell, 38:483, 1984.

Kriegler et al., Cell, 53:45, 1988.

Kuhl et al., Cell, 50:1057, 1987.

Kulik and Weber, Mol. Cell. Biol., 18:6711-6718, 1998.

Kunz et al., Nucl. Acids Res., 17:1121, 1989.

Kyte and Doolittle, J. Mol Biol, 157(1):105-32, 1982.

Landis, Murray, Bolden, Wingo, Cancer J Clin, 48:6-29, 1998.

Larsen et al., Proc Natl. Acad. Sci. USA., 83:8283, 1986.

Laspia et al., Cell, 59:283, 1989.

Latimer et al., Mol. Cell. Biol., 10:760, 1990.

Le Gal La Salle et al., Science, 259:988-990, 1993.

Leal et al., J. Biol. Chem., 272:20572-20576, 1997.

Lee et al., Cancer Res., 56:3038-3041, 1996.

Lee et al., Cancer Res., 62:3530-3537,2002.

Lee et al., Cell Growth Differ., 7:997-1004, 1996b.

Lee et al., J Clin Invest, 101:10012-1019, 1998.

Lee et al., J. Biol. Chem., 273:7066-7070, 1998.

Lee et al., Leukemia, 16:486-507, 2002.

Lee et al., Nature, 294:228, 1981.

Lee et al., Nucleic Acids Res., 12:4191-206, 1984.

Levinson et al., Nature, 295:79, 1982.

Levrero et al., Gene, 101:195-202, 1991.

Lin et al., Cancer Res., 59:2891-2897, 1999.

Lin et al., Mol. Cell. Biol., 10:850, 1990.

Liu et al., J. Biol. Chem., 275:33607-33613, 2000.

Lonardo et al., Clin. Cancer Res., 8:54-60, 2002.

Luria et al., EMBO J., 6:3307, 1987.

Lusky and Botchan, Proc Natl. Acad. Sci. U.S.A., 83:3609, 1986.

Lusky et al., Mol. Cell. Biol. 3:1108, 1983.

Macaulay et al., Cancer Res., 50:2511-2517, 1990.

Macejak and Sarnow, Nature, 353:90-94, 1991.

Magdinier et al., FASEB J, 14:1585-1594,2000.

Majors and Varmus, Proc. Nat'l Acad. Sci. USA, 80:5866, 1983.

Mann et al., Cell, 33:153-159, 1983.

Mao et al., Nat Med, 2:682-5, 1996.

Markowitz et al., J. Virol., 62:1120-1124, 1988.

McCusker et al., Endocrinology, 129:939-949,1991.

McNeall et al., Gene, 76:81, 1989.

Merlo et al, Nat Med, 1:686-92, 1995.

Merrifield, “Solid phase synthesis,” Science, 232(4748):341-347, 1986.

Michel and Westhof, J. Mol. Biol., 216:585-610, 1990.

Miksicek et al., Cell, 46:203, 1986.

Mitsudomi et al., Oncogene, 7:171-180, 1992.

Mordacq and Linzer, Genes and Dev., 3:760, 1989.

Moreau et al., Nucl. Acids Res., 9:6047, 1981.

Mountain, Chest, 96(1 Suppl): 47S-49S, 1989.

Muesing et al., Cell, 48:691, 1987.

Nemunatis et al., J. Clin. Oncol., 18:609-622, 2000.

Ng et al., Nuc. Acids Res., 17:601, 1989.

Nguyen et al., Oncogene, 19:5385-5397, 2000.

Nicolas and Rubinstein, In: Vectors: A survey of molecular cloning vectors and their uses, Stoneham: Butterworth, pp. 494-513, 1988.

Nicolau and Sene, Biochim. Biophys. Acta, 721:185-190, 1982.

Nicolau et al., Methods Enzymol., 149:157-176, 1987.

Nobori et al., Nature, 368(6473):753-756, 1994.

Nobori et al., Nature, 368(6473):753-756, 1994.

Oh et al., J. Biol. Chem., 270:13589-13592,1995.

Ondek et al., EMBO J., 6:1017, 1987.

Orlow et al., Cancer Res, 54(11):2848-2851, 1994.

Ornitz et al., Mol. Cell. Biol., 7:3466, 1987.

Palmiter et al., Nature, 300:611, 1982.

Paskind et al., Virology, 67:242-248, 1975.

PCT Application WO 84/03564

PCT Application WO 90/07641, 1990

Pech et al., Mol Cell. Biol., 9:396, 1989.

Pelletier and Sonenberg, Nature, 334:320-325, 1988.

Perales et al., Proc. Nat'l Acad. Sci., 91:4086-4090, 1994.

Perez-Stable and Constantini, Mol. Cell Biol., 10:1116, 1990.

Picard and Schaffier, Nature, 307:83, 1984.

Pignon et al., Hum. Mutat., 3: 126-132, 1994.

Pinkert et al., Genes and Dev., 1:268, 1987.

Piyathilake et al., Human Pathology, 32:856-62, 2001.

Polakowska et al., J. Invest. Dermatol., 96:285-288, 1991.

Ponta et al., Proc. Nat'l Acad. Sci. USA, 82:1020, 1985.

Porton et al., Mol. Cell. Biol., 10:1076, 1990.

Potter et al., Proc. Nat'l Acad. Sci. USA, 81:7161-7165, 1984.

Pratt and Pollak, Biochem, Biophys. Res. Commun., 3:292-297, 1994.

Queen and Baltimore, Cell, 35:741, 1983.

Quinn et al., Mol. Cell. Biol., 9:4713, 1989.

Racher et al., Biotechnology Techniques, 9:169-174, 1995.

Ragot et al., Nature, 361:647-650, 1993.

Rajah et al., J. Cell Biol., 272: 12181-12188, 1997.

Reddel et al., Cancer Res., 48:1904-1909, 1988.

Reddel et al., Int. J. Cancer, 48:764-773, 1991.

Redondo et al., Science, 247:1225, 1990.

Reinhold-Hurek and Shub, Nature, 357:173-176, 1992.

Reisman and Rotter, Mol. Cell. Biol, 9:3571, 1989.

Remington's Pharmaceutical Sciences, 15^{th} ed., pages 1035-1038 and 1570-1580, Mack Publishing Company, Easton, Pa., 1980.

Renan, Radiother. Oncol., 19:197-218, 1990.

Resendez Jr. et al., Mol. Cell. Biol., 8:4579,1988.

Rich et al., Hum. Gene Ther., 4:461-476, 1993.

Ridgeway, In: Vectors: A survey of molecular cloning vectors and their uses, Stoneham: Butterworth, pp. 467-492, 1988.

Ripe et al., Mol. Cell. Biol., 9:2224, 1989.

Rippe et al., Mol. Cell Biol., 10:689-695, 1990.

Rittling et al., Nuc. Acids Res., 17:1619, 1989.

Robertson and Wolffe, Nat Rev Genet, 1:11-9, 2000.

Robertson, Oncogene, 20:3139-55,2001.

Robinson and Cobb, Curr. Opin. Cell Biol., 9:180-186, 1997.

Rodriguez-Viciana et al., Cell, 89:457-467, 1997.

Romgnolo et al., Mol. Endocrinol., 102:131-139, 1994.

Rosen et al., Cell, 41:813, 1988.

Rosenfeld et al., Cell, 68:143-155, 1992.

Rosenfeld et al., Science, 252:431-434, 1991.

Roux et al., Proc. Nat'l Acad. Sci. USA, 86:9079-9083, 1989.

Rozen et al., Int. J. Oncol., 13:865-869, 1998.

Rusch et al., Cancer Res., 55:1365-1372, 1995.

Sakai et al., Genes and Dev., 2:1144, 1988.

Sambrook et al., In: Molecular Cloning: A Laboratory Manual, 2nd Ed., Cold Spring Harbor Press, Cold Spring Harbor, N.Y., 1989.

Sarver et al., Science, 247:1222-1225, 1990.

Satake et al., J. Virology, 62:970, 1988.

Scanlon et al., Proc Natl Acad Sci USA, 88:10591-10595, 1991.

Schaffner et al., J. Mol. Biol., 201:81, 1988.

Schedlich et al., J. Biol. Chem., 275:23462-23470, 2000.

Searle et al., Mol. Cell. Biol., 5:1480, 1985.

Serrano et al., Nature, 366:704-707, 1993.

Serrano et al., Science, 267(5195):249-252, 1995.

Sharp and Marciniak, Cell, 59:229, 1989.

Shaul and Ben-Levy, EMBO J., 6:1913, 1987.

Sherman et al., Mol. Cell. Biol, 9:50, 1989.

Sleigh and Lockett, J. EMBO, 4:3831, 1985.

Spalholz et al., Cell, 42:183, 1985.

Spandau and Lee, J. Virology, 62:427, 1988.

Spandidos and Wilkie, EMBO J., 2:1193, 1983.

Stephens and Hentschel, Biochem. J., 248:1, 1987.

Stewart and Young, Solid Phase Peptide Synthesis, 2d. ed., Pierce Chemical Co., 1984.

Stratford-Perricaudet and Perricaudet, In: Human Gene Transfer, Eds, O. Cohen-Haguenauer and M. Boiron, Editions John Libbey Eurotext, France, pp. 51-61, 1991.

Stratford-Perricaudet et al., Hum. Gene. Ther., 1:241-256, 1990.

Stuart et al., Nature, 317:828, 1985.

Sueoka et al., Am. J. Respir. Cell Mol. Biol., 23:297-303, 2000.

Sueoka et al., Oncogene, 19:4432-4436, 2000.

Sullivan and Peterlin, Mol. Cell. Biol, 7:3315, 1987.

Sun et al., J. Cell Physiol., 173:279-284, 1997.

Swartzendruber and Lehman, J. Cell. Physiology, 85:179, 1975.

Takebe et al., Mol. Cell. Biol., 8:466, 1988.

Tam et al., J. Am. Chem. Soc., 105:6442, 1983.

Tang et al., J Natl Cancer Inst, 92:1511-6, 2000.

Tannapfel et al., Oncogene, 20(48):7104-9, 2001.

Tate and Bird, Curr Opin Genet Dev, 1993; 3:226-31, 1993.

Tavernier et al., Nature, 301:634, 1983.

Taylor and Kingston, Mol. Cell. Biol., 10:165, 1990a.

Taylor and Kingston, Mol. Cell. Biol., 10:176, 1990b.

Taylor et al., J. Biol. Chem., 264:15160, 1989.

Temin, In: Gene Transfer, New York: Plenum Press, pp. 149-188, 1986.

Thiesen et al., J. Virology, 62:614, 1988.

Top et al., J. Infect. Dis., 124:155-160, 1971.

Toyota et al., Proc Natl Acad Sci USA, 96:8681-6, 1999.

Traganos et al., Cancer Res,. 41:780-9, 1977.

Treisman, Cell, 42:889, 1985.

Tronche et al., Mol. Biol. Med., 7:173, 1990.

Trudel and Constantini, Genes and Dev. 6:954, 1987.

Tsujimoto and Croce, Proc Natl Acad Sci USA. 83(14):5214-5218, 1986.

Tsukamoto et. al, Science, 229:1390, 1985.

Tur-Kaspa et al., Mol. Cell Biol., 6:716-718, 1986.

Tyndell et al., Nuc. Acids. Res., 9:6231, 1981.

Valentinis et al., Mol. Endocrinol., 9:361-367, 1995.

Vannice and Levinson, J. Virology, 62:1305, 1988.

Varmus et al., Cell, 25:23-36, 1981.

Vasseur et al., Proc Natl. Acad. Sci. U.S.A., 77:1068, 1980.

Wagner et al., Proc. Nat'l Acad. Sci. USA 87(9):3410-3414, 1990.

Walker et al., Endocrinology, 142:3817-3827, 2001.

Wang and Calame, Cell, 47:241, 1986.

Wang et al., Endocrinology, 139: 1354-1360, 1998.

Weber et al., Cell, 36:983, 1984.

Weinberg, Science, 254(5035):1138-1146, 1991.

Weinberger et al. Mol. Cell. Biol., 8:988, 1984.

Winoto and Baltimore, Cell 59:649, 1989.

Wong et al., Gene, 10:87-94, 1980.

Wood et al., Mol. Endocrinol., 2:1176-1185, 1988.

Wu and Wu, Adv. Drug Delivery Rev., 12:159-167, 1993.

Wu and Wu, Biochemistry, 27:887-892, 1988.

Wu and Wu, J. Biol. Chem., 262:4429-4432, 1987.

Wu et al., Genomics, 4:560-569, 1989.

Yang et al., Proc Nat'l Acad Sci. USA, 87:9568-9572, 1990.

Yu et al., J. Natl. Cancer Inst., 91:151-156, 1999.

Yutzey et al. Mol. Cell. Biol., 9:1397, 1989.

Zelenin et al., FEBS Lett., 280:94-96, 1991.

Zeschnigk et al., J. Med. Genet. 36(10):793-4 1999.

Zhang et al., Cancer Gene Ther., 1:5-13, 1994.

Zia et al., J. Cell Biochem. Suppl., 24:269-275, 1996.

Zochbauer-Muller et al., Cancer Res, 61:249-255, 2001.

What is claimed is:

1. A method for inhibiting the growth of a lung cancer cell comprising contacting said cell with an IGFBP-3.

2. The method of claim 1, wherein said lung cancer cell is a non-small cell lung cancer cell.

3. The method of claim 1, further comprising contacting said lung cancer cell with a chemotherapeutic.

4. The method of claim 1, further comprising contacting said lung cancer cell with radiotherapy.

5. The method of claim 1, further comprising contacting said lung cancer cell with a non-IGFBP-3 gene therapy.

6. The method of claim 1, wherein IGFBP-3 is contacted with said lung cancer cell in a pharmaceutical formulation.

7. The method of claim 1, wherein IGFBP-3 is produced from a vector in said lung cancer cell, said vector comprising an IGFBP-3-encoding nucleic acid under the control of a promoter active in said hyperproliferative cell.

8. The method of claim 7, further comprising transferring said vector into said lung cancer cell.

9. The method of claim 7, wherein the vector is a non-viral vector.

10. The method of claim 9, wherein the non-viral vector is encapsulated in a lipid.

11. The method of claim 7, wherein the vector is a viral vector.

12. The method of claim 11, wherein the viral vector is selected from the group consisting of an adenoviral vector, an adeno-associated viral vector, a retroviral vector, a herpesviral vector, a vaccinia viral vector and a papillomavirus vector.

13. The method of claim 12, wherein the viral vector is an adenoviral vector.

14. The method of claim 13, wherein the adenoviral vector is replication-deficient.

15. The method of claim 14, wherein the adenoviral vector lacks at least a portion of the E1 region.

16. The method of claim 15, wherein the nucleic acid encoding IGFBP-3 is inserted in the E1 region.

17. The method of claim 7, wherein said nucleic acid comprises a polyadenylation signal.

18. The method of claim 7, wherein said promoter is an inducible promoter.

19. The method of claim 7, wherein said promoter is a tissue-specific promoter.

20. The method of claim 19, wherein said tissue-specific promoter is a cancer tissue-specific promoter.

21. The method of claim 7, wherein said promoter is a constitutive promoter.

22. The method of claim 21, wherein said constitutive promoter is CMV IE.

23. A method for treating cancer in a subject comprising administering IGFBP-3 to said subject.

24. The method of claim of claim 23, wherein said cancer is lung cancer, breast cancer, pancreatic cancer, liver, cancer, stomach cancer, colon cancer, ovarian cancer, uterine cancer, prostate cancer, testicular cancer, head & neck cancer, skin cancer, brain cancer, esophageal cancer or blood cancer.

25. The method of claim 24, wherein said cancer is lung cancer.

26. The method of claim 25, wherein said lung cancer is non-small cell lung cancer.

27. The method of claim 24, wherein IGFBP-3 is administered to said subject in a pharmaceutical formulation.

28. The method of claim 24, wherein IGFBP-3 is produced from a vector introduced into a cell of said subject, said vector comprising an IGFBP-3-encoding nucleic acid under the control of a promoter active in said hyperproliferative cell.

29. The method of claim 28, wherein the cell of said subject is a cancer cell.

30. The method of claim 28, wherein the vector is a non-viral vector.

31. The method of claim 30, wherein the non-viral vector is encapsulated in a lipid.

32. The method of claim 28, wherein the vector is a viral vector.

33. The method of claim 32, wherein the viral vector is selected from the group consisting of an adenoviral vector, an adeno-associated viral vector, a retroviral vector, a herpesviral vector, a vaccinia viral vector and a papillomavirus vector.

34. The method of claim 27, wherein IGFBP-3 is administered intratumorally.

35. The method of claim 27, wherein IGFBP-3 is administered into tumor vasculature.

36. The method of claim 27, wherein IGFBP-3 is administered regional to a tumor.

37. The method of claim 27, wherein IGFBP-3 is administered systemically.

38. The method of claim 28, wherein IGFBP-3 is administered intratumorally.

39. The method of claim 28, wherein IGFBP-3 is administered into tumor vasculature.

40. The method of claim 28, wherein IGFBP-3 is administered regional to a tumor.

41. The method of claim 28, wherein IGFBP-3 is administered systemically.

42. The method of claim 24, further comprising administering to said subject a second cancer therapy.

43. The method of claim 42, wherein the second cancer therapy is protein therapy, gene therapy, radiation therapy, chemotherapy or surgery.

44. The method of claim 43, wherein the second cancer therapy is protein therapy selected from antibody therapy, cytokine therapy, pro-apoptotic protein therapy, peptide hormone therapy and toxin therapy.

45. The method of claim 43, wherein the second cancer therapy is gene therapy selected from tumor suppressor therapy, antisense oncogene therapy, pro-apoptotic gene therapy, anti-oncogene single-chain antibody gene therapy, cytokine gene therapy, peptide hormone gene therapy and toxin gene therapy.

46. The method of claim 43, wherein the second cancer therapy is radiation therapy selected from gamma irradiation, x-irradiation, ultraviolet irradiation, and microwave irradiation.

47. The method of claim 43, wherein the second cancer therapy is chemotherapy selected from cisplatin, carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl protein tansferase inhibitors, transplatinum, 5-fluorouracil, vincristin, vinblastin and methotrexate.

48. The method of claim 43, wherein the second cancer therapy is surgery.

49. The method of claim 42, further comprising administering to said subject a third cancer therapy.

50. The method of claim 42, wherein the second cancer therapy is provided before IGFBP-3 therapy.

51. The method of claim 42, wherein the second cancer therapy is provided after IGFBP-3 therapy.

52. The method of claim 42, wherein the second cancer therapy is at the same time as IGFBP-3 therapy.

53. A method for predicting or diagnosing cancer in a subject comprising assessing the expression of IGFBP-3 in a cell of said subject, wherein a reduced expression of IGFBP-3, as compared to that seen in a normal cell, is indicate of a risk or presence of cancer.

54. The method of claim 53, wherein said cell is a tumor cell.

55. The method of claim 53, wherein said cell is a non-tumor cell.

56. The method of claim 53, wherein assessing the expression of IGFBP-3 comprises measuring IGFBP-3 protein levels in said cell.

57. The method of claim 53, wherein assessing the expression of IGFBP-3 comprises measuring IGFBP-3 transcript levels in said cell.

58. The method of claim 53, wherein assessing the expression of IGFBP-3 comprises determining the methylation state of the IGFBP-3 promoter.

59. The method of claim 58, wherein determining the methylation state of the IGFBP-3 promoter comprises methylation specific PCR.

60. The method of claim 53, wherein assessing the expression of IGFBP-3 comprises determining the presence of a mutation in the IGFBP-3 coding region.

61. The method of claim 60, wherein determining the presence of a mutation comprises RFLP analysis, sequencing, and DNAse protection.

62. The method of claim 53, further comprising assessing IGFBP-3 expression in a cell from a healthy patient.

63. The method of claim 54, further comprising assessing IGFBP-3 expression in a non-tumor cell from said subject.

64. A method for predicting the efficacy of a cancer therapy on a subject comprising assessing the expression of IGFBP-3 in a cell of said subject.

65. The method of claim 64, wherein assessing the expression of IGFBP-3 comprises determining the methylation state of the IGFBP-3 promoter.

66. The method of claim 65, wherein determining the methylation state of the IGFBP-3 promoter comprises methylation specific PCR.

67. A method for predicting the survival of a subject having cancer comprising assessing the expression of IGFBP-3 in a cell of said subject.

68. The method of claim 67, wherein assessing the expression of IGFBP-3 comprises determining the methylation state of the IGFBP-3 promoter.

69. The method of claim 68, wherein determining the methylation state of the IGFBP-3 promoter comprises methylation specific PCR.

70. A method for predicting the recurrence of a cancer in a subject comprising assessing the expression of IGFBP-3 in a cell of said subject.

71. The method of claim 70, wherein assessing the expression of IGFBP-3 comprises determining the methylation state of the IGFBP-3 promoter.

72. The method of claim 71, wherein determining the methylation state of the IGFBP-3 promoter comprises methylation specific PCR.

73. A method for predicting metastasic cancer in a subject comprising assessing the expression of IGFBP-3 in a cell of said subject.

74. The method of claim 73, wherein assessing the expression of IGFBP-3 comprises determining the methylation state of the IGFBP-3 promoter.

75. The method of claim 74, wherein determining the methylation state of the IGFBP-3 promoter comprises methylation specific PCR.


SEQUENCE LISTING (6 sequences)

SEQ ID NO: 1 - protein, 291 aa, Homo sapiens
MQRARPTLWAAALTLLVLLRGPPVARAGASSGGLGPVVRCEPCDARALAQCAPPPAVCAELVREPGCGCCLTCALSEGQPCGIYTERCGSGLRCQPSPDEARPLQALLDGRGLCVNASAVSRLRAYLLPAPPAPGNASESEEDRSAGSVESPSVSSTHRVSDPKFHPLHSKIIIIKKGHAKDSQRYKVDYESQSTDTQNFSSESKRETEYGPCRREMEDTLNHLKFLNVLSPRGVHIPNCDKKGFYKKKQCRPSKGRKRGFCWCVDKYGQPLPGYTTKGKEDVHCYSMQSK

SEQ ID NO: 2 - DNA, 10884 bp, Artificial Sequence
  Description of Artificial Sequence  Synthetic Primer
ctgcagacctgggacctcaagaattgcatttgatgccgaacccagctctaatttcagagtcaaggtctctgcgagtatttaaggaacggatgtaaacctgggggattcgttttgtttccttcaattttccaatgaaatcagagatcctgttcttgggtgtcaacgcagatactagaaggaggtgatacaagagaaaggaaacagcaagcgacgattatggcacggtttcctgtaaacaaggttgagtgtagccacagcctgagcactgtgggagaagagctcataagaaaatgacggtgctgggccttcgtcaccccggggccctccattgttcttgtctttggtctctttttatttgtagaggtccaattatttatttatttagtacaagagggaacgaaattgatctttccattctaaaaggagagtatatatgtataaaaggaagctgtatagatatgggggaagaggtggacagggggaaaaggggagaggacgagagagagaaagggagggagagggacaaggagagacactgggcgagagatcgattaggagagacagaaatgatgaatgaagattaacttcacccaaggcttcgtcgctggaggggaatggaggagctcctgatttgctattactactccaaactgcaaagggctccttcaagtcacctatccacctcctaaggcaagcgtccaatttcaacagcgttcaggaaagtctcctcccgcggaggtctcaccgcttcccactccacccccacaaactctttggaaaagtgccttgaaaaatttaatcctcaatccaatcctggaccaccagcgtcctctgttggtcaccgaaggagggggtgcgcagacaaaactgaagaaactcgagtgccagagaaggccgacaggagttacagcgacctcagcgcgcaattgcgccccgaactttactgaaaagtgtttagattgcagagataagctagaatcccaacgcatcgagaatacagtaatacgaagtcgccttcaaaaaatgacaatgaaaattgcctattaaaggactatttggttaattacgtttcagcagtgcccagtttattgtctttattattcttttgtcgtgggtgtaaactccatttgaaaacataatcagggagaatacccaagacaagaagaacagttgtcatttaaaatatttgaaaagccctgccttaaggagcattcgcttgccggtccactcttaattggggacttgcggtgtagcaacacgtgagagtcttcttgcgttgagaagtaagcctggaaaggcgaaggccccggggcatcttcagatgcgtatttgtgggcccctggggatataaacagcccagcgggtgtaaattaaaccccgcagtgccttggctccctgagacccaaatgtaagtcagaaatgtcccaagacttcgcctgccaacggaattaaattttagaaagctccacgaggtacacacgaatgcggagcgctgtatgccagtttccccgacaccggctcgccgcagggagacctcaccccgagagcggaaggggtaagggcggcggggtcaaggagatcgggggtgctgagttggccaggagtgactggggtgaccgggggtgctgaggtggcctggagtgccggggtggccgggcacaccttggttcttgtagacgacaaggtgacgggctccgggcgtgagcacgaggagcaggtgcccgggcgagtctcgagctgcacgcccccgagctcggccccggctgctcagggcgaagcacgggccccgcagccgtgcctgcgccgacccgcccccctcccaacccccactcctgggcgcgcgttccggggcgtgtcctgggccaccccggcttctatatacgggccggcgcgcccgggccgcccagatgcgagcactgcggctgggcgctgaggatcagccgcttcctgcctggattccacagcttcgcgccgtgtactgtcgccccatccctgcgcgcccagcctgccaagcagcgtgccccggttgcaggcgtcatgcagcgggcgcgacccacgctctgggccgctgcgctgactctgctggtgctgctccgcgggccgccggtggcgcgggctggcgcgagctcggggggcttgggtcccgtggtgcgctgcgagccgtgcgacgcgcgtgcactggcccagtgcgcgcctccgcccgccgtgtgcgcggagctggtgcgcgagccgggctgcggctgctgcctgacgtgcgcactgagcgagggccagccgtgcggcatctacaccgagcgctgtggctccggccttcgctgccagccgtcgcccgacgaggcgcgaccgctgcaggcgctgctggacggccgcgggctctgcgtcaacgctagtgccgtcagccgcctgcgcgcctacctgctgccagcgccgccagctccaggtgagccgcccgccaggtgcgctgcgtgcagcaccgccactggcgccgaagggcctgggggttgctgggtgccgctgcgggagactccgcttttcttctcactggagataatatgtggggaaactgaaggcgctccgggaaaggtgaaggcggtcgccgagggaccctccccagccggccctctacttgctcgattctctaagtgcagagtacttgtaaattgcaaagcgctttcagtgaaaatgggtaaaggtttccggagctgaggggagcggtaccgatgtttagctgttggaaagatcctggacacaggagattctcctcgccccgcacgggtgcacacggactgcaatcccagggatgcttggggatggggggatataggcggatttggaccaaggaaggtgggtaggcacgttgtaggaaatagtacctctcttttaaaatactgactttgcacagccttttggtttgcaaagcaatgtctagtcccggtatgtccaaaaacaagtaaagtggattcgggttttgatatcttctgcggttggaaaacctgaagctgaaaaagaagtaacttcttaaggttacccagcggccacaacagagtgtaggtttgaactccgcgtgccactttcagtaccataccattcttacaactcgggccacccctgcacctgcgccgacctcaaacaaacttccaggtgcgtggtgggtgcgggcaatgtggactaagtcaatttcaatgacacggcaagggaattggaatcagtcctaggctgtctcccttcttaatctgaaatggggggggggaatgagatgttgttaaggggagccccagaagaggaaaaatgcaaacatttggcagagttaccctcttgcttagccactatcagtatcaggcagacagcgactctggtaagggcatcacattgttcccttaaaaaaaggagcgggggttgtttaaatggatttggcagctgttctttcaagcattcttagccagcctcacctagttatatgagaaataaagttcctgccttgcacagctgaaggctgggagaattctccccatcctaattcccccaactccccaacgatcacgttggacagatgtcactgggcaggcccccatctagggctagcaggatgaacagtccctttataatttatgtagctgtagagttccacgcccgggtgaagttattttctggctcggcaaggctggctctgttcacccctgagaaatgctggattcatggaaaggcaagatgcctgaaacatacactggctctggtcagctgttaaagctgctggaggcatttgtctctcggggcaaagttatgtcatttgccaagtgtcgtacattattgtgcattttggggtattcaaaaagtgatcttagaaatactgatacacatcgtcattcttgggctttagcaatcatcatgattaccaccttagtagcactgtagtataggttgatgtgagttataagattataaaaagatctaagtgacttctagaatctatttgacaaaaaaaggtaaattttcgacagtcaaaagtcacaattatctgttgcttaaatagaactgttttgtcttcatgccctagtctgcagcccaggcattaagaagaaaccaaggaaatttaagaaattactcaaggttcttagaaaagaagtataaatacgtttatttacatgttcttagagtatttacattcttagtatctcttttatctcagtatttccttgaaaaagaaagcaagctaagattaaaagaaattgaaaccaaatcctcgcaggtagggacctcctctgtgaggctctgtgctggaccctgggaatgtgtgcttcccaaggtatgaaaccccttggggaactttacagcaggacctcagtgagctgtttggcaggtgaggaaactaagacccagagaggagagggactttcctaaggccctggtgagtgacctgccagtagccacttccaggggagagcagagcatctgcagccaaatcattgcagccccaggtagctttctagatagactgtggaccagatgggccacctgagctccctgctagggttacacattatagccctgtttgtgtagtagagaaatttcatgactctcaattgtggacttaagccgatgcctccagaccttggcatggtccacaggccctgggagcatgggctctgaatgtagcctttgatccccatagcggtcttacagcccctccaagttcattctgaagaaggaatggagtgagaatcctggctgcagatccagtcttgaatttagtcatatacttaaaattccaattcaactgttaacattccagcatccattttaagcatcagactttcttcatttagcactttttattataaaagggagatctgctggagggggatttctcctaccccacccccacccagggaaggaaaagctctttggcacttagaagtctgagccgtgagtgggactttggcattgtctgcatccatgtgctgctgtgttcacccggggtgaaaaggactcacttaggcaggcaccagcaagatgcacagggtctgtgtagaccttgagttttagagatgtaacggggacctagaaaacaagccaccaacatgcttgcatgattctgagcccctgaggcaaaacgctttgcaggtaataattcagttttcccatctgagctggacaccaagctcttataagcgtgtttacctggtagcattgaggacggtactggtcaaccttggaattcccataagggcttgttacaactcagactcgtgccgccactccagcgtttccggagtggagaatgtgcatttcttccaagtccccgggctgccgctgctcccgcgggtgggaggaccacacttggagttgactgcaaaatttctgagccggcgctgcagcagcctcccgtggctcaggtctgccccctgccggtggaagatgaagcatactgccttcacctactgaggggcactgaagcgtttgtctgccttctttagttgcagctacttaggaagagcacctgtcagattgactttcaaacagataacttcttgaggtagagcaaccaccatgtagtgagtagtatgatggaataatacttcatcgaggtatttaaaaaaaaaacctcacttggattgccaactaatattgtcatttacatgtgacctggttgcaacgttaagatttttacaagactgtgatagatattgatgactctcatgtgtttgtctctcttgggcgttttaaggaaatgctagtgagtcggaggaagaccgcagcgccggcagtgtggagagcccgtccgtctccagcacgcaccgggtgtctgatcccaagttccaccccctccattcaaagataatcatcatcaagaaagggcatgctaaagacagccagcgctacaaagttgactacgagtctcagagcacagatacccagaacttctcctccgagtccaagcgggagacagaatatgtgagagcttttcctcttgttaaaggaggagggcaagacctgccaagcctgggtactcagagcctcttgagggcaattcttactcaacaaaccccagcgcctggctgatgggtgggcaacccctagcccctctgtgccctacctctctcctctccttacataaagaatattgacccttttggagaatcttatgaggatcaagctgaaataacactcttaaaagcatatgggatgtcataaagacctctgcagataatgaaaatattctcataaagatagttttatttacttcatcctctatgcttgttgacctgctattggttccatgccagcttctgtgccttactctgggaagagcaaaaaggagacagggagtgatggttagcttattcgggggactttcgtgctacatcagacataaggtatctgaggagcaaattacaggtcccacttttggtagttgtgcagcatcgtaagatttttaaagcacacattctagagtaaaaactgtgactctgttgctctggtccttcctgatccccagggtccctgccgtagagaaatggaagacacactgaatcacctgaagttcctcaatgtgctgagtcccaggggtgtacacattcccaactgtgacaagaagggattttataagaaaaagcaggtgagtgaggtcctcagtgtgttttcttcctcttctgttgacacagaggagaaacccatgtcaccagcgcccaggctcttgtggccatagctctaactctgagcctgtgcagcaccagtgcccaggacttggtgccagtctcaggaggtcagaccaagggctgctttgacttgttgctctgagtgctgctatattggccataatcctcaaccctagtgcctttccaccacccgcttcccactcctgtcctttcaatggttcacccacaggcggacaagatgctgcccagtggcaccctttataaactgcaagtggacatgttaacacatttgttaatgctgcgtcagggagtgacatttcaaacaactattatagtcagtttccaagaagtgtgacatgaggtcataccacaaaaaagcttaccctgaaatcccacaatcgtcccctttcctactgatgccttcccgatagtgagcaggttgcaatattaagattttgaaaaggctgttgctagatgttggtgactcgtgtgtctctgtctcccttgggcttttcaaggaaatgctagtgagtggggggatgactgcagcatggccagcttggagagcccagccatccccagcacataccaggtgtctgtcttggcgtggaggggatggaacttgaaatcagacactcggtccatgctggggatggccagtctctccaaactggcatgtggtcttcctccgagtcactggcatttccctagaaagtccaagtgagaagaaggcatgagagtcatcaacatcaaacaacagtcttttcaaaatctttatattgcaacatagtcccattcctggaaaaggaatggagtgagaatcctggctacacatcagccccaaatgtagtcattgcctaaaatcccaattaacctgaaaatgatcaaacaaatttaagatatagtaatattaagctgtaataaatatgcttctataggctttgtgttatgtgatggcactatttcaattggctttctaattggacaattgatactatgctatctacagaattggcctttggagacctaagtgagccacagtggcctcagggtgaccatatactaggattcatagcagtggccacagtcagaagcctaagctttcctccattgccattgctcgtttataccacgtttctgtcaaagtcatattcattcaacaaagtcatactgagaaggtgtcatgtgaggctggatgtgggctccaaagtcatagctgtgacattcgcaggcagcgggatgttctcagttccacatttggcagagaagtcagtcaagaggttctacaagggctggtgtccaccttatactcctagaaacacaaaactgcccccacccccgctttcttggagcaggaagttacacccacacgcatgcacaggcgcacactcagcgggcctaggcagcgtggctcttgtgttgccttagctgaaatttctgttgtgctttctcagcatagcagagtcacgctggcaaaccatcatgcgccctggccaccgacctgacaccagacccaggagcattcacttctctgtcttctgtttctctcccacagtgtcgcccttccaaaggcaggaagcggggcttctgctggtgtgtggataagtatgggcagcctctcccaggctacaccaccaaggggaaggaggacgtgcactgctacagcatgcagagcaagtagacgcctgccgcaagggtgagtactcaggaggggcagcctgggctccagggcctcactgtccttggaccagcctcaggggctgggcgtggccactggccttccccaggcttacagacccaggagctgcagctcagggccagaaagagcaaagcaaataggacagagccctcagaagggtgcagggagagggagaccccatcaacccaaccaaacaagtgtggggaaggaggccggccagtgcacctcagggacactctgctttatctcagatacctcacagcacctaagctatcattcatccacacacaaagtgaagattttcaaagttaggctttacccgtgagtctggaggtcatttatcttcacagagaacgtttatcgcagactgctaagatacatgttctaattaagatgtgatgtgagaacgctgaatgctcgttggagactcagttgaagtgcagctttttttctgtcaaatatataatgaatattctgttagtctgtggctaatataattttaataaagttaatttaaatctgatagaaaaatgaaattttaaacgataattttagagaatgctattatatccagtcttcttttttcttttaataaatgagggaactattgggggaaaggaataaatacattttctttcattttattaagacaaatttagtaagcagaagaaatttgcatgtttagttataagggtttcttttttccttacaagttggaaaaaataattctaatttaagggtaactctttgacaatgaacactgtgagcagcatctggtactcgttgctttgtttgaaaacatgagttgagaccccagccgcacttgcagcctagtgccattagcctgcaggctgtgctggatatctcagggcaagagtcgagcccttttgattttggggggattatttcaatatatttgctttttctttttgttttagttaatgtggagctcaaatatgccttattttgcacaaaagactgccaaggacatgaccagcagctggctacagcctcgatttatatttctgtttgtggtgaactgattttttttaaaccaaagtttagaaagaggtttttgaaatgcctatggtttctttgaatggtaaacttgagcatcttttcactttccagtagtcagcaaagagcagtttgaattttcttgtcgcttcctatcaaaatattcagagactcgagcacagcacccagacttcatgcgcccgtggaatgctcaccacatgttggtcgaagcggccgaccactgactttgtgacttaggcggctgtgttgcctatgtagagaacacgcttcacccccactccccgtacagtgcgcacaggctttatcgagaataggaaaacctttaaaccccggtcatccggacatcccaacgcatgctcctggagctcacagccttctgtggtgtcatttctgaaacaagggcgtggatccctcaaccaagaagaatgtttatgtcttcaagtgacctgtactgcttggggactattggagaaaataaggtggagtcctacttgtttaaaaaatatgtatctaagaatgttctagggcactctgggaacctataaaggcaggtatttcgggccctcctcttcaggaatcttcctgaagacatggcccagtcgaaggcccaggatggcttttgctgcggccccgtggggtaggagggacagagagacgggagagtcagcctccacattcagaggcatcacaagtaatggcacaattcttcggatgactgcagaaaatagtgttttgtagttcaacaactcaagacgaagcttatttctgaggataagctctttaaaggcaaagctttattttcatctctcatcttttgtcctccttagcacaatgtaaaaaagaatagtaatatcagaacaggaaggaggaatggcttgctggggagcccatccaggacactgggagcacatagagattcacccatgtttgttgaacttagagtcattctcatgcttttctttataattcacacatatatgcagagaagatatgttcttgttaacattgtatacaacatagccccaaatatagtaagatctatactagataatcctagatgaaatgttagagatgctatatgatacaactgtggccatgactgaggaaaggagctcacgcccagagactgggctgctctcccggaggccaaacccaagaaggtctggcaaagtcaggctcagggagactctgccctgctgcagacctcggtgtggacacacgctgcatagagctctccttgaaaacagaggggtctcaagacattctgcctacctattagcttttctttatttttttaactttttggggggaaaagtatttttgagaagtttgtcttgcaatgtatttataaatagtaaataaagtttttaccattaaaaaaatatctttccctttgttattgaccatctctgggctttgtatcactaattattttattttattatataataattattttattaaaatgttccctgctttcccttttagcaa

SEQ ID NO: 3 - DNA, 22 bp, Artificial Sequence
  Description of Artificial Sequence  Synthetic Primer
cgaagtacgggtttcgtagtcg

SEQ ID NO: 4 - DNA, 19 bp, Artificial Sequence
  Description of Artificial Sequence  Synthetic Primer
cgacccgaacgcgccgacc

SEQ ID NO: 5 - DNA, 26 bp, Artificial Sequence
  Description of Artificial Sequence  Synthetic Primer
ttggttgtttagggtgaagtatgggt

SEQ ID NO: 6 - DNA, 23 bp, Artificial Sequence
  Description of Artificial Sequence  Synthetic Primer
cacccaaccacaatactcacatc